(19) United States
(12) Patent Application Publication
(10) Pub. No.: US 2020/0299671 A1
(43) Pub. Date: Sep. 24, 2020

(54) IMMOBILIZED MULTI-ENZYMATIC HALOGENATION SYSTEM

(71) Applicant: University of Kentucky Research Foundation, Lexington, KY (US)

(72) Inventors: Sylvie Garneau-Tsodikova, Lexington, KY (US); Oleg V. Tsodikov, Lexington, KY (US); Shogo Mori, Lexington, KY (US); Michael D. Burkart, La jolla, CA (US); James J. La Clair, La Jolla, CA (US)

(21) Appl. No.: 16/824,451

(22) Filed: Mar. 19, 2020

Related U.S. Application Data
(60) Provisional application No. 62/820,780, filed on Mar. 19, 2019.

(51) Int. Cl. C12N 11/18; C12N 11/08

(52) U.S. Cl. CPC C12N 11/18; C12N 11/08

(57) ABSTRACT

A halogenation system, a method of halogenating a substrate, and halogenated compounds are provided. The halogenation system includes PltM immobilized on a solid support. The system may include one or more additional enzymes immobilized on the solid support. The method of halogenating a substrate includes running the substrate and a reaction solution through the halogenation system including PltM immobilized on a solid support. The halogenated compounds include 4,6-diCl-3, 4,6-diCl-8, 2,4-diCl-9, 2,6-diCl-11, 3,5-diCl-15, 4,6-diCl-16, 4,6-diCl-18, 4-Cl-23, and/or 4,6-diBr-3.

### RELATED APPLICATIONS

This application claims the benefit of U.S. Provisional Application Ser. No. 62/820,780, filed Mar. 19, 2019, the entire disclosure of which is incorporated herein by this reference.

### GOVERNMENT INTEREST

This invention was made with government support under grant numbers MCB-1149427, awarded by the National Science Foundation (NSF), and UL1TR000117, awarded by the National Institutes of Health (NIH). The Government has certain rights in the invention.

### SEQUENCE LISTING

The instant application contains a Sequence Listing which has been submitted in ASCII format via EFS-Web and is hereby incorporated by reference in its entirety. The ASCII copy of the Sequence Listing, which was created on Mar. 19, 2020, is named 13177N-2357US.txt, and is 14.6 kilobytes in size.

### TECHNICAL FIELD

The present disclosure is directed to a halogenation system. In particular, the disclosure is directed to an immobilized multi-enzymatic halogenation system, methods of use thereof, and modified compounds produced therewith.

### BACKGROUND

Halogenation is an important chemical modification with a potential to increase biological activity and bioavailability of molecules. Moreover, halogen groups can be further synthetically elaborated by transition metal-catalyzed coupling reactions. Halogenase enzymes are attractive potential halogenating tools, because, unlike synthetic halogenation, these enzymes ensure both regiospecificity and green chemistry.

Flavin adenine dinucleotide (FAD)-dependent tryptophan (Trp) halogenases have been the focus of development as halogenation tools. Mutagenesis of Trp halogenase RebH increased its stability, catalytic efficiency, and substrate scope, to halogenate natural products and drug-like molecules. Furthermore, halogenation on a gram scale by this enzyme was achieved by cross-linking it to coupled enzymes. A recent study of the detailed substrate profile of several bacterial Trp halogenases (including RebH) and two fungal phenolic halogenases (Rdc2 and GsfI) indicated that Trp halogenases displayed preference towards indole, phenyl piperidine, phenyl pyrrole, and phenoxyaniline derivatives as substrates, while phenolic halogenases had a narrow substrate profile of some anilines, phenol derivatives, and natural products such as macrolactones and curcumin. While the substrate profiles of some FAD-dependent Trp halogenases appear to be quite broad, the halide spectrum of characterized Trp and phenolic halogenases has been limited to at most two halides: most commonly chloride (Cl—) and bromide (Br—) ions, and for a phenolic halogenase Bmp5, bromide (Br—), and iodide (I—).

In these enzymes, the enzyme-FAD complex catalyzes conversion of a halide ion into a highly reactive hypohalous acid HOX, which diffuses through a protein channel protected from solvent to the substrate binding site, where it is proposed to react with a catalytic lysine residue to form a haloamine adduct, or to form hydrogen bonds with catalytic lysine and glutamic acid residues to act as an active oxidant, with subsequent halogenation of the substrate. FAD is usually a prosthetic group that is tightly and, in some cases, covalently bound to the enzyme, co-purifying with it. Some FAD-dependent halogenases use FAD that can dissociate from the enzyme for reduction (Table 1).

TABLE 1
List of FAD-dependent halogenases with known crystal
structure and their respective substrates

| Main | | Halogenation | |
|---|---|---|---|
| substrate | Halogenase | position | PDB codes* |
| L-tryptophan | PyrH | C5 | 2WES (mutant E46Q, FAD)¹ |
| | | | 2WET (FAD and L-Trp)¹ |
| | | | 2WEU (L-Trp)¹ |
| | MibH | C5 | 5UAO (FAD)² |
| | SttH | C6 | 5HY5 (FAD)³ |
| | PrnA | C7 | 2APG (FAD)⁴ |
| | | | 2AQJ (FAD and L-Trp)⁴ |
| | | | 2AR8 (FAD and 7-Cl-L-Trp)⁴ |
| | | | 2JKC (mutant: E346D, FAD and |
| | | | L-Trp)⁵ |
| | | | 4Z43 (mutant: E450K, FAD)⁶ |
| | | | 4Z44 (mutant: E454K, FAD)⁶ |
| | RebH | C7 | 2O9Z (apo)⁷ |
| | | | 2OA1 (FAD and L-Trp)⁷ |
| | | | 2OAL (FAD)⁸ |
| | | | 2OAM (apo)⁸ |
| | | | 2E4G (L-Trp)⁸ |
| | Th-Hal | C5 and C6 | 5LV9 (apo)⁹ |
| Premalbrancheamide | MalA′ | C9 or C10 | 5WGR (FAD and |
| | | | premalbrancheamide)¹⁰ |
| | | | 5WGS (mutant: H253F, FAD and |
| | | | premalbrancheamide)¹⁰ |
| | | | 5WGT (mutant: H253A, FAD |
| | | | and premalbrancheamide)¹⁰ |
| | | | 5WGU (mutant: E494D, FAD |
| | | | and premalbrancheamide)¹⁰ |
| | | | 5WGV (mutant: C112S/C128S, |
| | | | FAD and premalbrancheamide)¹⁰ |
| | | | 5WGW (FAD and |
| | | | malbrancheamide)¹⁰ |
| | | | 5WGX (mutant: H253A, FAD |
| | | | and malbrancheamide)¹⁰ |
| | | | 5WGY (mutant: C112S/C128S, |
| | | | FAD and malbrancheamide)¹⁰ |
| | | | 5WGZ (FAD and |
| | | | malbrancheamide)¹⁰ |
| Pyrrolyl-S-carrier | PltA | C4 and/or C5 | 5DBJ (FAD)¹¹ |
| protein | Mpy16 | C4 and/or C5 | 5BUK (FAD)¹² |
| | Bmp2 | C3 or C3 and | 5BUL (mutant: |
| | | C4 or C3, | Y302S/F306V/A345W, FAD)¹² |
| | | C4 and C5 | 5BVA (FAD)¹² |
| Tyrosyl-S-carrier | CndH | C3 | 3E1T (FAD)¹³ |
| protein | | | |
| Unknown | CmlS | | 3I3L (FAD)¹⁴ |
| | | | 3NIX (FAD) |

*Bound ligands are FAD, a substrate or a product. Apo refers to the protein not bound to FAD, substrate, or products.

How FAD can dissociate and rebind into the confines of its binding site remains unclear though. Accordingly, there remains a need for an efficient and reusable enzymatic halogenation tool is highly desirable.

### SUMMARY

The presently-disclosed subject matter meets some or all of the above-identified needs, as will become evident to those of ordinary skill in the art after a study of information provided in this document.

This summary describes several embodiments of the presently-disclosed subject matter, and in many cases lists variations and permutations of these embodiments. This summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently-disclosed subject matter, whether listed in this summary or not. To avoid excessive repetition, this summary does not list or suggest all possible combinations of such features.

In some embodiments, the presently-disclosed subject matter includes a halogenation system comprising PltM and a solid support, wherein the PltM is immobilized on the solid support. In some embodiments, the solid support is a resin. In one embodiments, the resin is an agarose resin. In one embodiment, the resin is packed into a spin column. In some embodiments, the halogenation system further includes one or more enzymes immobilized on the solid support. In one embodiment, the one or more enzymes includes a flavin adenine dinucleotide (FAD) reductase. In another embodiment, the FAD reductase includes SsuE. In one embodiment, the one or more enzymes include a NADPH regenerator. In another embodiment, the NADPH regenerator includes glucose dehydrogenase (GDH).

In some embodiments, the halogenation system includes PltM, a flavin adenine dinucleotide (FAD) reductase, a NADPH regenerator, and a solid support, wherein the PltM, the FAD reductase, and the NADPH regenerator are immobilized on the solid support. In some embodiments, the FAD reductase is SsuE. In some embodiments, the NADPH regenerator is glucose dehydrogenase (GDH). In some embodiments, the PltM, SsuE, and GDH are packed into a spin column.

Also provided herein, in some embodiments, is a method of halogenating a substrate, the method comprising running a substrate and reaction solution through the halogenation system including PltM immobilized on a solid support. In some embodiments, halogenation system further comprises SsuE and glucose dehydrogenase (GDH). In some embodiments, the substrate is a phenyl compound with one or more electron donating groups. In one embodiment, the phenyl compound is selected from the group consisting of phenolic derivatives, aniline derivatives, short-acting b2 adrenoreceptor agonists, natural products, and a combination thereof. In some embodiments, the substrate is mono-halogenated. In some embodiments, the substrate is di-halogenated.

Further provided herein, in some embodiments, is a halogenated compound such as 4,6-diCl-3, 4,6-diCl-8, 2,4-diCl-9, 2,6-diCl-11, 3,5-diCl-15, 4,6-diCl-16, 4,6-diCl-18, 4-Cl-23, and/or 4,6-diBr-3.

Further features and advantages of the presently-disclosed subject matter will become evident to those of ordinary skill in the art after a study of the description, figures, and non-limiting examples in this document.

### BRIEF DESCRIPTION OF THE DRAWINGS

The presently-disclosed subject matter will be better understood, and features, aspects and advantages other than those set forth above will become apparent when consideration is given to the following detailed description thereof. Such detailed description makes reference to the following drawings, wherein:

FIG. 1 shows a schematic representation of phloroglucinol (1) halogenation by PltM.

FIGS. 2A-C show graphs illustrating halogenation of phloroglucinol (1) by PltM. (A) XIC traces showing the homo-halogenation of 1 by PltM with NaCl (left), NaBr (middle), and NaI (right) as halide sources. Blue and pink traces depict mono-halogenation and dihalogenation, respectively. (B) Halogenation of 1 by PltM with equimolar ratio of NaCl/NaBr (left), NaCl/NaI (middle), and NaBr/NaI (right). Blue and green traces show mono-halogenation with smaller and larger halogens, respectively; while purple trace indicates dehalogenation. (C) Halogenation of 1 by PltM with NaCl/NaBr (left), NaCl/NaI (middle) and NaBr/NaI (right) in a 10:1 ratio. Blue and green traces show mono-halogenation with smaller and larger halogens, respectively; while purple and red traces indicate homo-dihalogenation with smaller and larger halogens, respectively. The brown trace displays hetero-dihalogenated products. The orange trace shows the unreacted substrate 1.

FIGS. 3A-B show images illustrating substrate profile of PltM by LC-MS. (A) Compounds tested as potential substrates of PltM. (B) Summary of halogenation assay results. The top row and left column indicate the tested substrate and expected halogenation, respectively. Observed and unobserved halogenation are indicated by blue and grey boxes, respectively, while white boxes indicate untested halogenation.

FIGS. 4A-D show images of the crystal structures of PltM. (A) Full view of the structure of PltM with the conserved halogenase fold in pale yellow and the unique C-terminal region in orange. The red loop indicates the N-terminal unconserved region after the 3^{rd} β-sheet. The substrate binding region is shown by a box. (B) A zoomed in view of the substrate binding site of the structure of PltM-compound 1 complex with bound compound 1 (yellow sticks). Residues lining the substrate binding pocket are shown as grey sticks and the mFₒ-DF_{c} polder omit map contoured at 5.5σ is shown by the grey mesh. (C) The FAD bound in the holoenzyme state of PltM. (D) The FAD bound in a putative FAD binding intermediate state. FAD is represented as turquoise sticks in C and D. The flexible loop that changes conformation upon FAD binding is shown in brown. Key FAD interacting residues are shown as sticks. Bound Cl⁻ and water are shown as green and salmon spheres, respectively.

FIGS. 5A-D show graphs illustrating halogenation by PltM and its mutants in a cell-based assay. XIC traces of the cell-based halogenation assay using (A) wild-type PltM, (B) PltM K87A, (C) PltM L111Y, and (D) PltM S404Y. The blue trace refers to the mono-chlorinated 1 while the pink trace shows the dichlorinated 1. The orange trace refers to unmodified starting compound 1.

FIG. 6 shows structures of the products of halogenation by PltM. Structures, as determined by NMR spectroscopy, of products resulting from the halogenation of compounds 3, 8, 9, 11, 15, 16, 18, and 23 by PltM.

FIGS. 7A-B show graphs illustrating HPLC chromatograms of chlorination reactions by PltM with substrates. Reaction with (A) 12, and (B) 23 prior (black traces) and after optimization employing Affi-Gel® resin (pink traces).

FIG. 8 shows LC/MS analysis for the mono- and dihalogenation of compound 1. The top row shows chlorination, the middle row bromination, and the bottom row iodination. The left column shows XIC spectra for 1 (orange), mono-halogenated 1 (blue), and dihalogenated 1 (pink). The middle and right columns show the MS spectra for mono-halogenated 1 and dihalogenated 1, respectively.

FIG. 9 shows LC/MS analysis for the assay 2a (1:1 competition of Cl:Br). The left panel shows the XIC spectrum for 1 (orange), mono-chlorinated 1 (blue), and mono-brominated 1 (green). The middle and right panels show the MS spectra for mono-chlorinated 1 and mono-brominated 1, respectively.

FIG. 10 shows LC/MS analysis for the assay 2b (1:1 competition of CH). The top left panel shows the XIC spectrum for 1 (orange), mono-chlorinated 1 (blue), mono-iodinated 1 (green), and diiodinated 1 (purple). The top middle, top right, and bottom left panels show the MS spectra for mono-chlorinated 1, mono-iodinated 1, and diiodinated 1, respectively.

FIG. 11 shows LC/MS analysis for the assay 2c (1:1 competition of Br:I). The top left panel shows the XIC spectrum for 1 (orange), mono-brominated 1 (blue), mono-iodinated 1 (green), and diiodinated 1 (purple). The top middle, top right, and bottom left panels show the MS spectra for mono-brominated 1, mono-iodinated 1, and diiodinated 1, respectively.

FIG. 12 shows LC/MS analysis for the assay 2d (10:1 competition of CH). The top left panel shows the XIC spectrum for 1 (orange), mono-chlorinated 1 (blue), dichlorinated 1 (pink), mono-iodinated 1 (green), diiodinated 1 (purple), and chloro-iodinated 1 (brown). Inset shows zoom-in of about 35 min. mark to show peak intensity for dichlorinated 1. The top middle, top right, bottom left, bottom middle, and bottom right panels show the MS spectra for mono-chlorinated 1, dichlorinated 1, mono-iodinated 1, diiodinated 1, and chloro-iodinated 1, respectively.

FIG. 13 shows LC/MS analysis for the assay 2e (10:1 competition of Br:I). The top left panel shows the XIC spectrum for 1 (orange), mono-brominated 1 (blue), mono-iodinated 1 (green), and diiodinated 1 (purple). Inset shows zoom-in of about 37.5 min. mark to show peak intensity for diiodinated 1. The top middle, top right, and bottom left panels show the MS spectra for mono-brominated 1, mono-iodinated 1, and diiodinated 1, respectively.

FIG. 14 shows compounds tested as potential substrates of PltM.

FIG. 15 shows MS spectra for compounds 1-15 tested as potential substrates of PltM.

FIG. 16 shows MS spectra for compounds 16-24 tested as potential substrates of PltM.

FIG. 17 shows LC/MS analysis for the mono-chlorination of compound 2. The left column shows XIC spectrum for 2 (orange) and mono-chlorinated 2 (blue). The right column shows the MS spectrum for monochlorinated 2.

FIG. 18 shows LC/MS analysis for the mono- and dihalogenation of compound 3. The top row is for chlorination, and the bottom row is for iodination. The left column shows XIC spectra for 3 (orange), monohalogenated 3 (blue), and dihalogenated 3 (pink). The middle and right columns show the MS spectra for monohalogenated 3 and dihalogenated 3, respectively. Inset shows zoom-in of the peak at 43.131 min for diiodinated 3.

FIG. 19 shows LC/MS analysis for the mono-halogenation of compound 4. The top row is for chlorination, and the bottom row is for iodination. The left column shows XIC spectra for 4 (orange) and mono-halogenated 4 (blue). Inset shows zoom-in of the peak at 34.591 min mono-chlorinated 4. The right column shows the MS spectra for mono-halogenated 4.

FIG. 20 shows LC/MS analysis for the mono-iodination of compound 5. The left column shows XIC spectrum for 5 (orange) and mono-iodinated 5 (blue). Inset shows zoom-in of the peak at 33.935 min for monoiodinated 5. The right column shows the MS spectrum for mono-iodinated 5.

FIG. 21 shows LC/MS analysis for the mono-chlorination of compound 6. The left column shows XIC spectrum for 6 (orange) and mono-chlorinated 6 (blue). The right column shows the MS spectrum for monochlorinated 6.

FIG. 22 shows LC/MS analysis for the mono-halogenation of compound 7. The top row is for chlorination, and the bottom row is for iodination. The left column shows XIC spectra for 7 (orange) and mono-halogenated 7 (blue). The right column shows the MS spectra for mono-halogenated 7.

FIG. 23 shows LC/MS analysis for the mono-halogenation of compound 8. The top row is for chlorination, and the bottom row is for iodination. The left column shows XIC spectra for 8 (orange) and mono-halogenated 8 (blue). The right column shows the MS spectra for mono-halogenated 8.

FIG. 24 shows LC/MS analysis for the mono- and dihalogenation of compound 9. The top row is for chlorination, and the bottom row is for iodination. The left column shows XIC spectra for 9 (orange), monohalogenated 9 (blue), and dihalogenated 9 (pink). Inset shows zoom-in of the peak at 42.605 min for diiodinated 9. The middle and right columns show the MS spectra for mono-halogenated 9 and dihalogenated 9, respectively.

FIG. 25 shows LC/MS analysis for the mono-halogenation of compound 10. The top row is for chlorination, and the bottom row is for iodination. The left column shows XIC spectra for 10 (orange) and mono-halogenated 10 (blue). The right column shows the MS spectra for mono-halogenated 10.

FIG. 26 shows LC/MS analysis for the mono- and dihalogenation of compound 11. The top row is for chlorination, the middle row is for bromination, and the bottom row is for iodination. The left column shows XIC spectra for 11 (orange), mono-halogenated 11 (blue), and dihalogenated 11 (pink). The middle and right columns show the MS spectra for mono-halogenated 11 and dihalogenated 11, respectively.

FIG. 27 shows LC/MS analysis for the mono- and dihalogenation of compound 12. The top row is for chlorination, and the bottom row is for iodination. The left column shows XIC spectra for 12 (orange), monohalogenated 12 (blue), and dihalogenated 12 (pink). The middle and right columns show the MS spectra for monohalogenated 12 and dihalogenated 12, respectively. Inset shows zoom-in of the peak 37.796 min for dichlorinated 12.

FIG. 28 shows LC/MS analysis for the mono-halogenation of compound 13. The top row is for chlorination, and the bottom row is for iodination. The left column shows XIC spectra for 13 (orange) and mono-halogenated 13 (blue). The right column shows the MS spectra for mono-halogenated 13. Inset shows zoom-in of the peak at about 36.198, 36.740, and 36.980 min for mono-iodinated 13.

FIG. 29 shows LC/MS analysis for the mono-iodination of compound 14. The left column shows XIC spectrum for 14 (orange) and mono-iodinated 14 (blue). The right column shows the MS spectrum for monohalogenated 14. Inset shows zoom-in of the peak 37.380 min for mono-iodinated 14.

FIG. 30 shows LC/MS analysis for the mono-halogenation of compound 15. The top row is for chlorination, and the bottom row is for iodination. The left column shows XIC spectra for 15 (orange) and mono-halogenated 15 (blue). The right column shows the MS spectra for mono-halogenated 15. Insets show zoom-in to show peak intensities for chlorinated and iodinated 15 on top and bottom panel, respectively.

FIG. 31 shows LC/MS analysis for the mono- and dihalogenation of compound 16. The top row is for chlorination, and the bottom row is for iodination. The left column shows XIC spectra for 16 (orange), monohalogenated 16 (blue), and dihalogenated 16 (pink). The middle and right columns show the MS spectra for monohalogenated 16 and dihalogenated 16, respectively.

FIG. 32 shows LC/MS analysis for the mono-halogenation of compound 17. The top row is for chlorination, and the bottom row is for iodination. The left column shows XIC spectra for 17 (orange) and mono-halogenated 17 (blue). The right column shows the MS spectra for mono-halogenated 17.

FIG. 33 shows LC/MS analysis for the mono-chlorination of compound 18. The left column shows XIC spectrum for 18 (orange) and mono-chlorinated 18 (blue). The right column shows the MS spectrum for monochlorinated 18. Inset shows zoom-in of the peak at 30.101 min for unmodified 18.

FIG. 34 shows LC/MS analysis for the mono- and diiodination of compound 21. The left column shows XIC spectrum for 21 (orange), mono-iodinated 21 (blue), and diiodinated 21 (pink). The middle and right columns show the MS spectra for mono-iodinated 21. Inset shows zoom-in of the peak at 29.804 min for diiodinated 21.

FIG. 35 shows LC/MS analysis for the mono-iodination of compound 22. The left column shows XIC spectrum for 22 (orange) and mono-iodinated 22 (blue). The right column shows the MS spectrum for mono-iodinated 22. Inset shows zoom-in of the peak at 32.154 min for mono-iodinated 22.

FIG. 36 shows LC/MS analysis for the mono-halogenation of compound 23. The top row is for chlorination, and the bottom row is for iodination. The left column shows XIC spectra for 23 (orange) and mono-halogenated 23 (blue). The right column shows the MS spectra for mono-halogenated 23.

FIG. 37 shows LC/MS analysis for the mono-halogenation of compound 24. The top row is for chlorination, and the bottom row is for iodination. The left column shows XIC spectra for 24 (orange) and mono-halogenated 24 (blue). The right column shows the MS spectra for mono-halogenated 24. Inset shows zoom-in of the peak at 33.137 min for mono-chlorinated 24.

FIG. 38 shows structure-based sequence alignment of PltM, PltA and RebH. The alignment was obtained from the superimposition of crystal structures of PltM (PDB: 6BZN) (SEQ ID NO: 11), PltA (PDB: 5DBJ) (SEQ ID NO: 12), and RebH (PDB: 2OA1) (SEQ ID NO: 13). Conserved residues are shown by red boxes. The conserved FAD binding motifs are underlined and labeled in orange. Residues mutated in this study are indicated by navy rectangles. The catalytic lysine residue, K87 is indicated by a yellow oval. The flexible FAD interacting loop of PltM is highlighted and labeled in brown.

FIGS. 39A-B show PltM preparation and crystals. (A) The S-200 size-exclusion chromatograms of PltM and PltA (left panel). A picture of a 15% SDS-PAGE gel showing purified PltM (right panel). (B) Concentrated PltM and crystals of PltM. Concentrated PltA and its crystals, obtained as described previously, are shown for comparison.

FIGS. 40A-F show structural comparison of the substrate binding site of various FAD-dependent halogenases. (A) The structure of PltM. The conserved N-terminal region is colored pale yellow and the C-terminal region is colored orange. (B) A zoomed in view of the substrate binding site of PltM in complex with compound 1 (yellow sticks). (C) The structure of RebH in complex with bound L-Trp (grey sticks; PDB: 2OA1). The N-terminal and C-terminal regions are in purple and yellow, respectively. (D) A zoomed in view of the substrate binding site of RebH. (E) The structure of PltA (PDB ID: 5DBJ). The N-terminal region is shown in teal, and the C-terminal region occluding the substrate binding site is shown in blue. (F) A zoomed in view of the substrate binding site of PltA.

FIG. 41 shows the in vitro analysis of PltM K87A by using 11 as the substrate. Wild-type PltM yields diCl-11 at these conditions.

FIGS. 42A-B show substrate binding with diionated compound 1. (A) The PltM substrate binding site with a modeled diiodinated compound 1. (B) An alternative view of the substrate binding site with the model of diiodinated compound 1.

FIGS. 43A-F show structural comparison of the FAD binding site of various FAD-dependent halogenases. The FAD binding site is represented as surface and cartoon for (A) and (B) PltM (PDB ID: 6BZQ and 6BZT), (C) and (D) RebH (PDB: 2OA17), (E) and (F) PltA (PDB: 5DBJ11), respectively. The FAD (light blue sticks) is encased by the residues (shown as grey sticks) for PltA. Corresponding residues for PltM and RebH are indicated by grey sticks. The chloride ion is shown as a green sphere.

FIGS. 44A-D show The omit electron density maps for FAD and chloride. The FAD in the structure of PltM and chloride are well defined by the Fₒ-F_{c} omit map contoured at 3σ (maroon mesh) for (A) PltM L111Y and 2.5σ for (B) PltM-WT in complex with fully bound FAD. (C) The isoalloxazine ring is well defined by the Fₒ-F_{c} omit map contoured at 3σ in the complex of PltM with partially bound FAD. (D) A view similar to C, but tilted to show the isoalloxazine ring. This view shows that the right-hand side ring, including the asymmetrically positioned oxygen atoms, is well resolved by the Fₒ-F_{c} omit map, unambiguously defining the orientation of the isoalloxazine ring. This position of the isoalloxazine ring is consistent with the interactions of the nonpolar left-hand side of the ring with surrounding nonpolar residues (Val42, Phe199, Trp239, and Pro328) and the polar right-hand side of the ring with the nearby hydroxyl of Tyr306 and surrounding solvent, as illustrated in panel D.

FIG. 45 shows steric overlap of Tyr at positions 111 and 404 with the substrate binding site. The substrate binding site of PltM L111Y, as observed in the crystal structure of PltM L111Y with the model of bound substrate 1 (as observed in the structure of PltM-substrate 1 complex) and a modeled S404Y mutation. Either tyrosine residue at positions 111 and 404 clashes sterically with substrate 1.

FIG. 46 shows LC/MS analysis of halogenation reaction of 1 in the cell-based assays. The top row shows halogenation with PltM WT, the second row is with PltM L111Y mutant, the third row is with PltM S404Y mutant, and the bottom row is PltM K87A (left) and using PltA (right), which served as the negative control for the experiment. For the top three rows, the left column shows the XIC spectra for 1 (orange), mono-chlorinated 1 (blue), and dichlorinated 1 (pink). The middle and right columns show the MS spectra for mono-chlorinated 1 and dichlorinated 1, respectively.

FIGS. 47A-I show time course experiments by using the new optimized in vitro reaction conditions. (A) Chlorination of substrate 3. (B) Bromination of substrate 3. (C) Iodination of substrate 3. (D) Chlorination of substrate 11. (E) Bromination of substrate 11. (F) Iodination of substrate 11. (G) Chlorination of substrate 16. (H) Bromination of substrate 16. (I) Iodination of substrate 16. The orange circles, blue triangles, green squares, and pink diamonds indicate the % distribution of substrate, one mono-halogenated substrate, the second mono-halogenated substrate, and the dihalogenated substrate, respectively. The curves in A, B, D, E, G, and H represent the best fit of the dihalogenation mechanism parameters (see main text) to the data, while the curves in C, F, and I are best-fit single or double-exponential progress curves (here, enzyme was precipitating during reactions) by DynaFit. The experiments were performed in duplicate.

FIGS. 48A-B show the reusability of Affi-Gel®-enzyme conjugate for the halogenation assay. Chlorination of (A) compound 3, and (B) compound 11 was tested ten times (x-axis) by reusing the same Affi-Gel®-enzyme conjugate for each reaction. The reactions were repeated five times on the first day and five times again on the second day. The fractions of the substrate, mono-chlorinated product, and dichlorinated product are shown by orange, blue, and pink bars, respectively. The black dots show the overall halogenation % of each substrate. Note: The chlorination patterns shown for Cl-3, diCl-3, Cl-11, and diCl-11 were established by NMR spectroscopy.

FIG. 49 shows ¹H NMR spectrum for compound 4,6-dichlororesorcinol (4,6-diCl-3) in CD₃OD (500 MHz).

FIG. 50 shows ¹H NMR spectrum for compound 4,6-dibromoresorcinol (4,6-diBr-3) in CD₃OD (500 MHz).

FIG. 51 shows ¹³C NMR spectrum for compound 4,6-dibromoresorcinol (4,6-diBr-3) in CD₃OD (100 MHz).

FIG. 52 shows ¹H NMR spectrum for compound 2,4,6-trichlororesorcinol (4,6-diCl-8) in CD₃OD (500 MHz).

FIG. 53 shows ¹H NMR spectrum for compound 2,4-dichloro-5-methylresorcinol (2,4-diCl-9) in CD₃OD (500 MHz).

FIG. 54 shows ¹³C NMR spectrum for compound 2,4-dichloro-5-methylresorcinol (2,4-diCl-9) in CD₃OD (100 MHz).

FIG. 55 shows ¹H NMR spectrum for compound 2,6-dichloro-3,5-dihydroxybenzyl alcohol (2,6-diCl-11) in CD₃OD (500 MHz).

FIG. 56 shows ¹³C NMR spectrum for compound 2,6-dichloro-3,5-dihydroxybenzyl alcohol (2,6-diCl-11) in CD₃OD (100 MHz).

FIG. 57 shows HMBC spectrum for compound 2,6-dichloro-3,5-dihydroxybenzyl alcohol (2,6-diCl-11) in CD₃OD (100 MHz).

FIG. 58 shows ¹H NMR spectrum for compound 3,5-dichloro-2,4,6-trihydroxyacetophenone (3,5-diCl-15) in CD₃OD (500 MHz).

FIG. 59 shows ¹H NMR spectrum for compound 5-amino-2,4-dichlorophenol (2,4-diCl-16) in CD₃OD (400 MHz).

FIG. 60 shows ¹³C NMR spectrum for compound 5-amino-2,4-dichlorophenol (2,4-diCl-16) in CD₃OD (100 MHz).

FIG. 61 shows HSQC spectrum for compound 5-amino-2,4-dichlorophenol in CD₃OD (2,4-diCl-16) (100 MHz).

FIG. 62 shows HMBC spectrum for compound 5-amino-2,4-dichlorophenol (2,4-diCl-16) in CD₃OD (100 MHz).

FIG. 63 shows ¹H NMR spectrum for compound 2,4-dichloro-1,5-diaminobenzene (2,4-diCl-18) in CD₃OD (500 MHz).

FIG. 64 shows ¹³C NMR spectrum for compound 2,4-dichloro-1,5-diaminobenzene (2,4-diCl-18) in CD₃OD (100 MHz).

FIG. 65 shows ¹H NMR spectrum for compound 4-chloro-resveratrol (4-Cl-23) in CD₃OD (400 MHz).

FIG. 66 shows ¹H NMR spectrum for compound resveratrol (23) in CD₃OD (400 MHz). Commercially available resveratrol used for comparison with FIG. 65 to determine the position of Cl.

While the disclosure is susceptible to various modifications and alternative forms, specific embodiments thereof have been shown by way of example in the drawings and are herein described below in detail. It should be understood, however, that the description of specific embodiments is not intended to limit the disclosure to cover all modifications, equivalents and alternatives falling within the spirit and scope of the disclosure as defined by the appended claims.

### DETAILED DESCRIPTION

The details of one or more embodiments of the presently-disclosed subject matter are set forth in this document. Modifications to embodiments described in this document, and other embodiments, will be evident to those of ordinary skill in the art after a study of the information provided in this document. The information provided in this document, and particularly the specific details of the described exemplary embodiments, is provided primarily for clearness of understanding and no unnecessary limitations are to be understood therefrom. In case of conflict, the specification of this document, including definitions, will control.

Unless defined otherwise, all technical and scientific terms used herein have the same meaning as is commonly understood by one of skill in the art to which the invention(s) belong. All patents, patent applications, published applications and publications, GenBank sequences, databases, websites and other published materials referred to throughout the entire disclosure herein, unless noted otherwise, are incorporated by reference in their entirety. In the event that there are a plurality of definitions for terms herein, those in this section prevail. Where reference is made to a URL or other such identifier or address, it understood that such identifiers can change and particular information on the internet can come and go, but equivalent information can be found by searching the internet. Reference thereto evidences the availability and public dissemination of such information.

Following long-standing patent law convention, the terms “a”, “an”, and “the” refer to “one or more” when used in this application, including the claims. Thus, for example, reference to “a cell” includes a plurality of such cells, and so forth.

The terms “comprising,” “including,” and “having” are intended to be inclusive and mean that there may be additional elements other than the listed elements.

Unless otherwise indicated, all numbers expressing quantities of ingredients, properties such as reaction conditions, and so forth used in the specification and claims are to be understood as being modified in all instances by the term “about”. Accordingly, unless indicated to the contrary, the numerical parameters set forth in this specification and claims are approximations that can vary depending upon the desired properties sought to be obtained by the presently-disclosed subject matter.

As used herein, ranges can be expressed as from “about” one particular value, and/or to “about” another particular value. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, the term “about,” when referring to a value or to an amount of mass, weight, time, volume, concentration, percentage, or the like is meant to encompass variations of in some embodiments ±50%, in some embodiments ±40%, in some embodiments ±30%, in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed method.

All combinations of method or process steps as used herein can be performed in any order, unless otherwise specified or clearly implied to the contrary by the context in which the referenced combination is made.

As used herein, nomenclature for compounds, including organic compounds, can be given using common names, IUPAC, IUBMB, or CAS recommendations for nomenclature. When one or more stereochemical features are present, Cahn-Ingold-Prelog rules for stereochemistry can be employed to designate stereochemical priority, ElZ specification, and the like. One of skill in the art can readily ascertain the structure of a compound if given a name, either by systemic reduction of the compound structure using naming conventions, or by commercially available software, such as CHEMDRAW™ (Cambridgesoft Corporation, U.S.A.).

As used herein, the terms “optional” or “optionally” means that the subsequently described event or circumstance can or cannot occur, and that the description includes instances where said event or circumstance occurs and instances where it does not.

As used herein, the term “subject” can be a vertebrate, such as a mammal, a fish, a bird, a reptile, or an amphibian. Thus, the subject of the herein disclosed methods can be a human, non-human primate, horse, pig, rabbit, dog, sheep, goat, cow, cat, guinea pig or rodent. The term does not denote a particular age or sex. Thus, adult and newborn subjects, as well as fetuses, whether male or female, are intended to be covered. In one aspect, the subject is a mammal. A patient refers to a subject afflicted with a disease or disorder. The term “patient” includes human and veterinary subjects.

As used herein, the term “derivative” refers to a compound having a structure derived from the structure of a parent compound (e.g., a compound disclosed herein) and whose structure is sufficiently similar to those disclosed herein and based upon that similarity, would be expected by one skilled in the art to exhibit the same or similar activities and utilities as the claimed compounds, or to induce, as a precursor, the same or similar activities and utilities as the claimed compounds. Exemplary derivatives include salts, esters, amides, salts of esters or amides, and N-oxides of a parent compound.

Although any methods, devices, and materials similar or equivalent to those described herein can be used in the practice or testing of the presently-disclosed subject matter, representative methods, devices, and materials are now described.

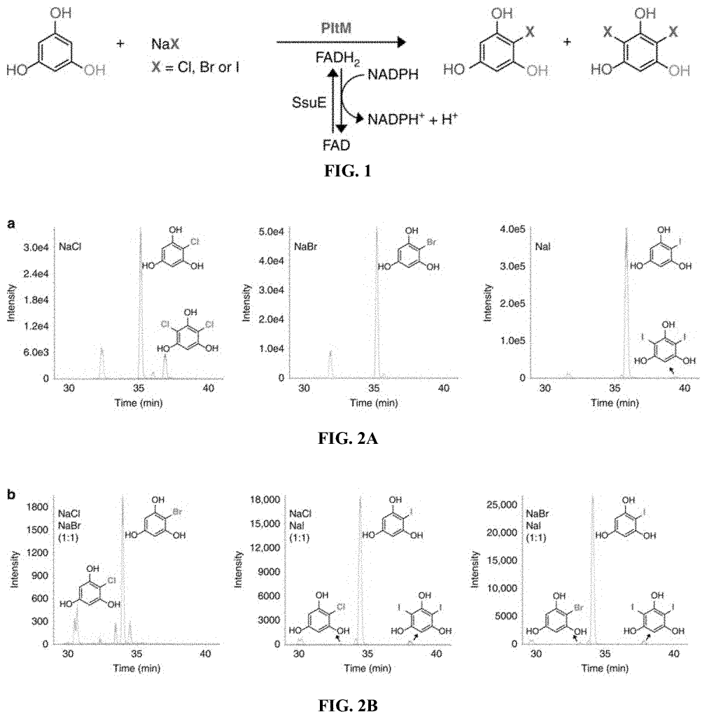

The presently-disclosed subject matter relates to a halogenation system. In some embodiments, the halogenation system includes a bacterial halogenase. Suitable bacterial halogenases include, but are not limited to, PltM. PltM is encoded in the biosynthetic gene cluster of pyoluteorin, an antifungal compound containing a dichloropyrrole moiety. In some embodiments, PltM halogenates substrates with one or more halides, such as, but not limited to, Cl⁻, Br⁻, I⁻, or a combination thereof. This halogenation of the substrate by PltM may include mono-halogenation or di-halogenation with the same or different halogens. For example, in one embodiment, as illustrated in FIG. 1 and Table 2, PltM catalyzes mono- and dichlorination of phloroglucinol (1). In another embodiment, instead of a biosynthetic intermediate, PltM catalyzed chlorination yields a compound that serves as a potent transcriptional regulator of the pyolyteorin biosynthesis. This is also in contrast to PltA, another halogenase which acts on a peptidyl carrier protein loaded pyrrole to generate dichloropyrrole.

TABLE 2

| Main | | Halogenation | |
|---|---|---|---|
| substrate | Halogenase | position | PDB codes* |
| Phloroglucinol | PltM | C2 or C2 | 6BZN (apo) |
| | | and C4 | 6BZA (phloroglucinol |
| | | | and partially bound |
| | | | FAD) |
| | | | 6BZQ (FAD) |
| | | | 6BZZ (FAD-partially |
| | | | bound) |
| | | | 6BZT (L111Y, FAD) |

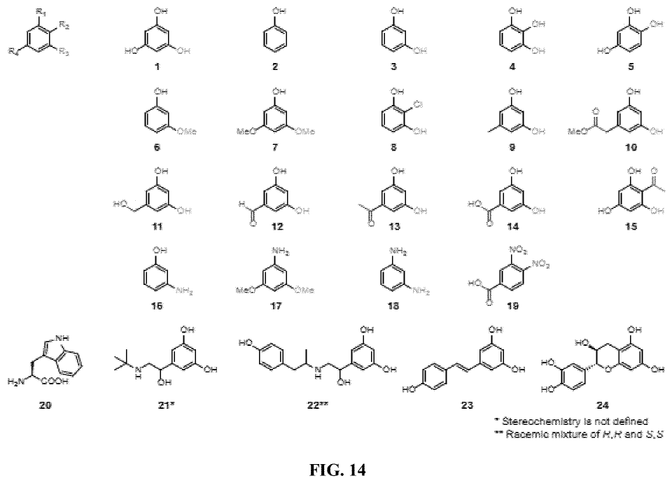

Although discussed above with regard to chlorination of phloroglucinol, the disclosure is not so limited and includes halogenation of other substrates with the same or different halides. In some embodiments, the substrate includes phenyl compounds with electron donating groups. In one embodiment, such compounds include, but are not limited to, phenolic derivatives (e.g., FIG. 14—compounds 1-16), aniline derivatives (e.g., FIG. 14—compounds 16-18), or a combination thereof. In another embodiment, the substrates may include compounds where one hydroxyl group has been substituted with a moderate electron withdrawing group, such as, but not limited to, aldehyde (e.g., FIG. 14—compound 12), ketone (e.g., FIG. 14—compound 13), or carboxylic acid (e.g., FIG. 14—compound 14). Additionally or alternatively, in some embodiments, the substrate includes larger molecules and/or natural products. In one embodiment, the larger molecules include compounds having a resorcinol moiety in their structure. For example, the larger molecules may include short-acting b2 adrenoreceptor agonists, such as, but not limited to, terbutaline (FIG. 14—compound 21) and/or fenoterol (FIG. 14—compound 22). In one embodiment, the natural products include dietary natural products. For example, the natural products may include resveratrol (FIG. 14—compound 23) and/or catechin (FIG. 14—compound 24). Any of these substrates may be mono- or di-hologenated with one or more of the halides disclosed herein.

In some embodiments, the halogenation system includes multiple enzymes. In one embodiment, the system includes PltM and at least one other enzyme. In another embodiment, the at least one other enzyme includes one or more of a NADPH regenerator, such as glucose dehydrogenase (GDH), or a flavin adenine dinucleotide (FAD) reductase, such as SsuE. In some embodiments, the enzymes are immobilized on a solid support. Suitable solid supports include, but are not limited to, resins, such as the agarose resin Affi-Gel® 15. For example, in one embodiment, the halogenation system includes PltM, SsuE, and GDH immobilized on agarose resin (Affi-Gel® 15). In some embodiments, the immobilized enzymes are packed into a spin column, which may be used as a resin conjugate for halogenation. This protein bound resin provides a high halogenation yield for some compounds, which could not be efficiently halogenated by free enzymes in solution. Additionally or alternatively, the enzyme-resin conjugate may be reused 5-6 times without significant loss of efficiency. Without wishing to be bound by theory, this reusability is believed to be the result of a unique recycling mechanism of FAD provided by the combination of immobilized enzymes.

Also provided herein are methods of using the halogenation system. In some embodiments, the methods include running a substrate and reaction solution through the halogenation system disclosed herein. Any suitable substrate may be used based upon the one or more enzymes within the halogenation system. Suitable substrates include, but are not limited to, phenolic derivatives (e.g., FIG. 14—compounds 1-16), aniline derivatives (e.g., FIG. 14—compounds 16-18), short-acting b2 adrenoreceptor agonists (e.g., terbutaline and/or fenoterol; FIG. 14—compounds 21-22), natural products (e.g., resveratrol and/or catechin; FIG. 14—compounds 23-24), or a combination thereof, The one or more enzymes within the halogenation system interact with the substrate as it is run through the system, modifying the substrate as it passes therethrough. For example, in some embodiments, the system may be used to modify biologically active molecules (including those currently in clinical use) to create new chemical entities with improved medicinal properties. Additionally or alternatively, in some embodiments, the halogenation system allows medicinal chemists to access a previously unaccessible or difficult to access regions of chemical space. Furthermore, at least ⅓ of currently prescribed drugs are believed to be substrates of this enzymatic system, which could modify them to improve their current properties.

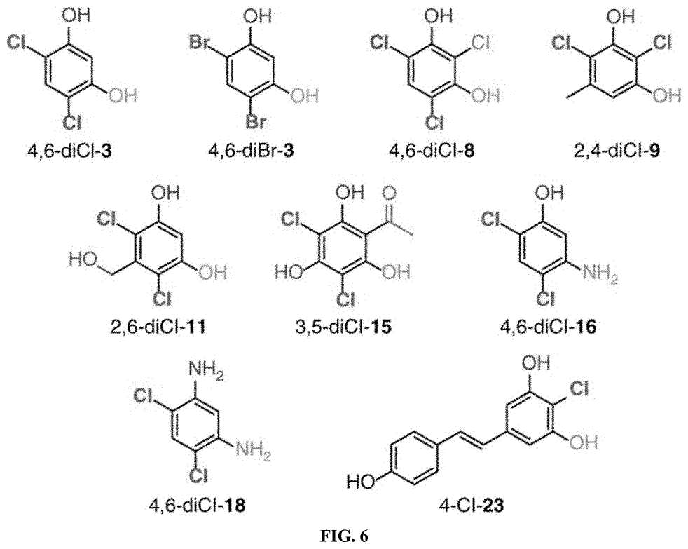

Also provided herein are halogenated compounds formed with the halogenation system. The compounds include mono- and di-halogenated derivatives of any suitable PltM substrate. In one embodiment, the mono-halogenated derivatives include mono-chlorinated derivatives such as, but not limited to, 4-Cl-23 (FIG. 6). In one embodiment, the di-halogenated derivatives include di-chlorinated derivatives such as, but not limited to, 4,6-diCl-3, 4,6-diCl-8, 2,4-diCl-9, 2,6-diCl-11, 3,5-diCl-15, 4,6-diCl-16, 4,6-diCl-18, and/or 4-Cl-23 (FIG. 6). In one embodiment, the di-halogenated derivatives include di-brominated derivatives such as, but not limited to, 4,6-diBr-3 (FIG. 6). As will be appreciated by those skilled in the art, the mono- and di-halogenated derivatives are not limited to the examples above and may include any other suitable mono-chlorinated, mono-brominated, mono-iodinated, di-chlorinated, di-brominated, di-iodinated, and/or hetero-di-halogenated compound.

The presently-disclosed subject matter is further illustrated by the following specific but non-limiting examples. The following examples may include compilations of data that are representative of data gathered at various times during the course of development and experimentation related to the presently-disclosed subject matter. Those skilled in the art will recognize, or be able to ascertain, using no more than routine experimentation, numerous equivalents to the specific substances and procedures described herein.

### EXAMPLES

### Example 1

This Example describes the characterization PltM and exploration of its ability to halogenate various compounds.

Results

Halide Versatility of PltM

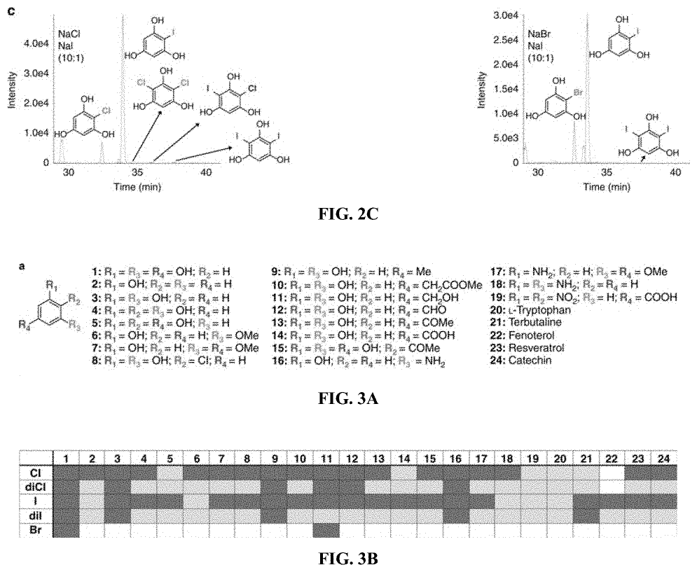

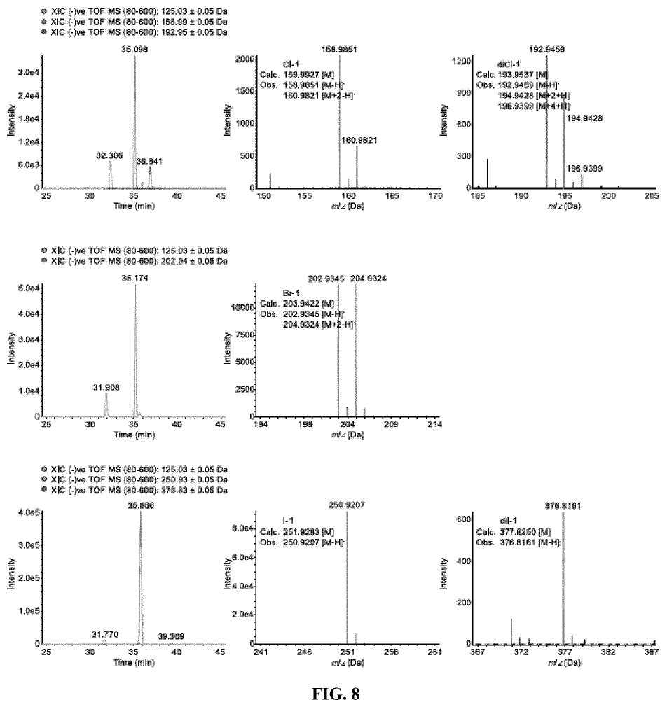

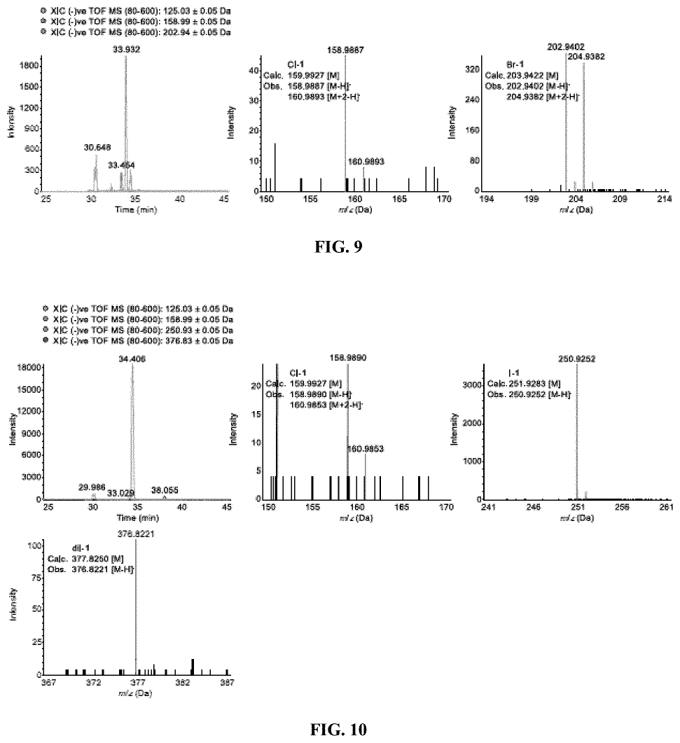

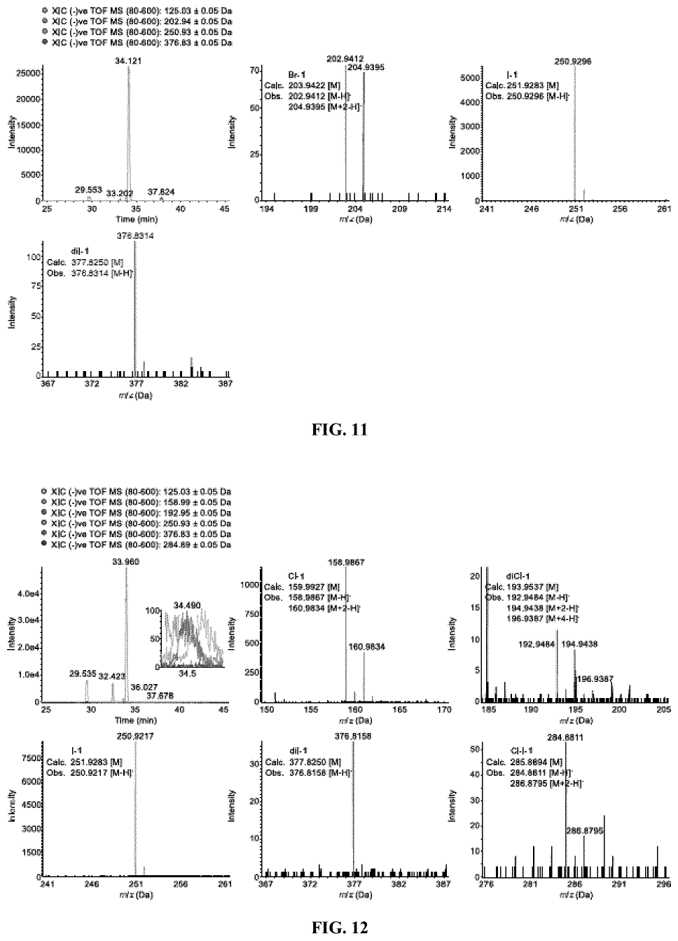

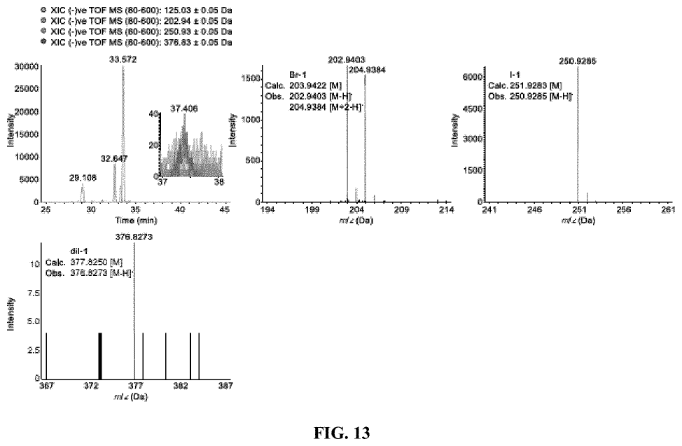

To explore the halide profile of PltM, halogenation of 1 by PltM with NaF, NaCl, NaBr, and NaI used individually in a reaction mixture was tested first. Chlorinated, brominated, and iodinated, but not fluorinated 1, were identified as products (FIGS. 2A and 8; Table 3). Without wishing to be bound by theory, it is believed that PltM is the only example of an FAD-dependent halogenase that is able to use three different halides, Cl⁻, Br⁻, and I⁻. Mono- and dihalogenation of 1 was observed with chloride and iodide, but only mono-halogenation was observed with bromide; trihalogenation was never observed. Competitive halogenation assays of 1 were carried out next, where two different halides (Cl⁻/Br⁻, Cl⁻/I⁻, or Br⁻/I⁻) were present in the reaction at equimolar ratios (FIGS. 2B and 9-11; Table 3). Each of these reactions yielded mono-halogenated products of either halogen and diiodinated 1 where NaI was used, whereas products halogenated by two different halides were not observed. In an attempt to obtain a hetero-dihalogenated product, compound 1 was used in the presence of a 10-fold molar excess of NaCl or NaBr over NaI (FIGS. 2C and 12-13; Table 3). For the NaCl/NaI mixture, all possible mono- and dihalogenated products were identified, including chloro-iodinated 1. For NaBr/NaI, mono-brominated, mono-iodinated, and diiodinated 1 were identified, and no additional products were observed. In fact, no further halogenation of the mono-brominated species was observed in any reaction.

TABLE 3
LC/MS data for Assay 1 and Assay 2 against phloroglucinol (1)

| | | | Obs. | Obs. | Obs. | | | |
|---|---|---|---|---|---|---|---|---|
| | | Calcd. | mass | mass | mass | Retention | | |
| | | mass | [M − H]⁻ | [M + 2 − H]⁻ | [M + 4 − H]⁻ | time | Assay | Fig. |
| Substrate | Product | (Da) | (Da) | (Da) | (Da) | (min) | # | # |
| 1 | 1 | 126.0317 | 125.0244 | — | — | 32.306 | Std | 1b, S1, S8 |
| | F-1 | 144.0223 | — | — | — | — | 1a | — |
| | diF-1 | 162.0129 | — | — | — | — | 1a | — |
| | Cl-1 | 159.9927 | 158.9851 | 160.9821 | — | 35.098 | 1b | 1b, S1 |
| | diCl-1 | 193.9537 | 192.9459 | 194.9428 | 196.9399 | 36.841 | 1b | 1b, S1 |
| | Br-1 | 203.9422 | 202.9345 | 204.9324 | — | 35.174 | 1c | 1b, S1 |
| | diBr-1 | 283.8527 | — | — | — | — | 1c | — |
| | I-1 | 251.9283 | 250.9207 | — | — | 35.866 | 1d | 1b, S1 |
| | diI-1 | 377.8250 | 376.8161 | — | — | 39.309 | 1d | 1b, S1 |
| 1 | Cl-1 | 159.9927 | 158.9887 | 160.9893 | — | 33.454 | 2a | 1c, S2 |
| | diCl-1 | 193.9537 | — | — | — | — | 2a | — |
| | Br-1 | 203.9422 | 202.9402 | 204.9382 | — | 33.932 | 2a | 1c, S2 |
| | diBr-1 | 283.8527 | — | — | — | — | 2a | — |
| | Cl,Br-1 | 237.9032 | — | — | — | — | 2a | — |
| | Cl-1 | 159.9927 | 158.9890 | 160.9853 | — | 33.029 | 2b | 1c, S3 |
| | diCl-1 | 193.9537 | — | — | — | — | 2b | — |
| | I-1 | 251.9283 | 250.9252 | — | — | 34.406 | 2b | 1c, S3 |
| | diI-1 | 377.8250 | 376.8221 | — | — | 38.055 | 2b | 1c, S3 |
| | Cl,I-1 | 285.8894 | — | — | — | — | 2b | — |
| | Br-1 | 203.9422 | 202.9412 | 204.9395 | — | 33.202 | 2c | 1c, S4 |
| | diBr-1 | 283.8527 | — | — | — | — | 2c | — |
| | I-1 | 251.9283 | 250.9296 | — | — | 34.121 | 2c | 1c, S4 |
| | diI-1 | 377.8250 | 376.8314 | — | — | 37.824 | 2c | 1c, S4 |
| | Br,I-1 | 329.8388 | — | — | — | — | 2c | — |
| | Cl-1 | 159.9927 | 158.9867 | 160.9834 | — | 32.423 | 2d | 1d, S5 |
| | diCl-1 | 193.9537 | 192.9484 | 194.9438 | 196.9387 | 34.490 | 2d | 1d, S5 |
| | I-1 | 251.9283 | 250.9217 | — | — | 33.960 | 2d | 1d, S5 |
| | diI-1 | 377.8250 | 376.8158 | — | — | 37.678 | 2d | 1d, S5 |
| | Cl,I-1 | 285.8894 | 284.8811 | 286.8795 | — | 36.027 | 2d | 1d, S5 |
| | Br-1 | 203.9422 | 202.9403 | 204.9384 | — | 32.647 | 2e | 1d, S6 |
| | diBr-1 | 283.8527 | — | — | — | — | 2e | — |
| | I-1 | 251.9283 | 250.9285 | — | — | 33.572 | 2e | 1d, S6 |
| | diI-1 | 377.8250 | 376.8273 | — | — | 37.406 | 2e | — |
| | Br,I-1 | 329.8388 | — | — | — | — | 2e | — |

Note:
Although we looked for trihalogenated 1, we did not observe any. All masses were measured in negative mode.

Substrate Profile of PltM

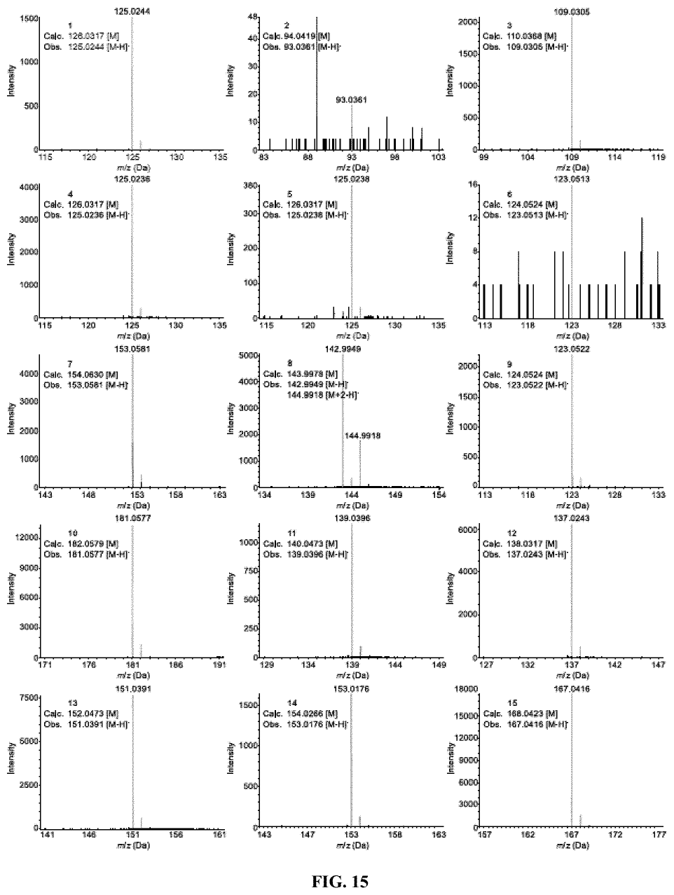

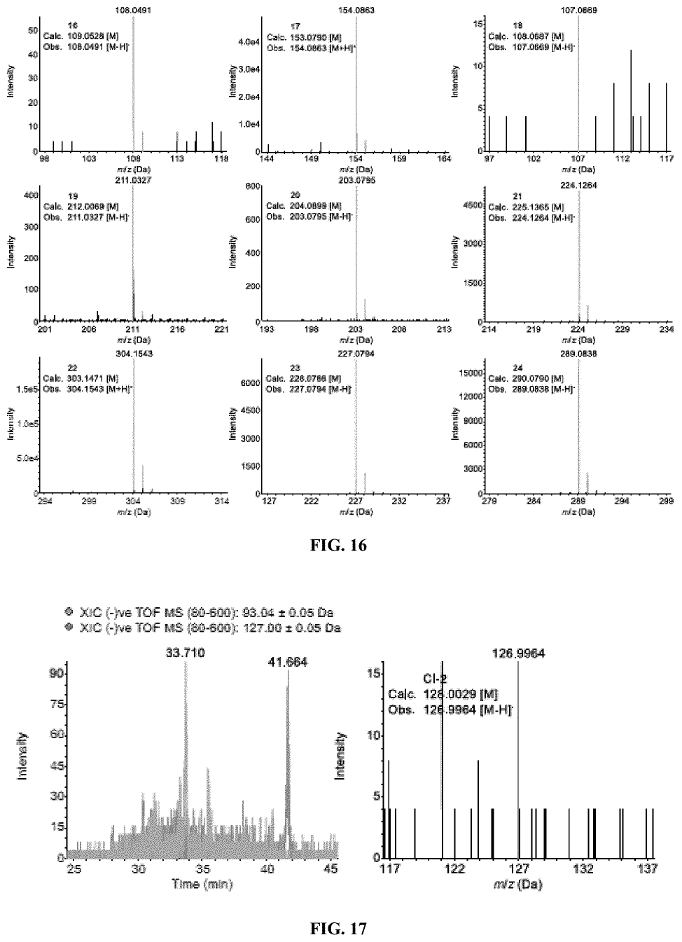

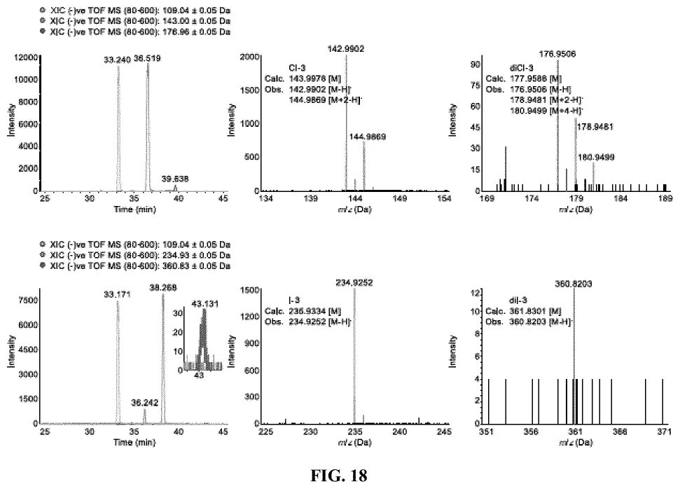

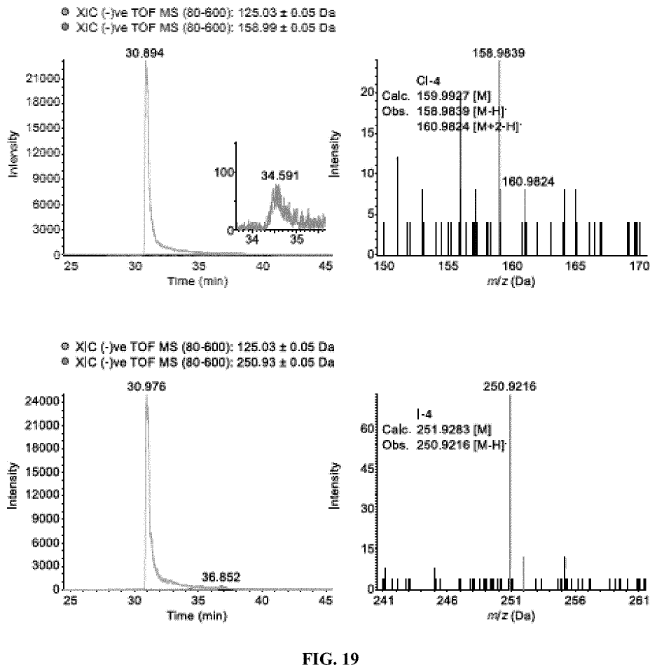

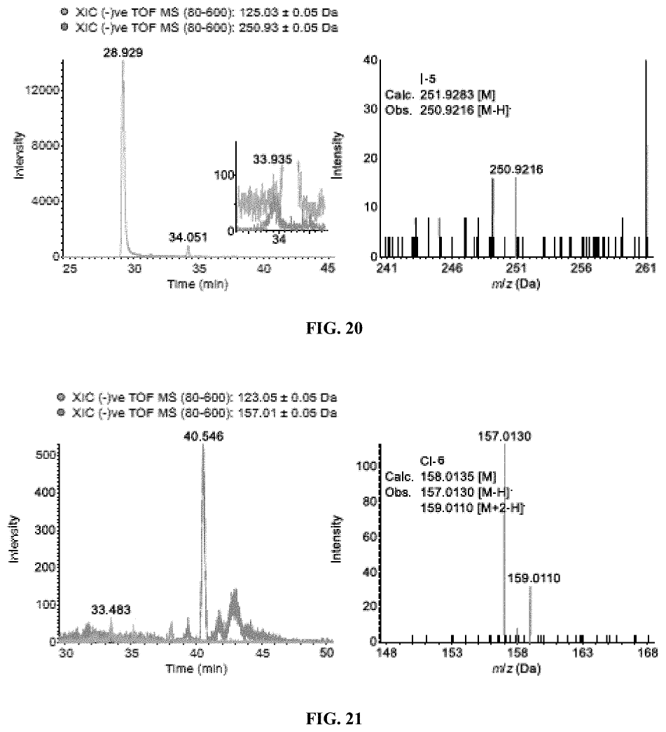

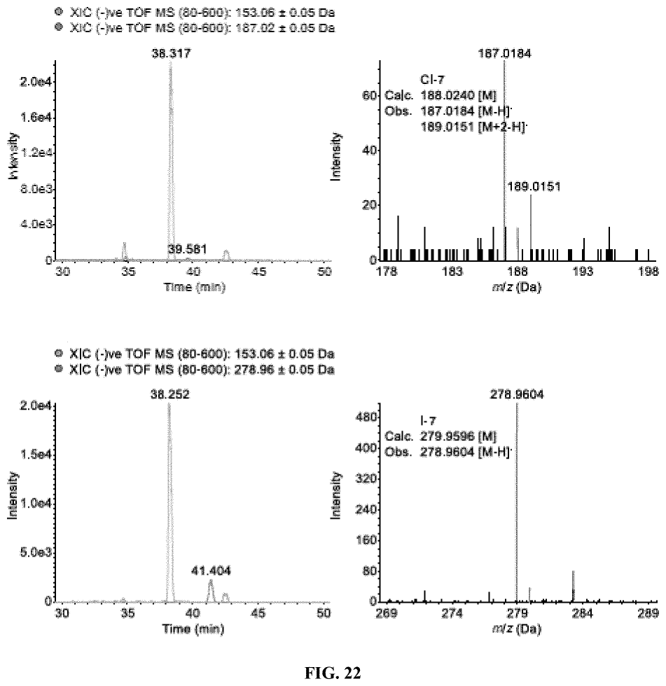

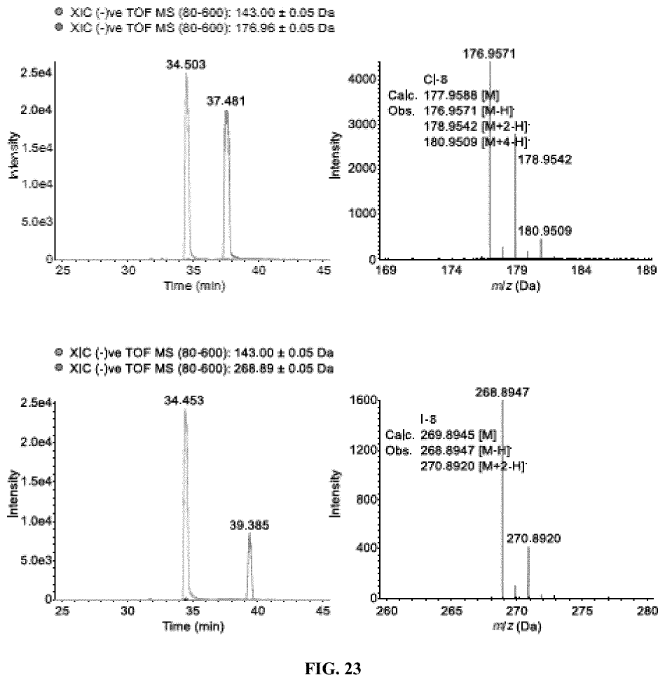

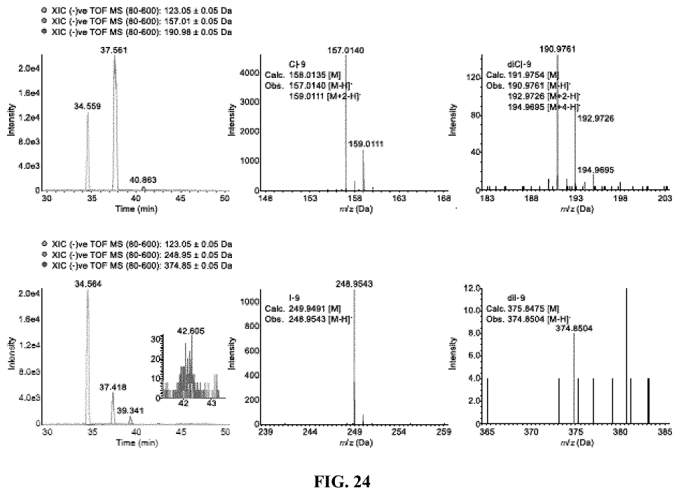

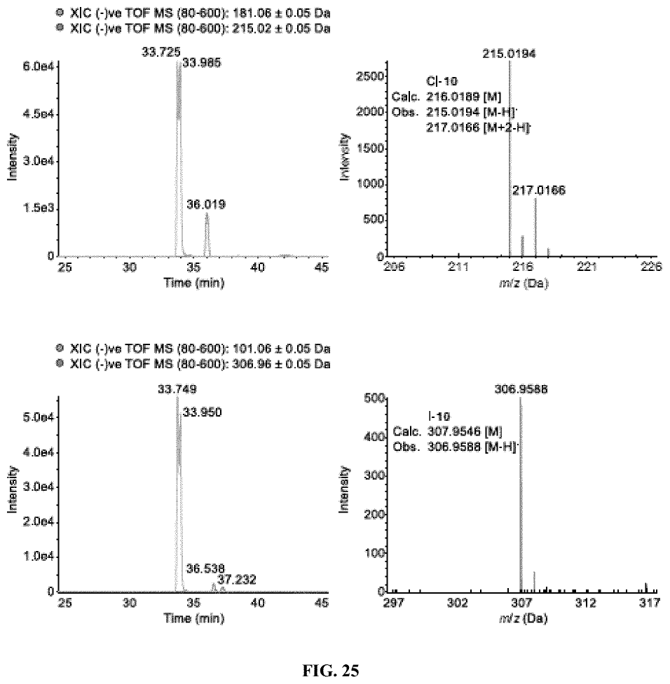

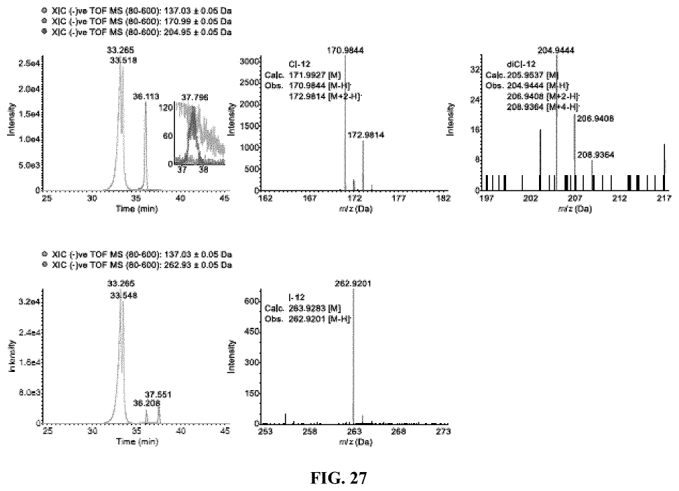

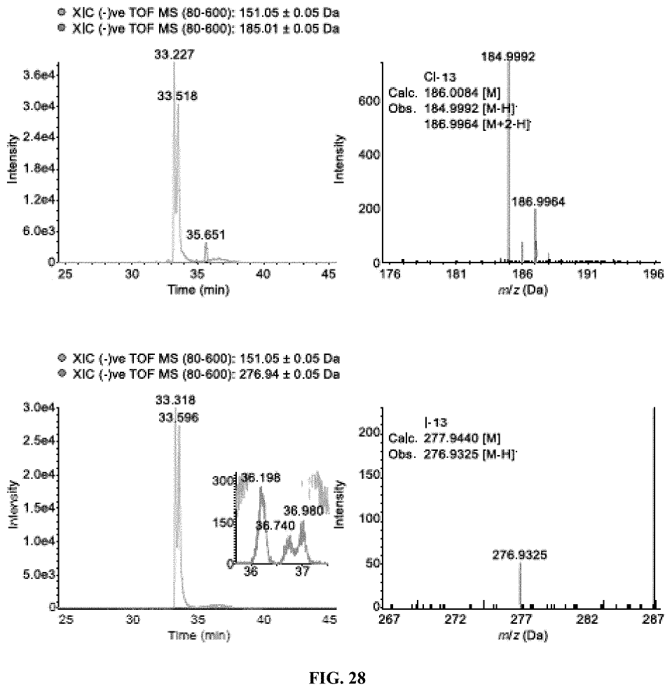

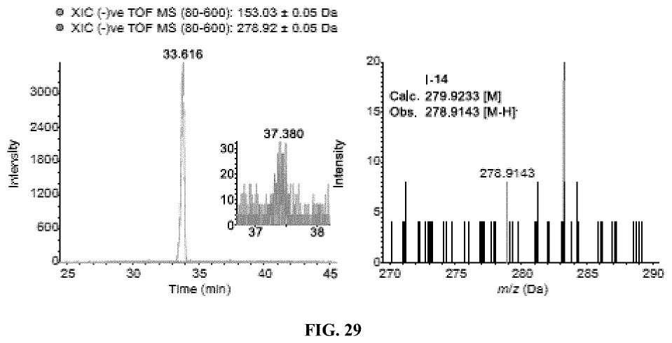

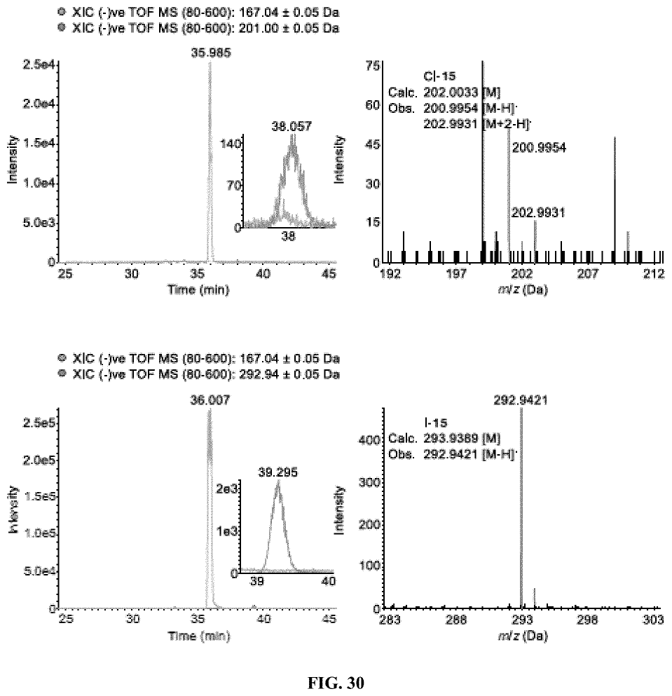

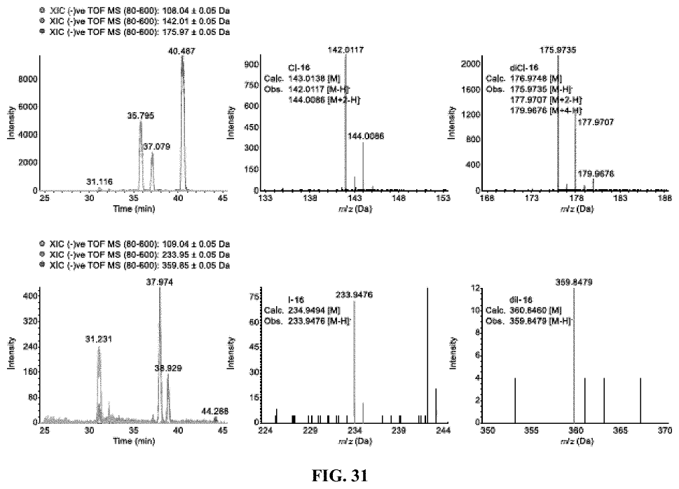

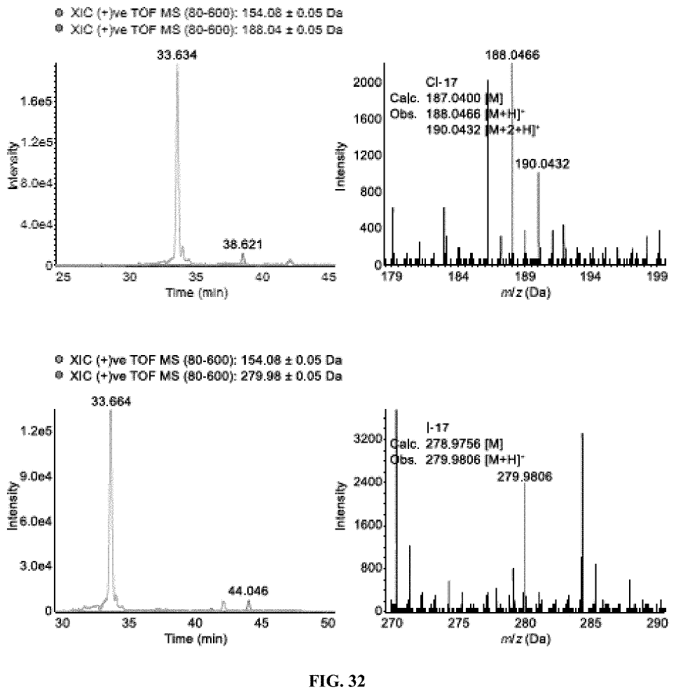

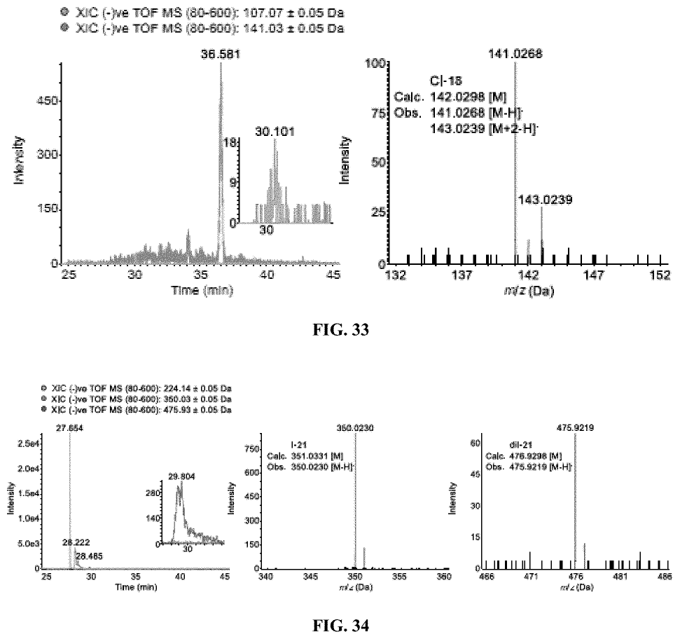

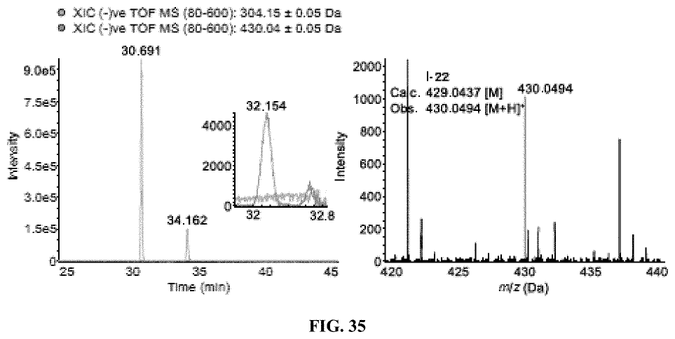

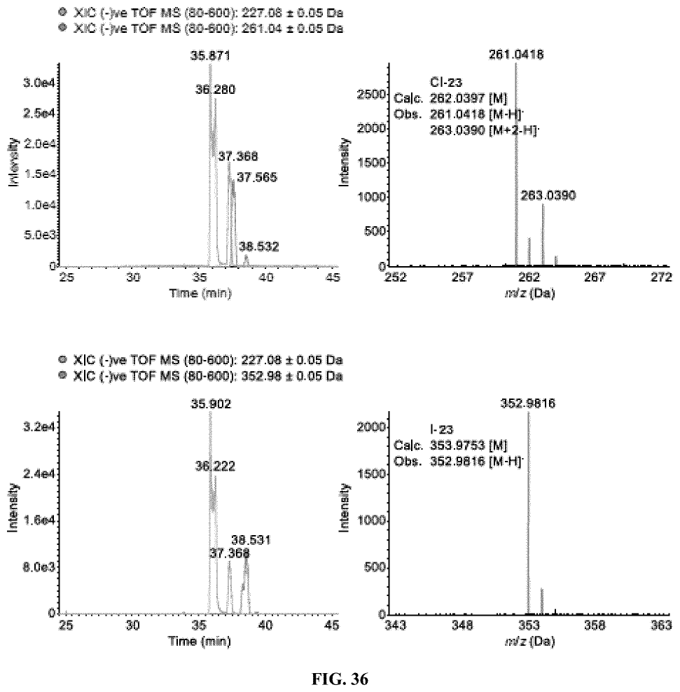

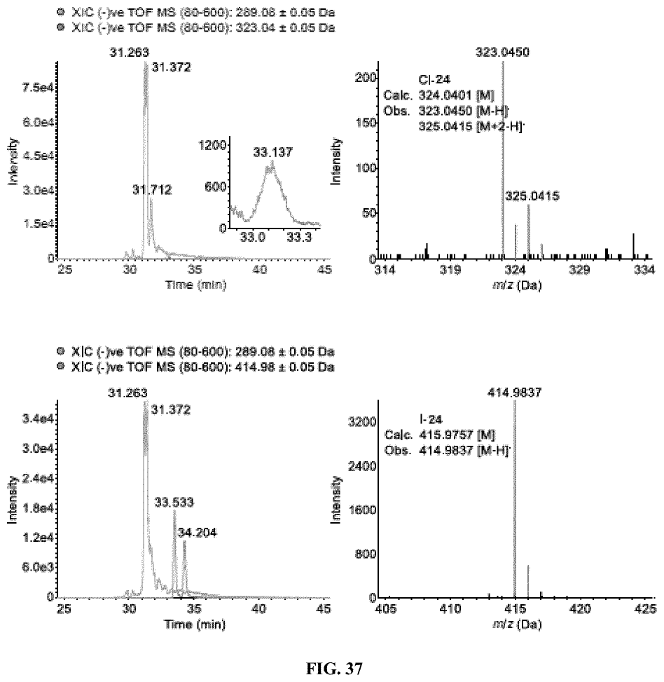

Having established the halide versatility of PltM, its substrate profile was investigated next. A set of 20 structurally diverse small molecules was tested first, most, but not all of which were, like 1, phenolic (phenolic derivatives, anilines, nitrobenzene derivative) and included L-Trp (FIGS. 3A and 14). All compounds were tested for chlorination and iodination (FIGS. 3B and 15-37; Table 4). The products were detected and identified by liquid chromatography-mass spectrometry (LC-MS); chlorinated products were identified by the calculated mass and isotope ratio, and iodinated products were identified by the calculated mass, also using the corresponding chlorination reaction as a control.

PltM catalyzed halogenation of 18 of the 20 compounds tested, exhibiting remarkable substrate versatility for phenolic compounds (FIG. 3B). The enzyme halogenated all phenolic (1-16) and aniline (16-18) derivatives tested, while it did not halogenate the nitrobenzene derivative 19. These data suggest that the phenyl compounds with electron donating groups can be accepted by PltM as substrates even when one hydroxyl group is substituted with a moderate electron withdrawing group, such as aldehyde (12), ketone (13), and carboxylic acid (14). On the other hand, the strongly electron withdrawing nitro group is not tolerated. This correlation of the substrate electron withdrawing character with halogenation activity is consistent with other phenolic halogenases. The halogenated L-Trp (20) was not observed, indicating that PltM is not a Trp halogenase, and that it is indeed a bona fide phenolic halogenase.

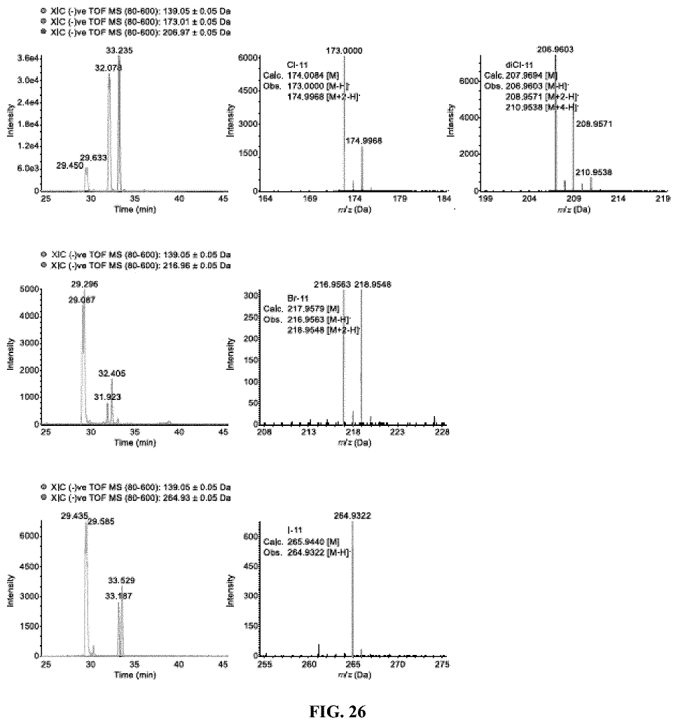

Since the reaction with compound 11 showed very clear signals of chlorinated and iodinated 11, it was also tested for bromination and fluorination (FIG. 26; Table 4). Mono-bromination of 11 was observed, but not dibromination or fluorination, which is consistent with the halogenation profile on the natural substrate 1. Encouraged by the wide substrate versatility of PltM as established with compounds 1-20, its halogenase activity was tested on four larger molecules containing a phenolic derivative group. The FDA-approved drugs terbutaline (21) and fenoterol (22) were tested, both of which are short-acting b2 adrenoreceptor agonists that contain a resorcinol moiety in their structure (FIG. 14). Iodinated terbutaline (both mono and di) and mono-iodinated fenoterol were obtained (FIG. 3B). The dietary natural products resveratrol (23) and catechin (24) were also tested, which were both mono-chlorinated and mono-iodinated by PltM. These results demonstrate that PltM can be utilized for halogenation of larger drug-like molecules and natural products.

TABLE 4
LC/MS data for all tested substrates.

| | | | Obs. | Obs. | Obs. | | | |
|---|---|---|---|---|---|---|---|---|
| | | | mass | mass | mass | | | |
| | | Calcd. | [M − H]⁻/ | [M + 2 − H]⁻/ | [M + 4 − H]⁻/ | Retention | | |
| | | mass | [M + H]⁻ | [M + 2 + H]⁺ | [M + 4 + H]⁺ | time | Assay | Fig. |
| Substrate | Product | (Da) | (Da) | (Da) | (Da) | (min) | # | # |
| 2 | 2 | 94.0419 | 93.0361 | — | — | 33.710 | Std. | S8, S10 |
| | Cl-2 | 128.0029 | 126.9964 | — | — | 41.664 | 1b | S10 |
| | diCl-2 | 161.9639 | — | — | — | — | 1b | — |
| | I-2 | 219.9385 | — | — | — | — | 1d | — |
| | diI-2 | 345.8352 | — | — | — | — | 1d | — |
| 3 | 3 | 110.0368 | 109.0305 | — | — | 33.240 | Std. | S8, S11 |
| | Cl-3 | 143.9978 | 142.9902 | 144.9869 | — | 36.519 | 1b | S11 |
| | diCl-3 | 177.9588 | 176.9506 | 178.9481 | 180.9499 | 39.638 | 1b | S11 |
| | I-3 | 235.9334 | 234.9252 | — | — | 36.242/38.268 | 1d | S11 |
| | diI-3 | 361.8301 | 360.8203 | — | — | 43.131 | 1d | S11 |
| 4 | 4 | 126.0317 | 125.0236 | — | — | 30.894 | Std. | S8, S12 |
| | Cl-4 | 159.9927 | 158.9839 | 160.9824 | — | 34.591 | 1b | S12 |
| | diCl-4 | 193.9537 | — | — | — | — | 1b | — |
| | I-4 | 251.9283 | 250.9216 | — | — | 36.852 | 1d | S12 |
| | diI-4 | 377.8250 | — | — | — | — | 1d | — |
| 5 | 5 | 126.0317 | 125.0238 | — | — | 28.929/34.051 | Std. | S8, S13 |
| | Cl-5 | 159.9927 | — | — | — | — | 1b | — |
| | diCl-5 | 193.9537 | — | — | — | — | 1b | — |
| | I-5 | 251.9283 | 250.9216 | — | — | 33.935 | 1d | S13 |
| | diI-5 | 377.8250 | — | — | — | — | 1d | — |
| 6 | 6 | 124.0524 | 123.0513 | — | — | 33.483 | Std. | S8, S14 |
| | Cl-6 | 158.0135 | 157.0130 | 159.0110 | — | 40.546 | 1b | S14 |
| | diCl-6 | 191.9745 | — | — | — | — | 1b | — |
| | I-6 | 249.9491 | — | — | — | — | 1d | — |
| | diI-6 | 375.8457 | — | — | — | — | 1d | — |
| 7 | 7 | 154.0630 | 153.0581 | — | — | 38.317 | Std. | S8, S15 |
| | Cl-7 | 188.0240 | 187.0184 | 189.0151 | — | 39.581 | 1b | S15 |
| | diCl-7 | 221.9850 | — | — | — | — | 1b | — |
| | I-7 | 279.9596 | 278.9604 | — | — | 41.04 | 1d | S15 |
| | diI-7 | 405.8563 | — | — | — | — | 1d | — |
| 8 | 8 | 143.9978 | 142.9949 | 144.9918 | — | 34.503 | Std. | S8, S16 |
| | Cl-8 | 177.9588 | 176.9571 | 178.9542 | 180.9509 | 37.481 | 1b | S16 |
| | I-8 | 269.8945 | 268.8947 | 270.8920 | — | 39.385 | 1b | S16 |
| 9 | 9 | 124.0524 | 123.0522 | — | — | 34.559 | Std. | S8, S17 |
| | Cl-9 | 158.0135 | 157.0140 | 159.0111 | — | 37.561 | 1b | S17 |
| | diCl-9 | 191.9754 | 190.9761 | 192.9726 | 194.9695 | 40.863 | 1b | S17 |
| | I-9 | 249.9491 | 248.9543 | — | — | 37.418/39.341 | 1d | S17 |
| | diI-9 | 375.8457 | 374.8504 | — | — | 42.605 | 1d | S17 |
| 10 | 10 | 182.0579 | 181.0577 | — | — | 33.725/33.985 | Std. | S8, S18 |
| | Cl-10 | 216.0189 | 215.0194 | 217.0166 | — | 36.019 | 1b | S18 |
| | diCl-10 | 249.9800 | — | — | — | — | 1b | — |
| | I-10 | 307.9546 | 306.9588 | — | — | 36.538/37.232 | 1d | S18 |
| | diI-10 | 433.8512 | — | — | — | — | 1d | — |
| 11 | 11 | 140.0473 | 139.0396 | — | — | 29.450/29.633 | Std. | S8, S19 |
| | F-11 | 158.0379 | — | — | — | — | 1a | — |
| | diF-11 | 176.0285 | — | — | — | — | 1a | — |
| | Cl-11 | 174.0084 | 173.0000 | 174.9968 | | 32.078 | 1b | S19 |
| | diCl-11 | 207.9694 | 206.9603 | 208.9571 | 210.9538 | 33.235 | 1b | S19 |
| | Br-11 | 217.9579 | 216.9563 | 218.9548 | — | 31.923/32.405 | 1c | S19 |
| | DiBr-11 | 295.8684 | — | — | — | — | 1c | — |
| | I-11 | 265.9440 | 264.9321 | — | — | 33.187/33.529 | 1d | S19 |
| | diI-11 | 391.8406 | — | — | — | — | 1d | — |
| 12 | 12 | 138.0317 | 137.0243 | — | — | 33.265/33.518 | Std. | S8, S20 |
| | Cl-12 | 171.9927 | 170.9844 | 172.9814 | — | 36.113 | 1b | S20 |
| | diCl-12 | 205.9537 | 204.9444 | 206.9408 | 208.9364 | 37.796 | 1b | S20 |
| | I-12 | 263.9283 | 262.9201 | — | — | 36.208/37.551 | 1d | S20 |
| | diI-12 | 389.8250 | — | — | — | — | 1d | — |
| 13 | 13 | 152.0473 | 151.0391 | — | — | 33.227/33.518 | Std. | S8, S21 |
| | Cl-13 | 186.0084 | 184.9992 | 186.9964 | — | 35.651 | 1b | S21 |
| | diCl-13 | 219.9694 | — | — | — | — | 1b | — |
| | I-13 | 277.9440 | 276.9325 | — | — | 36.198/36.740/36.980 | 1d | S21 |
| | diI-13 | 403.8406 | — | — | — | — | 1d | — |
| 14 | 14 | 154.0266 | 153.0176 | — | — | 33.616 | Std. | S8, S22 |
| | Cl-14 | 187.9876 | — | — | — | — | 1b | — |
| | diCl-14 | 221.9487 | — | — | — | — | 1b | — |
| | I-14 | 279.9233 | 278.9143 | — | — | 37.380 | 1d | S22 |
| | diI-14 | 405.8199 | — | — | — | — | 1d | — |
| 15 | 15 | 168.0423 | 167.0416 | — | — | 35.985 | Std. | S8, S23 |
| | Cl-15 | 202.0033 | 200.9954 | 202.9931 | — | 98.057 | 1b | S23 |
| | diCl-15 | 235.9643 | — | — | — | — | 1b | — |
| | I-15 | 293.9389 | 292.9421 | — | — | 39.295 | 1d | S23 |
| | diI-15 | 419.8355 | — | — | — | — | 1d | — |
| 16 | 16 | 109.0528 | 108.0491 | — | — | 31.116 | Std. | S8, S24 |
| | Cl-16 | 143.0138 | 142.0117 | 144.0086 | — | 35.795/37.079 | 1b | S24 |
| | diCl-16 | 176.9748 | 175.9735 | 177.9707 | 179.9676 | 40.487 | 1b | S24 |
| | I-16 | 234.9494 | 233.9476 | — | — | 37.974/38.929 | 1d | S24 |
| | diI-16 | 360.8460 | 359.8479 | — | — | 44.288 | 1d | S24 |
| 17 | 17 | 153.0790 | 154.0863 | — | — | 33.634 | Std. | S8, S25 |
| | Cl-17 | 187.0400 | 188.0466 | 190.0432 | — | 38.621 | 1b | S25 |
| | diCl-17 | 211.0010 | — | — | — | — | 1b | — |
| | I-17 | 278.9756 | 279.9806 | — | — | 44.046 | 1d | S25 |
| | diI-17 | 404.8723 | — | — | — | — | 1d | — |
| 18 | 18 | 108.0687 | 107.0669 | — | — | 30.101 | Std. | S8, S26 |
| | Cl-18 | 142.0298 | 141.0268 | 143.0239 | — | 36.581 | 1b | S26 |
| | diCl-18 | 175.9908 | — | — | — | — | 1b | — |
| | I-18 | 233.9654 | — | — | — | — | 1d | — |
| | diI-18 | 359.8620 | — | — | — | — | 1d | — |
| 19 | 19 | 212.0069 | 211.0327 | | | 35.780 | Std. | S9 |
| | Cl-19 | 245.9680 | — | | | — | 1b | — |
| | diCl-19 | 279.9290 | — | | | — | 1b | — |
| | I-19 | 337.9036 | — | | | — | 1d | — |
| | diI-19 | 463.8002 | — | | | — | 1d | — |
| 20 | 20 | 204.0899 | 203.0795 | | | 29.860 | Std. | S9 |
| | Cl-20 | 238.0509 | — | | | — | 1b | — |
| | diCl-20 | 272.0119 | — | | | — | 1b | — |
| | I-20 | 329.9865 | — | | | — | 1d | — |
| | diI-20 | 455.8832 | — | | | — | 1d | — |
| 21 | 21 | 225.1365 | 224.1264 | — | — | 27.654 | Std. | S8, S27 |
| | Cl-21 | 259.0975 | — | — | — | — | 1b | — |
| | diCl-21 | 293.0585 | — | — | — | — | 1b | — |
| | I-21 | 351.0331 | 350.0230 | — | — | 28.222/28.485 | 1d | S27 |
| | diI-21 | 476.9298 | 475.9219 | — | — | 29.804 | 1d | S27 |
| 22 | 22 | 303.1471 | 304.1543 | — | — | 30.691/34.162 | Std. | S8, S28 |
| | Cl-22 | 337.1081 | — | — | — | | 1b | — |
| | diCl-22 | 371.0691 | — | — | — | | 1b | — |
| | I-22 | 429.0437 | 430.0494 | — | — | 32.154 | 1d | S28 |
| | diI-22 | 554.9403 | — | — | — | — | 1d | — |
| 23 | 23 | 228.0786 | 227.0794 | — | — | 35.871/36.280/37.368 | Std. | S8, S29 |
| | Cl-23 | 262.0397 | 261.0418 | 263.0390 | — | 37.565/38.532 | 1b | S29 |
| | diCl-23 | 296.0007 | — | — | — | — | 1b | — |
| | I-23 | 353.9753 | 352.9816 | — | — | 38.531 | 1d | S29 |
| | diI-23 | 479.8719 | — | — | — | — | 1d | — |
| 24 | 24 | 290.0790 | 289.0838 | — | — | 31.263/31.372/31.712 | Std. | S8, S30 |
| | Cl-24 | 324.0401 | 323.0450 | 325.0415 | — | 33.137 | 1b | S30 |
| | diCl-24 | 358.0011 | — | — | — | — | 1b | — |
| | I-24 | 415.9757 | 414.9837 | — | — | 33.533/34.204 | 1d | S30 |
| | diI-24 | 541.8723 | — | — | — | — | 1d | — |

Note:
Although we looked for halogenation beyond two sites, we did not observe any for the substrates tested.
*All compounds were measured in negative mode except for 3,5-dimethoxyaniline (17) and fenoterol (23)

Crystal Structure of PltM and its Complex with Phloroglucinol

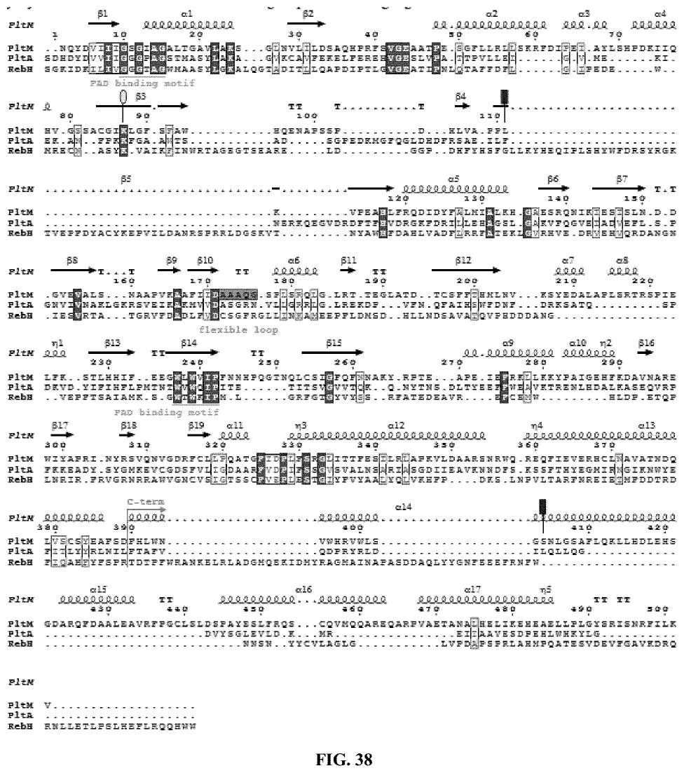

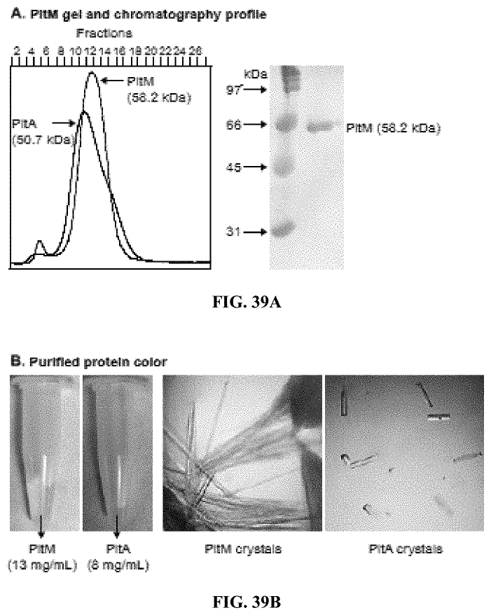

In addition to its remarkable halide versatility and a very broad substrate profile for a phenolic halogenase, PltM is at most ˜15% identical in sequence to other structurally characterized FAD-dependent halogenases, and it contains a unique C-terminal region (residues 390-502) (FIG. 38). Prompted by these intriguing properties, a 1.80 Å-resolution crystal structure of this enzyme was obtained (FIGS. 39A-B; Table 5). The crystal structure of PltM was obtained by the single anomalous dispersion (SAD) method by using ethylmercury derivatized crystals. PltM is a monomer in solution (FIGS. 39A-B); the crystals of PltM contain four nearly structurally identical monomers per asymmetric unit.

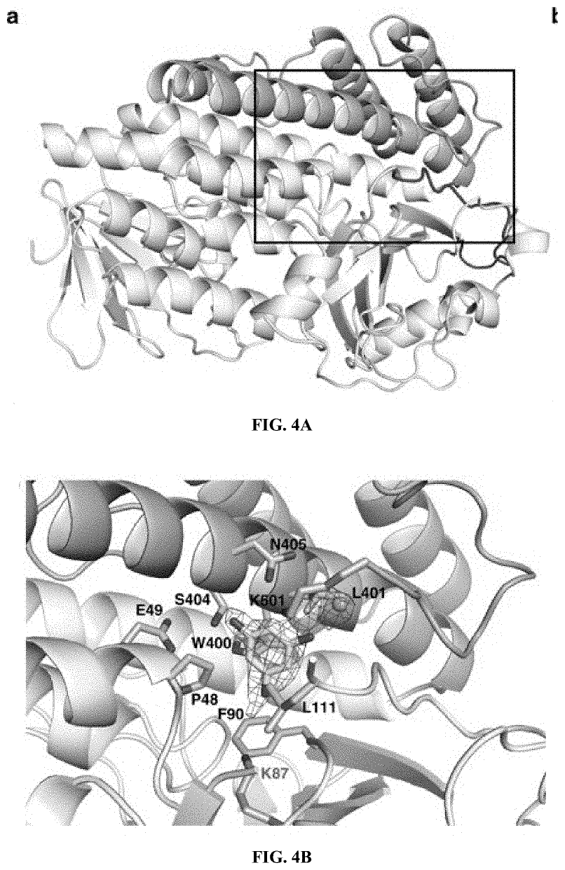

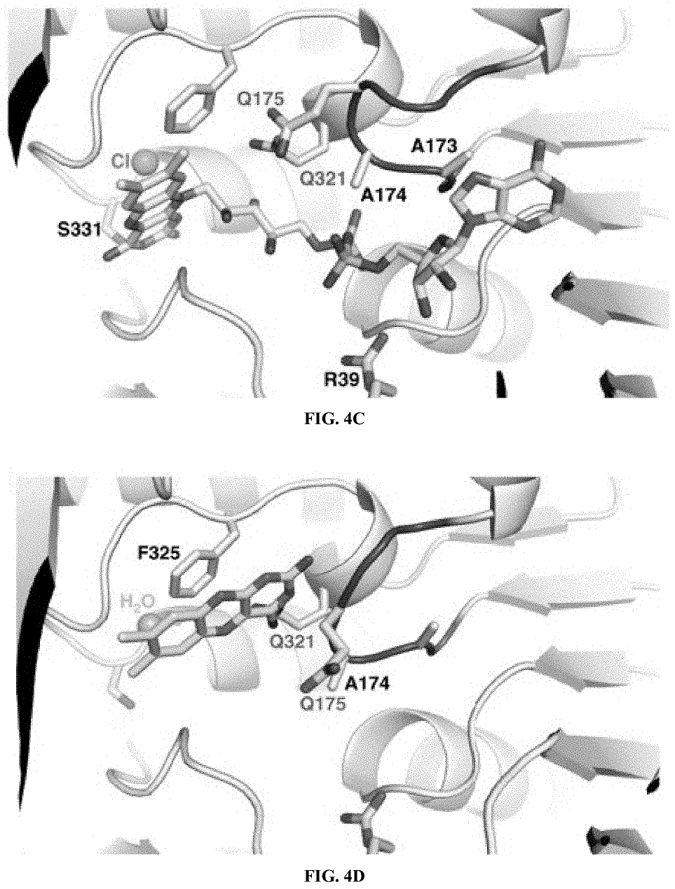

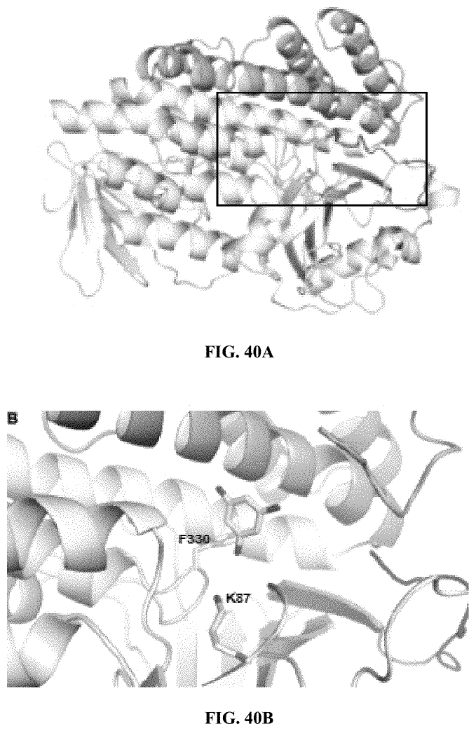

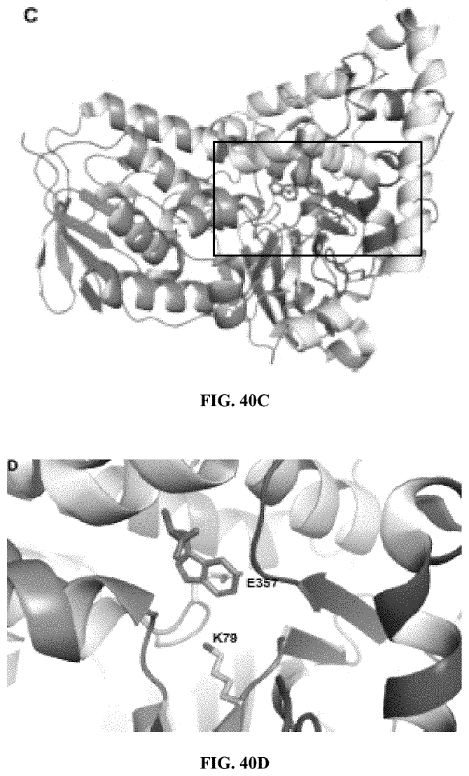

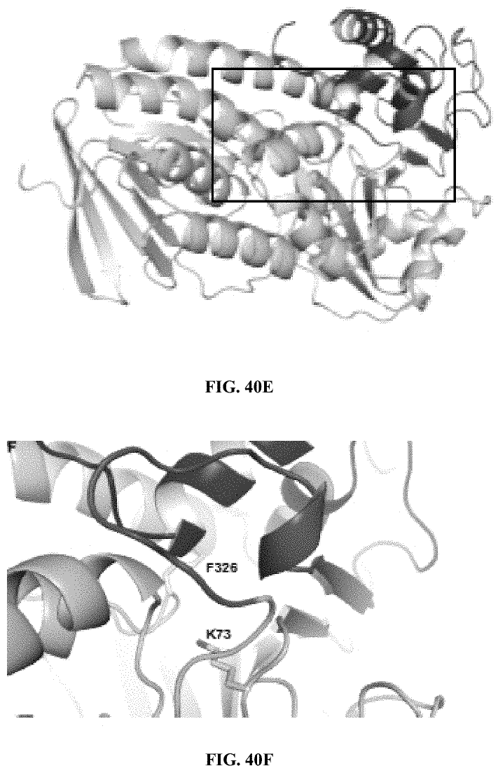

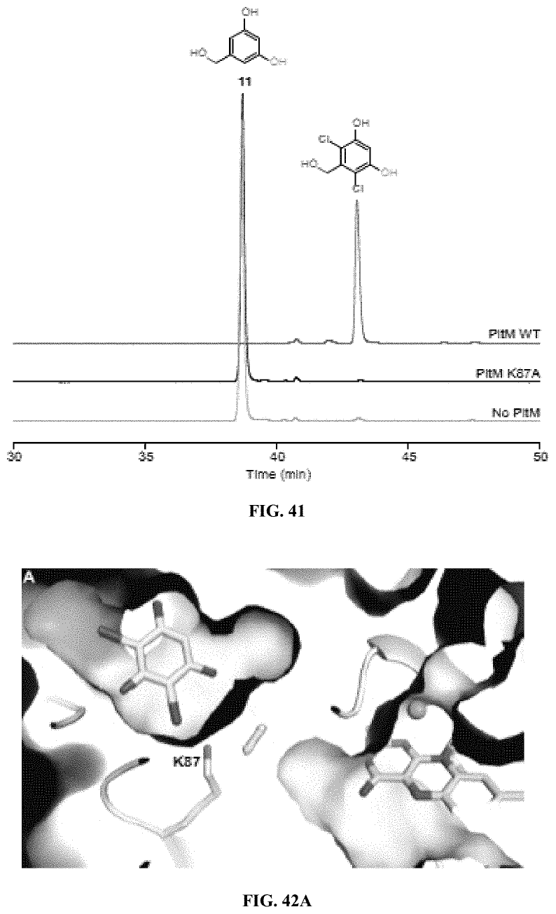

A monomer of PltM (FIG. 4A) consists of a large FAD binding fold that is conserved in FAD-dependent halogenases (residues 1-389). FAD and halide were not found in the FAD binding site, consistent with the lack of color of the protein and its crystals (FIG. 39B). The C-terminal quarter of the protein is a unique helical region not found in other halogenases (FIGS. 40A-F). The putative substrate binding cleft located in the interface of the FAD binding fold and the C-terminal region leads to a conserved catalytic lysine residue (Lys87), based on structural superimposition of PltM with structures of Trp halogenases bound to L-Trp (FIGS. 40A-F). Indeed, mutating Lys87 to an alanine yielded a catalytically inactive protein (FIG. 41). The C-terminal region then likely helps define the substrate specificity. Soaking crystals of PltM with compound 1 yielded a strong and featureful polder omit mFₒ-DF_{c} electron density in three out of four substrate binding sites in the asymmetric unit, corresponding to a molecule of compound 1 and a water molecule that bridged it with the protein (FIG. 4B).

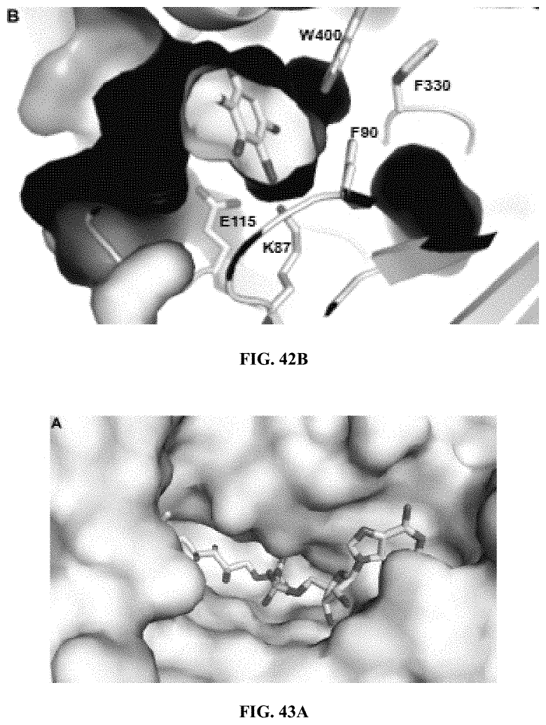

The binding site of compound 1 is analogous to that of L-Trp in the crystal structure of RebH and PrnA (FIGS. 40A-F). The nearest carbon atom of compound 1 that can be halogenated is ˜4.5 Å away from the Ne of Lys87, further supporting the model. At its entrance, the substrate binding cavity is lined by charged and polar side chains (Glu115, Glu49, Lys501, and Asn405) (FIG. 4B), which would interact favorably with hydroxyl and amino groups on PltM substrates indicated by the activity profile (FIG. 3B). One face of the phenyl ring of 1 is in nonpolar contacts with and Pro48 and Leu111 and the other face stacks approximately orthogonally Trp400 and interacts with Leu401. The phenyl ring of compound 1 is stacked nearly orthogonally against Phe90. This residue likely helps orient the substrate for halogenation. The hydroxyl groups of bound 1 are within hydrogen bonding distances from the side chains of Lys501, Asn405, Glu49, Ser404 and the main chain nitrogen of Phe90 and one hydroxyl is bridged to a carbonyl oxygen of Ile499 by a water molecule. These interactions underscore the importance of the unique C-terminal region in substrate recognition. The substrate binding site is large enough to accommodate a diiodinated 1 (FIGS. 42A-B). The substrate binding site is situated relatively close to the protein surface, which could allow access to larger substrates, like resveratrol (23). The halogenation center is nevertheless restricted by the helical C-terminal region to addition of up to two halogens; a trihalogenated product cannot be sterically accommodated and neither can halogenated L-Trp (FIGS. 40A-F).

TABLE 5
X-ray diffraction data collection and structure refinement
statistics for apo-PltM and apo-PltM-Hg.

| | PltM | | PltM-Hg | |
|---|---|---|---|---|
| PDB ID | 6BZN | | 6BZI | |
| Data collection | | | | |
| Space group | P2₁2₁2₁ | | P2₁2₁2₁ | |
| Number of monomers per asymmetric unit | 4 | | 4 | |
| Unit cell dimensions | | | | |
| a, b, c (Å) | 64.2, 157.1, 214.0 | | 63.7, 156.3, 216.1 | |
| α, β, γ (°) | 90, 90, 90 | | 90, 90, 90 | |
| Resolution (Å) | 39.88-1.80 | (1.83-1.80) [symbol] | 50.0-2.4 | (2.44-2.40) [symbol] |
| R_{merge} | 0.125 | (0.766) | 0.164 | (0.708) |
| I/σI | 14.5 | (1.7) [symbol] | 11.2 | (2.4) [symbol] |
| Completeness (%) | 99.5 | (96.3) | 96.3 | (92.2) |
| Redundancy | 6.7 | (6.1) | 6.7 | (6.5) |

| | | |
|---|---|---|
| Structure refinement statistics | | |
| Resolution (Å) | 39.88-1.80 | 45.0-2.4 |
| Number of unique reflections | 189997 | 78214 |
| R [symbol] /R_{free} | 0.161/0.189 | 0.209/0.256 |
| No. of atoms | | |
| Protein | 15852 | 15595 |
| Ligand/Ion | 67 | 71 |
| Water | 2015 | 451 |
| B-factors | | |
| Protein | 19.6 | 28.0 |
| Ligand/Ion | 31.4 | 27.3 |
| Water | 31.3 | 24.6 |
| R.m.s. deviations | | |
| Bond lengths (Å) | 0.02 | 0.008 |
| Bond angles (°) | 1.71 | 1.19 |
| Ramachandran plot statistics^{b} | | |
| % of residues in favored region | 98.1 | 98.3 |
| % of residues in allowed region | 1.9 | 1.7 |
| % of residues in outlier region | 0 | 0 |
| Ligands/Ions | Glycerol (10) [symbol] | Glycerol (3) |
| | Calcium (7) | Calcium (4) |
| | | Mercury (25) |
| | | Ethylmercury (8) |

^{a}Numbers in parentheses indicate the values in the highest-resolution shell.
^{b}Indicates Rampage statistics. [symbol]
^{c}Number of ligands in the asymmetric unit. [symbol]
indicates data missing or illegible when filed

Crystal Structures of PltM with FAD Bound in Different States

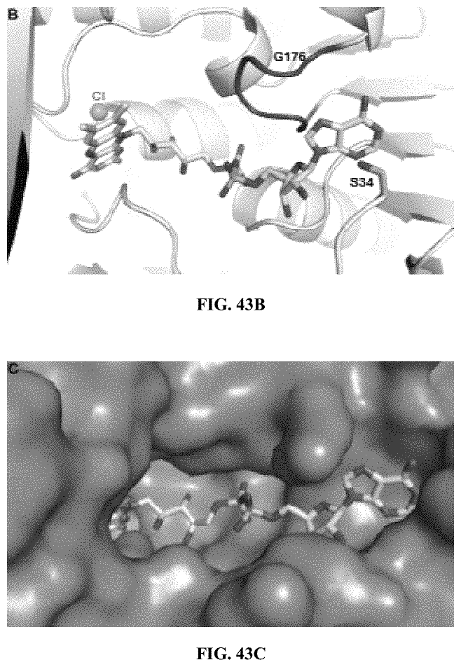

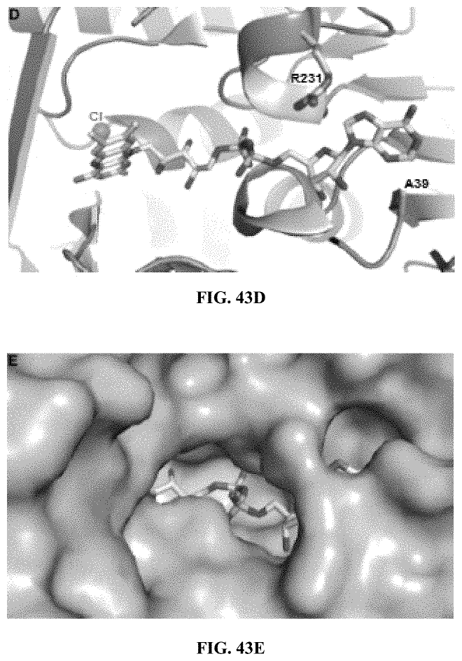

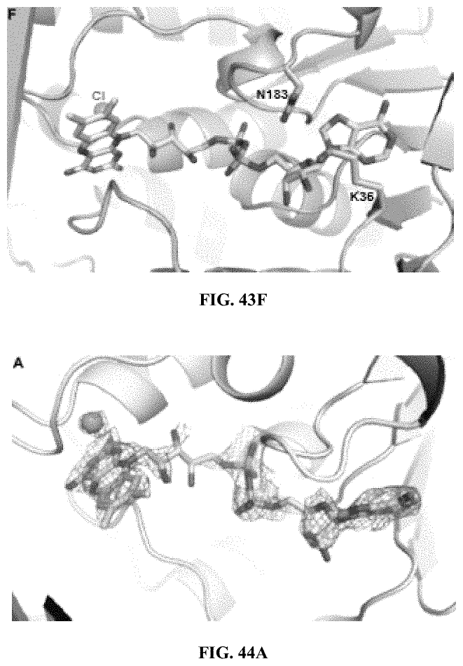

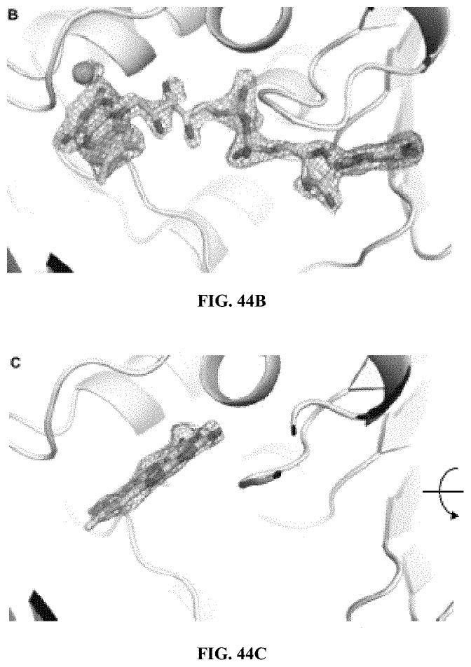

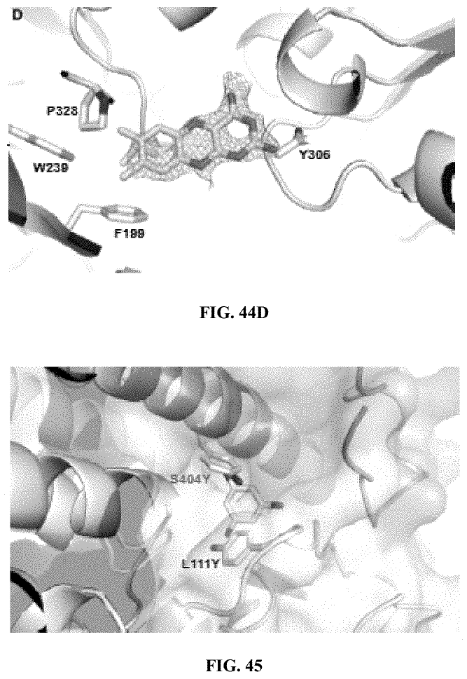

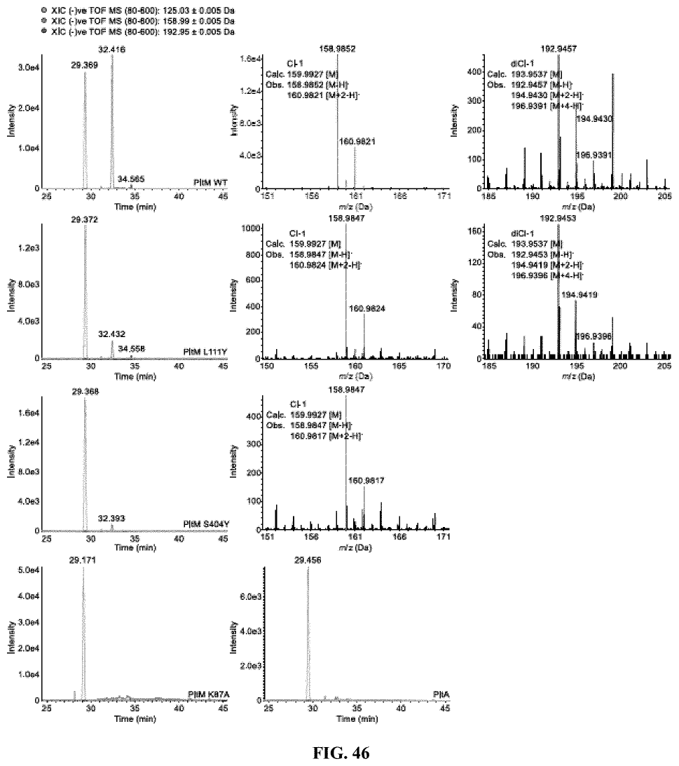

PltM represents a type of FAD-dependent enzyme, where FAD dissociates out of its binding site for reduction. To gain structural insight into this enigmatic process, a crystal structure of PltM-FAD complex was determined by soaking the crystals of apo PltM with FAD. Two different crystal forms of PltM-FAD complexes were obtained, where a molecule of FAD was bound to PltM in two different states (FIGS. 4C-D; Table 5). In one state, an FAD molecule was bound at a site and orientation analogous to those observed in structures of other FAD-dependent halogenases, where the isoalloxazine group of the FAD was fully encased by the enzyme (FIGS. 4C and 43A-F). A chloride ion was well resolved at a conserved site near the FAD. In the other state, the FAD molecule was bound near the mouth of the FAD binding cleft, with the clearly resolved isoalloxazine ring in the same plane, but oriented perpendicularly to the fully bound state, also making extensive contacts with the protein (FIG. 4D). The electron density for rest of the FAD molecule is not observed due to disorder (FIGS. 44A-D), as in this state the adenine nucleotide moiety is directed into the solvent. This structure may represent an intermediate between the apo and the fully bound FAD state. The crystals of PltM-FAD complexes in this state belong to the same crystal form as the crystals of all other complexes in this study; therefore, crystal packing interactions have no effect on the FAD binding state.

A short nonconserved loop containing three Ala, a Gly and a Ser (residues 172-178) and the side chain of Gln321 are in two different conformations in these two structures (FIGS. 4C-D). In the holoenzyme state, the loop and Gln321 form one side of the narrow cleft holding the adenine nucleotide portion of FAD in place: the side chain of Ala173 interacts with the adenine ring of the FAD, Ala174 interacts with the phosphosugar bridge, and the aliphatic portion of Gln321 holds the riboflavin bridge. In the state with partially bound FAD, this cleft is collapsed, and filled with water. In this state, the isoalloxazine ring is sandwiched between Phe325 and the backbone of loop residues Ala174 and Gln175, including the Cb of the latter residue. The FAD binding pocket does not contain a Cl⁻, indicating that a halide ion binds upon the final steps of FAD binding. Previous kinetic experiments with RebH and p-hydroxybenzoate hydroxylase suggested that kinetically significant conformational changes involving FAD dynamics occurred in FAD recycling. For both enzymes, it was proposed that a distinct mechanistically important state exists where the flavin ring of FAD can undergo redox chemistry, while being sufficiently shielded away from the solvent. This structure may represent such intermediate.

Halogenation Assays in Fermentation Culture

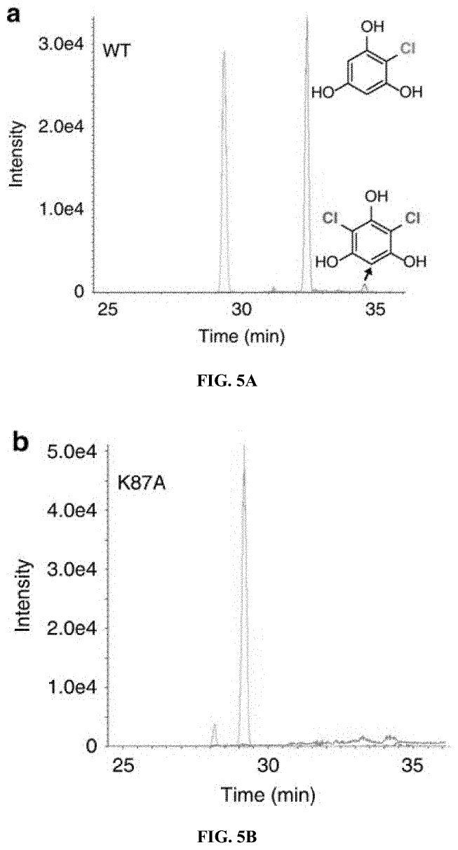

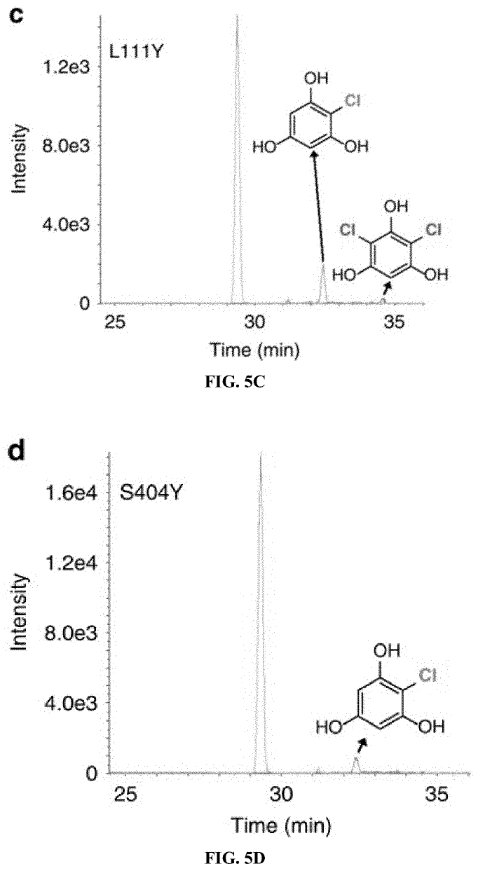

As a preliminary assessment of potential use of PltM in a fermentation setting, the ability to halogenate phloroglucinol (1) upon addition to the culture of E. coli BL21(DE3) overexpressing PltM was tested. The substrate binding cavity observed in the crystal structures was also validated by testing halogenation by two PltM point mutants of PltM, L111Y and S404Y, in this setting. These two residues (one from the FAD binding fold and one from the C-terminal region) line the substrate binding cavity, and their bulkier substitutions are predicted to block binding of 1 (FIG. 45). In addition, as negative controls, PltM K87A that was demonstrated to be inactive in vitro as well as PltA were used. All five proteins were expressed at the same level. The cells expressing wild-type PltM generated mono- and dichlorinated 1 (FIGS. 5A-D and 46). No halogenated product was observed in cultures expressing PltM K87A and PltA, validating the PltM as the sole source of halogenation activity. For the cells expressing L111Y and S404Y mutants, the product yield was significantly reduced compared to wild-type; the effect of the S404Y mutation was especially severe.

A crystal structure of PltM L111Y was determined, which showed that the overall protein structure is unperturbed and the only effect of the mutation was to obstruct the access to the substrate binding pocket, as predicted (FIG. 45). S404Y caused a more drastic effect than L111Y because Y404 was predicted to sterically clash with the bound substrate. These data further validated the structure-based definitions of the substrate binding site and suggested a potential for halogenation in a fermentation setting.

Kinetics and Regiospecificity of PltM in Optimized Reactions

For quantitative analysis of enzyme kinetics and detailed structural characterization of reaction products, as well as for potential future biotechnological use, in vitro enzymatic reaction conditions were extensively optimized and enzymes were coupled to maximize product yield. The critical factors of the optimized conditions were introducing glucose dehydrogenase (GDH) for NADPH regeneration and lowering the concentrations of NADPH and halide salts. This optimization significantly improved reaction yields, resulting in full conversion of several substrates (Table 8). This additional information corroborated the preference for substrates containing electron-withdrawing groups and showed preference of PltM for substrates with 1- and 3-hydroxyl or amino groups.

TABLE 8
Overall yield of optimized chlorination reactions
for different substrates of PltM.

| | % overall conversion^{a} |
|---|---|
| Substrate | (trial 1, 2)^{b} |
| 2 | 57, 25 |
| 3 | 100, 100 |
| 6 | 1, 4 |
| 8 | 100, 100 |
| 9 | 97, 96 |
| 10 | 3, 2 |
| 11 | 100, 100 |
| 12 | 28, 26 |
| 13 | 5, 3 |
| 15 | 34, 7 |
| 16 | 100, 100 |
| 18 | 100, 100 |
| 23 | 24, 20 |

^{a}% overall conversion is the sum of all chlorinated products.
^{b}Yields of two independent reactions are reported.

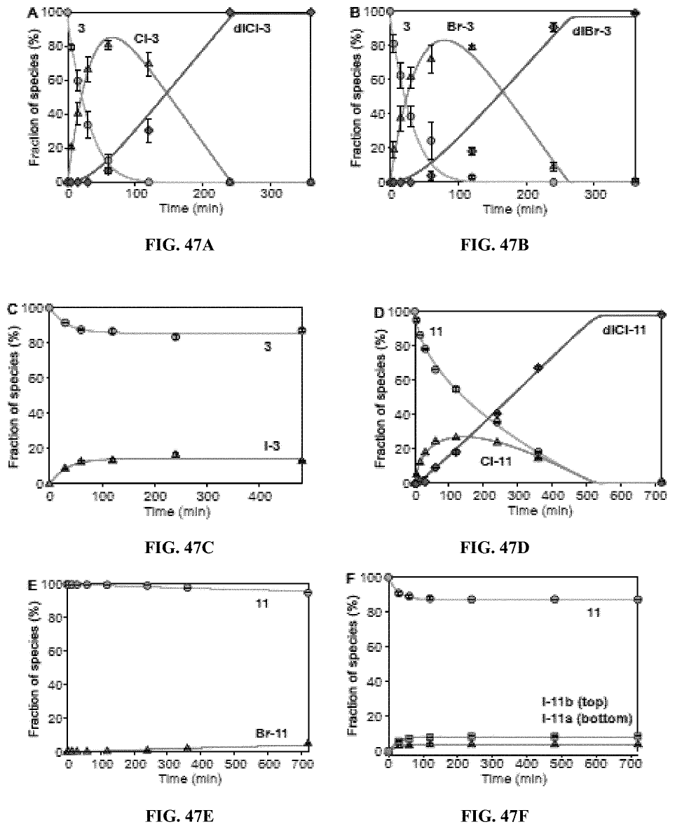

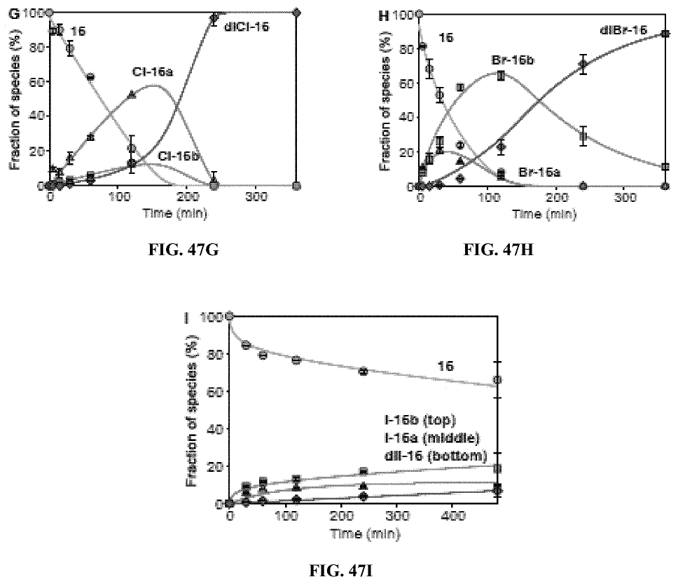

The halide preference was determined and the kinetics of chlorination and bromination of substrates 3, 11, and 16 was evaluated quantitatively, which showed 100% conversion upon overnight reaction (FIGS. 47A-I; Table 9). Kinetic of iodination could not be analyzed quantitatively due to gradual enzyme precipitation in the presence of iodide. These data indicated that PltM preferred chlorination for all substrates that were eventually dichlorinated. The preference for bromination versus iodination depended on particular substrates, with 3 and 16 showing preference for bromination, and 11 for iodination. In fact, both 3 and 16 were dibrominated by PltM. Chlorination and bromination of 3 and 16 occurred with similar efficiencies, whereas 11 was chlorinated much better than brominated or iodinated. Interestingly, two mono-halogenated products were observed for iodination of 11 and for halogenation of 16. No fluorination was still observed for any substrates at the optimized conditions.

TABLE 9
Kinetic parameters for halogenations of selected substrates.

| Substrate | Halogen | Substrate/Product | k_{cat} (min⁻¹) | Kₘ (μM) | k_{cat}/Kₘ (min⁻¹μM⁻¹) |
|---|---|---|---|---|---|
| 3 | Cl | 3/Cl-3 | 2.3 ± 0.1^{a} | (7.6 ± 1.3) × 10⁻² | 30 ± 5 |
| | | Cl-3/diCl-3 | 0.40 ± 0.01 | 0.11 ± 0.02 | 3.5 ± 0.6 |
| | Br | 3/Br-3 | 1.9 ± 0.1 | 0.71 ± 0.2 | 2.7 ± 0.6 |
| | | Br-3/diBr-3 | 0.38 ± 0.01 | 0.78 ± 0.17 | 0.49 ± 0.11 |
| 11 | Cl | 11/Cl-11 | 1.6 ± 0.1 | 8.4 ± 0.4 | 0.19 ± 0.01 |
| | | Cl-11/diCl-11 | 0.18 ± 0.01 | 0.44 ± 0.02 | 0.41 ± 0.02 |
| | Br | 11/Br-11 | 0.24 ± 0.02 | (1.1 ± 0.2) × 10³ | (2.2 ± 0.2) × 10⁻⁴ |
| 16 | Cl | 16/Cl-16a^{b} | 0.35 ± 0.01 | 1.1 ± 0.3 | 0.31 ± 0.09 |
| | | 16/Cl-16b | 0.22 ± 0.01 | 1.1 ± 0.3 | 0.19 ± 0.06 |
| | | Cl-16a/diCl-16 | 0.95 ± 0.01 | 18 ± 5 | (5.2 ± 1.4) × 10⁻² |
| | | Cl-16b/diCl-16 | 1.0 ± 0.01 | 15 ± 9 | (7.0 ± 4.4) × 10⁻² |
| | Br | 16/Br-16a | 0.47 ± 0.04 | 151 ± 20 | (3.1 ± 0.5) × 10⁻³ |
| | | 16/Br-16b | 2.2 ± 0.1 | 151 ± 20 | (1.5 ± 0.2) × 10⁻² |
| | | Br-16a/diBr-16 | 0.21 ± 0.02 | 6.0 ± 1.2 | (3.5 ± 0.8) × 10⁻² |
| | | Br-16b/diBr-16 | 2.0 ± 0.1 | (1.3 ± 0.1) × 10³ | (1.5 ± 0.2) × 10⁻³ |

^{a}The values of all mono-halogenation and dihalogenation rate constants k_{cat, 1} and k_{cat, 2} respectively, and Kₘ for mono-halogenation and dihalogenation (defined as (k_{d, 1} + k_{cat, 1})/k_{a, 1} and (k_{d, 2} + k_{cat, 2})/k_{a, 2}, respectively) were determined by nonlinear regression using Dynafit, as described in Methods.
^{b}Two distinct mono-halogenation products of the same reaction are denoted by labels a and b.

The high yield of chlorination and bromination of these and several other compounds allowed the present inventors to establish the regiospecificity of the halogenation by PltM. However, some substrates or products were insufficiently stable during halogenation reactions precluding their quantitative structural analysis. The structures of the final dichlorinated products of 3, 8, 9, 11, 15, 16, 18, as well as the monochlorinated product of 23 and the dibrominated product of 3 were determined by NMR spectroscopy. The resulting products were 4,6-diCl-3, 4,6-diCl-8, 2,4-diCl-9, 2,6-diCl-11, 3,5-diCl-15, 4,6-diCl-16, 4,6-diCl-18, 4-Cl-23, and 4,6-diBr-3, respectively (FIGS. 6 and 49-66). These structures were consistent with the time course experiments showing one mono-halogenated intermediate for symmetrical substrates 3 and 11 and two mono-halogenated intermediates for asymmetrical substrate 16. Likewise, for most other substrates (8, 15, and 18) the structures of the respective monochlorinated intermediates are unambiguously inferred owing to the product symmetry.

These results show that for mono- or di-hydroxylated or aminated substrates, PltM halogenates almost exclusively in ortho to these polar groups, but not between them. However, when a methyl or a styrene moiety was found in meta to two hydroxyls, as in compound 9 (which was dichlorinated) and resveratrol (23; which was monochlorinated), respectively, a chlorination event occurred between the two hydroxyls.

Development of an Immobilized Halogenating System

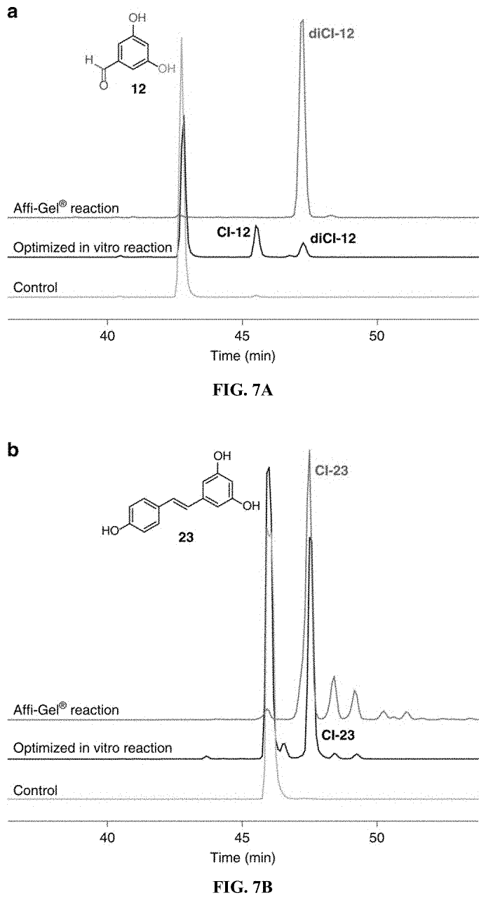

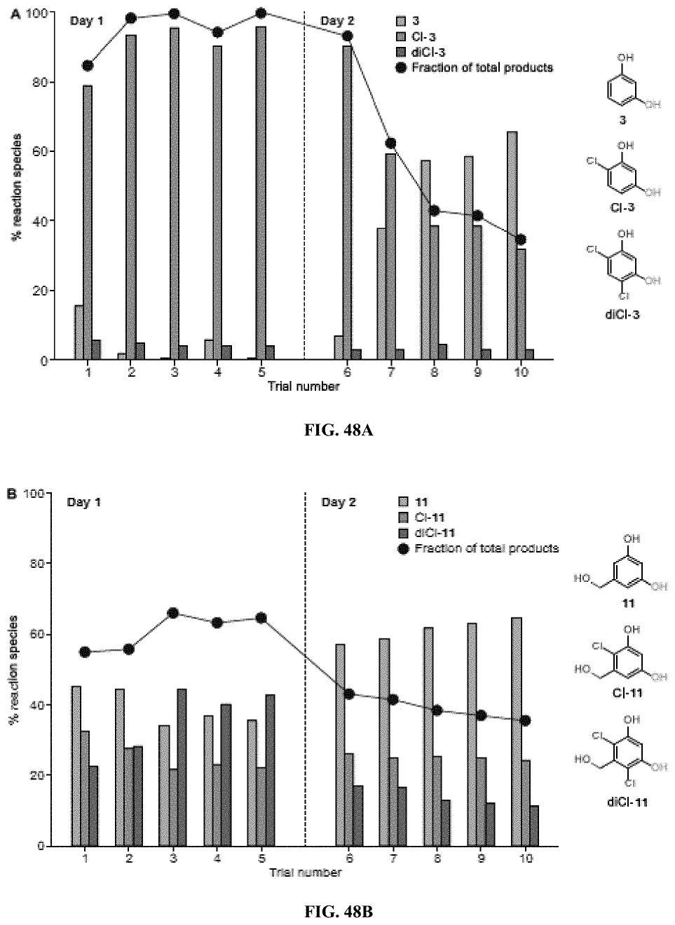

The halogenation yield is limited by stability of proteins, with PltM being the limiting factor. To achieve a more efficient and scalable halogenation reaction, the present inventors developed a method where all three proteins were immobilized on agarose resin (Affi-Gel® 15), packed into a spin column, and then used as a resin conjugate for halogenation. The halogenation reactions were performed by adding substrate and reagents into the column. This protein bound resin showed a high halogenation yield for some compounds, which could not be efficiently halogenated by free enzymes in solution (FIGS. 7A-B). Notably, the enzyme-resin conjugate could be reused 5-6 times without significant loss of efficiency (FIGS. 48A-B).

The remarkable halide versatility for any FAD-dependent halogenase and very broad substrate profile for a phenolic halogenase call for future exploration of PltM as a halogenation tool. The structures discussed herein revealed a unique architecture of this enzyme, and an FAD orientation that may be relevant to the FAD recycling mechanism shared by FAD binding enzymes.

Methods

Materials and Instrumentation

The PltM, SsuE, and PltA (used as a control in this study) proteins were overexpressed and purified based on our previously described protocols. DNA primers for PCR were purchased from Integrated DNA Technologies (IDT; Coralville, Iowa, USA). Restriction enzymes, Phusion DNA polymerase, and T4 DNA ligase were purchased from New England BioLabs (NEB; Ipswich, Mass., USA). All chemicals and buffer components were purchased from Sigma-Aldrich or VWR (Radnor, Pa., USA) and used without any further purification. Size-exclusion chromatography was performed on a fast protein liquid chromatography (FPLC) system BioLogic DuoFlow (Bio-Rad; Hercules, Calif., USA) by using a HiPrep 26/60 S-200 HR column (GE Healthcare, Piscataway, N.J., USA). Liquid chromatography-mass spectrometry (LC-MS) was performed on a Shimadzu high-performance liquid chromatography (HPLC) system equipped with a DGU-20A/3R degasser, LC-20AD binary pumps, a CBM-20A controller, a SIL-20A/HT autosampler (Shimadzu, Kyoto, Japan), and Vydac HPLC DENALI™ Column (C18, 250×4.6 mm, 5∝cm particle size) from Grace (Columbia, Md., USA) and an AB SCIEX TripleTOF 5600 (AB SCIEX, Redwood City, Calif.) mass spectrometer recording in negative or positive mode between 80 and 600 m/z. HPLC was performed on an Agilent Technologies 1260 Infinity system equipped with a Vydac HPLC DENALI™ column (C18, 250×4.6 mm, 5∝cm particle size) and an Alltech Econosil HPLC column (C18, 250×10 mm, 10∝cm particle size; Grace) for analytical and semi-preparative experiments, respectively. ¹H and ¹³C NMR spectra were recorded at 400 and 500 (for ¹H) as well as 100 MHz (for ¹³C) on a Varian 400 MHz spectrometer, using deuterated solvents as specified. Chemical shifts (d) are given in parts per million (ppm). Coupling constants (J) are given in Hertz (Hz), and conventional abbreviations used for signal shape are as follows: s, singlet; d, doublet; t, triplet; m, multiplet; dd, doublet of doublets; ddd, doublet of doublet of doublets; br s, broad singlet; dt, doublet of triplets.

Synthesis of Compound 15

Aluminum chloride (1.3 g, 9.99 mmol) was slowly added to a solution of phloroglucinol (1, 315 mg, 2.50 mmol) in 1:1/1,2-dichloroethane:nitrobenzene (10 mL) at 0° C. After stirring this mixture at this temperature for 10 min under a nitrogen atmosphere, acetyl chloride (0.21 mL, 3.00 mmol) was added. Then the ice bath was removed, and the mixture stirred at 80° C. for 2 h. The reaction progress was monitored by TLC (1:2/EtOAc:Hexanes, R_{f} 0.35). The reaction mixture was quenched with H₂O (60 mL), extracted with EtOAc (2×100 mL), washed with brine (20 mL), and then dried over MgSO₄. The organic layer was removed under reduced pressure and the residue was purified by flash column chromatography (SiO₂, 1:2/EtOAc:Hexanes) to afford the known compound 15³⁰ (223 mg, 53%) as a yellow solid: ¹H NMR (400 MHz, CD₃OD) δ 5.78 (s, 2H), 2.58 (s, 3H); ¹³C NMR (100 MHz, (CD₃)₂SO) δ 203.1, 164.9, 164.5, 104.2, 94.1, 31.3.

PltM Mutagenesis

PltM mutants K87A, L111Y, and S404Y were constructed by splicing-by-overlap-extension method. The sequences downstream and upstream of the mutation site were amplified first individually from ppltM-pET28a(NHis). For PltM K87A mutant the primer pairs were: 5′-CGCCTGCGGGATCgcgCTGGGCTTCAGTTTTG-3′ (SEQ ID NO: 1) with 5′-CATACTCGAGCTAGACTTTGAGGATGAAACGATTG-3′(SEQ ID NO: 2); and 5′-CAAAACTGAAGCCCAGcgcGATCCCGCAGGCG-3′ (SEQ ID NO: 3) with 5′-GCAGCTCTCATATGAATCAGTACGACGTCATTATC-3′ (SEQ ID NO: 4). For PltM L111Y mutant the primers were: 5′-CTTGTGGCCCCGCCGtatAAGGTGCCGGAAGCC-3′ (SEQ ID NO: 5) with SEQ ID NO: 2; and 5′-GGCTTCCGGCACCTTataCGGCGGGGCCACAAG-3′ (SEQ ID NO: 6) with SEQ ID NO: 4. For PltM S404Y mutant, the primer pairs were: 5′-CTGGCTCAGCGGCtatAACCTGGGCAGTGC-3′ (SEQ ID NO: 7) with SEQ ID NO: 2; and 5′-GCACTGCCCAGGTTataGCCGCTGAGCCAG-3′ (SEQ ID NO: 8) with SEQ ID NO: 4. The PCR products of the above primer pairs were used as templates for another round of PCR using primers SEQ ID NO: 2 and SEQ ID NO: 4. The products from the second round of PCR were digested with restriction enzymes NdeI and XhoI and ligated into NdeI/XhoI-linearized pET28a, yielding ppltMK87A-pET28a, ppltML111Y-pET28a, and ppltMS404Y-pET28a. The mutations were verified by DNA sequencing (Eurofins Genomics).

Preparation of Pgdh-pET28a Overexpression Construct

The glucose dehydrogenase (gdh) gene was amplified from genomic DNA of Bacillus subtilis subsp. subtilis 168 by PCR with the forward and reverse primers: 5′-AGGATGCATATGTATCCGGATTTAAAAGGAAAAG-3′ (SEQ ID NO: 9) and 5′-CGCTTTCTCGAGTTAACCGCGGCCTGCCTGGAAT-3′ (SEQ ID NO: 10), respectively. The PCR product was purified by agarose gel extraction and digested by restriction enzymes NdeI and XhoI, which was subsequently ligated into NdeI/XhoI-linearized pET28a. The resulting plasmid pgdh-pET28a was transformed into a chemically competent E. coli TOP10 strain, and the cloning was verified by sequencing of the purified plasmids.

Preparation of PltM and Coupled Enzymes for In Vitro Assays

Open reading frames encoding PltM and FAD reductase SsuE were cloned into E. coli expression vectors as previously reported. For production of PltM, SsuE, and GDH, the expression vectors were transformed into E. coli BL21 (DE3) (ATCC; Manassas, Va.). In each case, a colony was grown overnight at 37° C. with shaking at 200 rpm in LB medium (5 mL) supplemented with 50 μg/mL kanamycin. These overnight cultures were inoculated into LB medium (1 L) supplemented with 50 μg/mL kanamycin. Cultures were grown (37° C., 200 rpm) until an attenuance at 600 nm of 0.6 was reached. At this time, protein expression was induced by adding isopropyl-β-D-1-thiogalactopyranoside (IPTG, 0.2 mM), and the cultures were incubated at 16° C. with shaking at 200 rpm for an additional 20 h. The cells were harvested by centrifugation at 3,000×g for 10 min at 4° C. The cell pellets were washed with buffer A (50 mM sodium phosphate pH 7.4, 400 mM NaCl, 5 mM imidazole, and 10% glycerol). The cells were resuspended in 40 mL of buffer A supplemented with 1 mM dithiothreitol (DTT) and 1 mM phenylmethanesulfonyl fluoride (PMSF). The cells were then lysed by intermittent sonication, followed by clarification by centrifugation at 40,000×g for 45 min at 4° C. The supernatants were incubated with 0.5 mL of pre-washed Ni^{II}-NTA agarose resin (Qiagen, Valencia, Calif.) at 4° C. for 2 h with slow tumbling. The slurry was loaded onto a column and washed with 2×5 mL of buffer A followed by elution with a gradient of imidazole concentration in buffer A (2×5 mL of 20 mM, 5 mL of 40 mM, 5 mL of 60 mM, 2×5 mL of 250 mM). Fractions containing pure proteins were combined and dialyzed against 3×2 L of buffer B (50 mM sodium phosphate pH 7.4, 2 mM β-mercaptoethanol (βME), and 10% glycerol). Each of the three dialysis steps was performed at least for 4 h. The dialyzed proteins were concentrated to ˜20 mg/mL for PltM and GDH or ˜2.5 mg/mL for SsuE by using Amicon Ultra-15 Centrifugal Filter Units (EMD Millipore, Billerica, Mass., USA) with 10-kDa molecular weight cutoff (MWCO) for PltM and GDH or 3-kDa MWCO for SsuE, and protein concentrations were determined by absorbance at 280 nm with calculated extinction coefficients ε=59,840 M⁻¹cm⁻¹, ε=20,340 M⁻¹cm⁻¹, and ε=29,910 M⁻¹cm⁻¹ for PltM, SsuE, and GDH, respectively (protcalc.sourceforge.net). The total yields of pure PltM, SsuE, and GDH were 17.6 mg, 6.0 mg, and 10.3 mg from 1 L of culture, respectively. The proteins were flash frozen in liquid nitrogen and stored at −80° C. for biochemical assays. The point mutants of PltM were purified by using the above protocol for the full-length PltM.

Preparation of PltM for Crystallography

Wild-type PltM and PltM L111Y mutant were purified as described above with an additional size-exclusion chromatography step. Wild-type PltM and PltM L111Y eluted from Ni^{II} resin were loaded onto an S-200 column equilibrated in 40 mM Tris-HCl pH 8.0, 100 mM NaCl, 2 mM βME. Fractions containing NHis₆-PltM were pooled and concentrated to 40 mg/mL by using an Amicon Ultra-15 Centrifugal Filter Unit with 10 kDa MWCO. Purified PltM proteins were kept on ice for crystallization studies.

In Vitro Assays of PltM with Various Substrates and Halides

The halogenation assays were carried out similarly to a recently described procedure. The substrates that have been tested are given in FIGS. 3A and 14. For substrate profile determination (Assay 1), 100 μL reactions were carried out in 30 mM sodium phosphate pH 7.4. As a halide source, 200 mM of either NaF (Assay 1a), NaCl (Assay 1b), NaBr (Assay 1c), or NaI (Assay 1d) was used. To ensure that PltM is incapable of fluorinating, an additional 200 μL reaction with 300 mM NaF was run. A 200 μL reaction with 400 mM NaBr was also run to ensure no additional bromination reaction occurred. Each reaction also contained a specified substrate (0.5 mM), FAD (0.2 mM), NADPH (5 mM), PltM (5.5 μM), and SsuE (5.0 μM). The reactions were initiated by adding NADPH under N₂. The reaction tubes were tightly closed to avoid contact with air. The reaction mixtures were incubated at 25° C. for 3 h prior to extraction with EtOAc (4×100 μL). The organic layer was dried by a gentle flow of air, and the residue was dissolved in MeOH to prepare 1-10 μg/mL samples for LC-MS analysis.

To establish if hetero-dihalogenation by PltM could be observed, halogenating competition assays in 1:1 or 10:1 mixtures of two different halide salts were performed (Assay 2). The reactions contained the same components as above except single halide salts were replaced with either a 1:1/NaCl:NaBr (Assay 2a), 1:1/NaCl:NaI (Assay 2b), or 1:1/NaBr:NaI (Assay 2c) mixtures (100 mM of each halide). The reactions were initiated by adding NADPH under N₂. A 1:1/NaCl:NaBr reaction was also performed with 200 mM of each halide to test occurrence of homo-di- or hetero-chlorination/bromination, and 10:1/NaCl:NaI (Assay 2d) and 10:1/NaBr:NaI (Assay 2e) mixtures with 200 mM of NaCl or NaBr and 20 mM of NaI were tested to check whether chlorination or bromination could occur in the presence of iodide and whether iodination can occur with chlorination or bromination to yield a C1,I-substrate or Br,I-substrate. The reactions were incubated and processed as described above in Assay 1.

Optimized In Vitro PltM Halogenation Assay

To increase production of halogenated molecules and decrease the amount of NADPH required, the above in vitro assay was optimized by using an additional enzyme, glucose dehydrogenase (GDH). The optimized reaction mixture contained substrate (0.5 mM for chlorination and bromination; 0.25 mM for iodination; prepared from 50 mM stock in DMSO), FAD (5 μM), NADPH (5 μM), PltM (6 μM), SsuE (5 μM), GDH (0.5 μM), glucose (20 mM), NaX (10 mM for chlorination and bromination; 0.5 mM for iodination), and sodium phosphate (30 mM, pH 7.4), and was incubated at room temperature. The overall yield of halogenation products was determined for reactions run overnight for several substrates (Table 8). Conversion of the substrate to halogenated products was monitored by HPLC at λ=275-320 nm, where the absorbance of molecules is not affected by halogenation, and quantified as fraction of reaction species (%).

The time course experiments for kinetic analysis were performed in 100 μL reaction mixtures by quenching the reactions at 0, 5, 15, 30, 60, 120, 240, and 360 min (for 3 and 16), and an additional 720 min (for 11) for chlorination and bromination, and at 0, 30, 60, 120, 240, and 480 min (for 3 and 16), or an additional 720 min (for 11) for iodination. The time course experiments were performed in duplicate. Compound 1 was unstable under these optimized conditions, and it was not tested. The in vitro analysis of K87A mutant was performed overnight in 100 μL reaction mixture by using the compound 11 as a substrate. Wild-type PltM was used as a positive control, and no enzyme reaction was used as a negative control. In all above reactions, the compounds were extracted with EtOAc (4×100 μL) and dried under gentle air flow. The products were dissolved in MeOH (30 μL for chlorination and bromination; 15 μL for iodination) for HPLC analysis.

The scale-up experiments were performed overnight in 25 mL for compound 23, in 50 mL for compounds 3, 8, 9, 11, 15, and 18, or 100 mL for 16. PltM concentration was 25 μM with compounds 15 and 250 μM with compound 23. To process the chlorination reaction of compound 23, ice-cold MeOH (50 mL) was added to precipitate the proteins. This mixture was incubated for 2 h at −20° C., and the protein precipitate was removed by centrifugation (40,000×g, 30 min, 4° C.). The pellet was washed by ice-cold MeOH (50 mL) and centrifuged down again (40,000×g, 15 min, 4° C.). The supernatant was combined in a round bottomed flask, and MeOH was removed by in vacuo. The products were extracted with EtOAc (4× reaction volume) and dried in vacuo. These were dissolved in MeOH (0.5-1 mL) for purification by semi-preparative HPLC.

Halogenation Assay Using Immobilized Enzymes

To increase the yield of halogenation reaction and make the enzymes reusable, PltM, SsuE, and GDH, we immobilized these proteins on Affi-Gel® 15 resin (Bio-Rad, Hercules, Calif.). To increase the stability of the coupled enzymes, GDH from Bacillus amyloliquefaciens SB5 (GDH-BA) was used in this assay. This enzyme was expressed and purified, as described above, from a pET23a vector (amp^{R}) containing a synthetic gene encoding this enzyme (NCBI accession # JQ305165) with an NHis₆ tag, purchased from GenScript (Piscataway, N.J.). The enzymes were dialyzed into buffer C, which contains HEPES (50 mM, pH 7.5), βME (2 mM), and glycerol (10%). Suspended Affi-Gel® resin (250 pL) was transferred into a QIAquick spin column (Qiagen), and the resin was washed three times with 500 μL of H₂O and buffer D (30 mM HEPES, pH 7.5). For each time, the wash solution was removed by centrifugation (400×g, for 15-30 s, 4° C.). The washed resin was incubated with SsuE (˜50 μM, 300 μL) for 4 h at 4° C. The beads were washed with buffer D twice and subsequently incubated with a mixture of GDH-BA (˜200 μM, 50 μL) and PltM (˜500 μM, 250 μL) overnight at 4° C. This resin-enzyme conjugate was washed twice with buffer D and preserved in 4° C. in buffer D until needed. For each 250 μL resin, 300 μL of reaction solution, which contained substrate (0.5 mM), FAD (5 μM), NADPH (5 μM), glucose (20 mM), NaCl (10 mM), and HEPES (30 mM, pH 7.5), was used. The reaction with resveratrol (23) was performed overnight at room temperature. The reaction solution was collected by centrifugation (400×g, every 15-30 s until the solution was removed, 4° C.), and the resin-enzyme conjugate in the column was washed with buffer D (300 μL) three times. These solutions were extracted with EtOAc (4×300 μL) and dried in vacuo. The solid material was dissolved in MeOH (200 μL) and analyzed by HPLC (FIGS. 7A-B). The reusability of the resin-enzyme conjugate was tested with substrates 3 and 11 (FIGS. 48A-B). The reactions (same as above) were run for 1 h at room temperature and processed as described above. After processing the reaction, the same reaction was repeated four more times. After the 5^{th} reaction, the beads were stored at 4° C. overnight in buffer D. The 6^{th}-10^{th} reactions were performed in the following day.

Kinetic Analysis of PltM Halogenation

To determine the halogenation preference, the kinetic parameters were obtained by the global nonlinear regression analysis of all reaction species using DynaFit software for the following halogenation mechanism:

$$\begin{matrix}E + S {⇄}_{{k}_{d , 1}}^{{k}_{a , 1}} E \cdot S & ( 1 ) \\ E \cdot S {\to }^{{k}_{cat , 1}} E \cdot {P}_{1} & ( 2 ) \\ {EP}_{1} + S {⇄}_{{k}_{d , 2}}^{{k}_{a , 2}} {EP}_{1} \cdot S & ( 3 ) \\ {EP}_{1} \cdot S {\to }^{{k}_{cat , 2}} E \cdot {P}_{2} & ( 4 )\end{matrix}$$

where E, S, P₁, P₂ are enzyme, substrate, mono- and dihalogenated product, respectively.

Cell-Based Activity Assay of PltM

E. coli BL21 (DE3) cells were transformed with ppltM-pET28a, ppltMK87A-pET28a, ppltML111Y-pET28a, ppltMS404Y-pET28a, and ppltA-pET28a. The ppltA-pET28a plasmid overexpressing the halogenase PltA whose substrate is pyrrolyl-S-PltL (a peptidyl carrier protein-linked pyrrole) was used as a negative control. Five colonies from each transformant were cultured in 2×500 mL of LB medium (for ppltM-pET28a, ppltML111Y-pET28a, and ppltMS404Y-pET28a) and 1×500 mL of LB medium (for ppltMK87A-pET28a and ppltA-pET28a) with 50 μg/mL kanamycin at 37° C. and 200 rpm until attenuance of 0.2 at 600 nm. The cultures were then moved to 25° C. until attenuance of 0.5. Protein expression was induced by adding 0.2 mM IPTG to all seven flasks, and the cultures were incubated with shaking for 1 h. 12.5 μg/mL of compound 1 was added to 1×500 mL of LB medium containing ppltM-pET28a, ppltMK87A-pET28a, ppltML111Y-pET28a, ppltMS404Y-pET28a, and ppltA-pET28a. Compound 1 was not added to the three remaining flasks (negative controls). After additional incubation for 20 h, the cells were pelleted at 5,000 g for 10 min, and the supernatant was collected. The supernatant was extracted with EtOAc (3×330 mL), which was dried in vacuo. This was then dissolved in MeOH (100 μL) prior to addition of H₂O (800 μL) followed by centrifugation at 20,000×g for 10 min to remove the precipitate. The supernatant was collected and 1 μL was diluted into 199 μL of MeOH for LC-MS analysis (Table 7).

TABLE 7
LC/MS data for cell-based assays.

| | | | Obs. | Obs. | Obs. | | | |
|---|---|---|---|---|---|---|---|---|
| | | Calcd. | mass | mass | mass | Retention | | |
| | | mass | [M − H]⁻ | [M + 2 − H]⁻ | [M + 4 − H]⁻ | time | Assay | Fig. |
| Enzyme | Product | (Da) | (Da) | (Da) | (Da) | (min) | # | # |
| PltM WT | I | 126.0317 | 125.0243 | — | — | 29.369 | Std. | 3, S39 |
| | Cl-1 | 159.9927 | 158.9852 | 160.9821 | — | 32.416 | 3 | 3, S39 |
| | diCl-1 | 193.9537 | 192.9457 | 194.9430 | 196.9391 | 34.565 | 3 | 3, S39 |
| PltM K87A | I | 126.0317 | 125.0249 | — | — | 29.171 | Std. | 3, S39 |
| | Cl-1 | 159.9927 | — | — | — | — | 3 | — |
| | diCl-1 | 193.9537 | — | — | — | — | 3 | — |
| PltM L111Y | I | 126.0317 | 125.0246 | — | — | 29.372 | Std. | 3, S39 |
| | Cl-1 | 159.9927 | 158.9847 | 160.9824 | — | 32.432 | 3 | 3, S39 |
| | diCl-1 | 193.9537 | 192.9453 | 194.9419 | 196.9396 | 34.558 | 3 | 3, S39 |
| PltM S404Y | I | 126.0317 | 125.0245 | — | | 29.368 | Std. | 3, S39 |
| | Cl-1 | 159.9927 | 158.9847 | 160.9817 | — | 32.393 | 3 | 3, S39 |
| | diCl-1 | 193.9537 | — | — | — | — | 3 | — |
| PltA | I | 126.0317 | 125.0244 | — | — | 29.456 | Std. | S39 |
| | Cl-1 | 159.9927 | — | — | — | | 3 | — |
| | diCl-1 | 193.9537 | — | — | — | | 3 | — |

Note:
Although we looked for trihalogenated 1, we did not observe any. All masses were measured in negative mode.

HPLC and LC-MS Analysis of Halogenated Products

The halogenation reaction products were analyzed by HPLC or LC-MS by injecting 10 μL of each sample. The compounds were separated by Reversed-phase HPLC at the flow rate of 0.2 mL/min by using the following program: eluent A=H₂O; eluent B=MeCN; gradient=2% B for 5 min, increase to 100% B over a 30 min period, stay at 100% B for 9 min, decrease to 2% B over a 1 min period, and re-equilibrate the column at 2% B for 30 min.

For HPLC analysis, the molecules were observed by absorbance at λ=275 nm as described above. As necessary, the following mass spectrometer was operated in negative and positive modes with the following parameters: For negative mode, mass range, 80-600 m/z in profile mode; temperature, 550° C. and ion spray voltage floating, −4500 V, and for positive mode, mass range, 80-600 m/z in profile mode; temperature, 550° C. and ion spray voltage floating, 4500 V. The presence of each compound was analyzed by extracted ion chromatograph (XIC) with the expected mass ±0.05 Da for Assay 1 and Assay 2 and ±0.005 Da for Assay 3 (FIGS. 2A-C, 8-13, and 17-37; Tables 3-4 and 7).

The LC-MS was operated by Analyt TF Software (SCIEX, Framingham, Mass.), and the data was analyzed by PeakView (SCIEX). To purify 4 selected scaled-up halogenated products, semi-preparative HPLC was performed by injecting 100 μL per injection at 1 mL/min by using the following gradient program with eluent A as H₂O (with 0.1% TFA) (for compounds 3 and 11) or 10 mM ammonium bicarbonate (for 16) and eluent B as MeCN: 2% B for 10 min, increase to 100% B over a 40 min period, stay at 100% B for 5 min, decrease to 2% B over a 1 min period, followed by re-equilibration in 2% B for 9 min. The collected peak fractions were dried under reduced pressure and lyophilized for NMR analysis.

NMR analysis of products of large-scale halogenation

The exact position for the various halogenations were determined either by comparison with commercially available standards (4,6-dichlororesorcinol) or by a combination of HMBC and HSQC experiments.

The analysis of halogenation products is presented as follows:

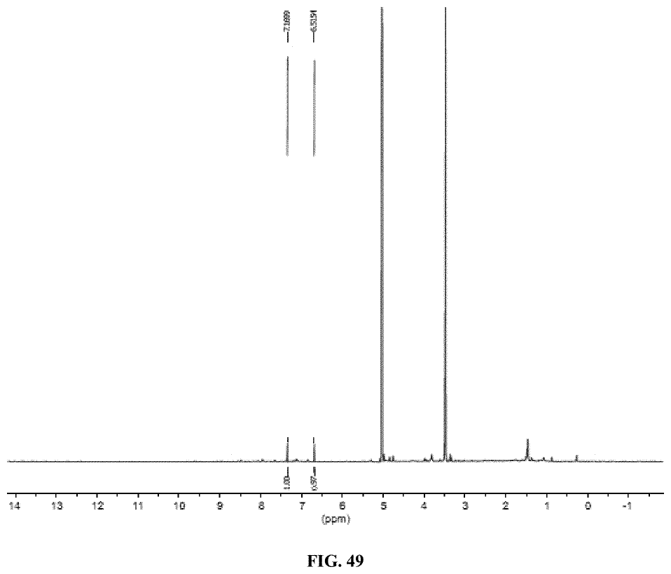

Analysis of 4,6-dichlororesorcinol (4,6-diCl-3): ¹H NMR (500 MHz, CD₃OD, FIG. 49) δ 7.17 (s, 1H), 6.52 (s, 1H).

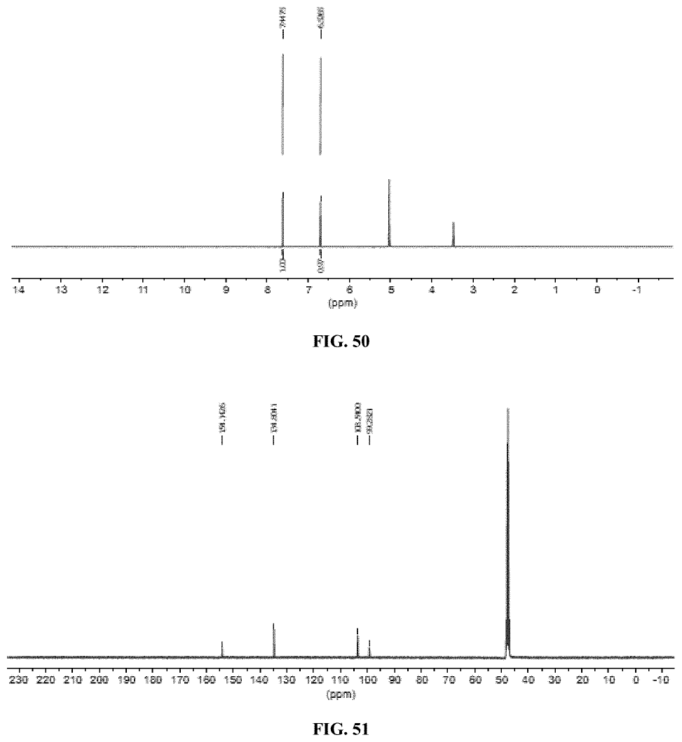

Analysis of 4,6-dibromoresorcinol (4,6-diBr-3): ¹H NMR (500 MHz, CD₃OD, FIG. 50) δ 7.45 (s, 1H), 6.53 (s, 1H); ¹³C NMR (100 MHz, CD₃OD, FIG. 51) δ 154.1, 134.8, 103.5, 99.3.

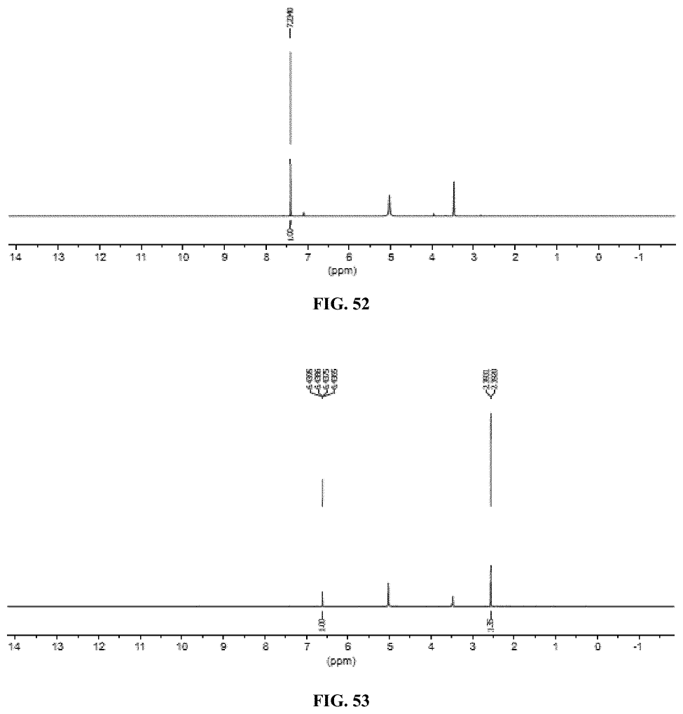

Analysis of 2,4,6-trichlororesorcinol (4,6-diCl-8): ¹H NMR (500 MHz, CD₃OD, FIG. 52) δ 7.23 (s, 1H).

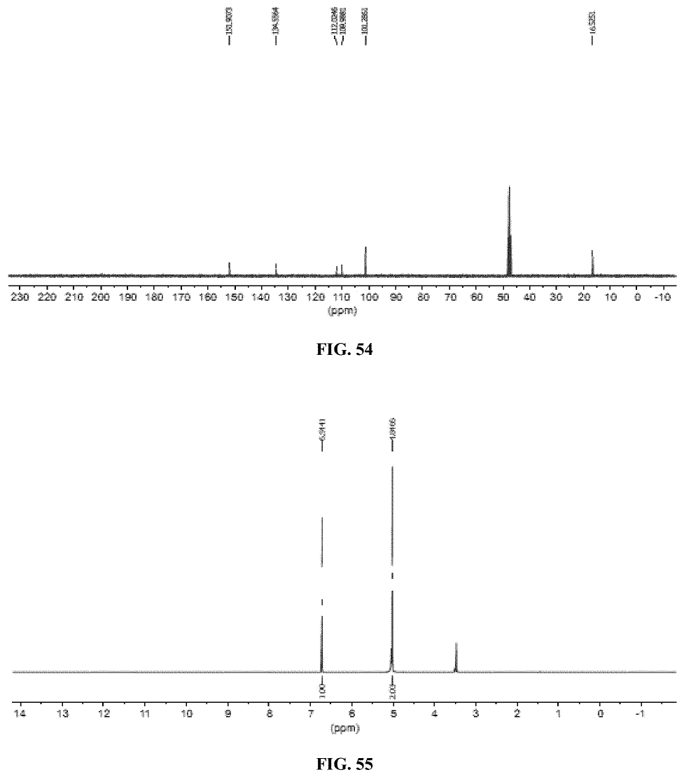

Analysis of 2,4-dichloro-5-methylresorcinol (2,4-diCl-9): ¹H NMR (500 MHz, CD₃OD, FIG. 53) δ 6.43 (q, J=0.5 Hz, 1H), 2.39 (d, J=0.5 Hz, 3H); ¹³C NMR (100 MHz, CD₃OD, FIG. 54) δ 151.9, 134.6, 112.0, 110.0, 101.3, 16.5.

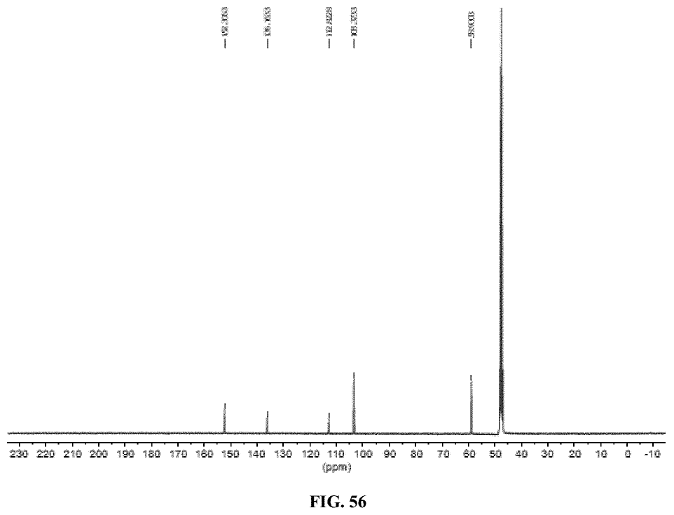

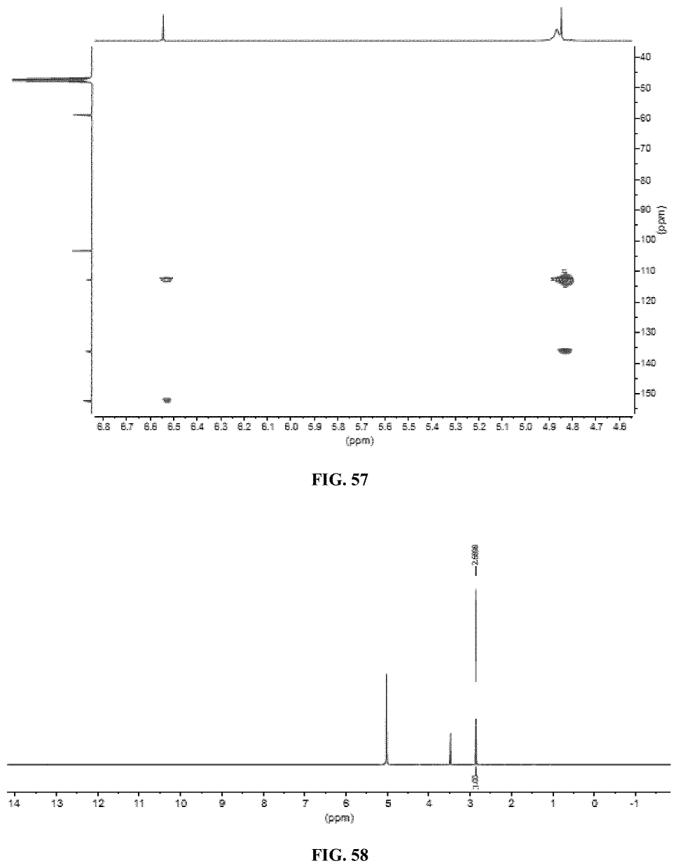

Analysis of 2,6-dichloro-3,5-dihydroxybenzyl alcohol (2,6-diCl-11): ¹H NMR (500 MHz, CD₃OD, FIG. 55) δ 6.55 (s, 1H), 4.85 (s, 2H); ¹³C NMR (100 MHz, CD₃OD, FIG. 56) δ 152.3, 136.2, 112.8, 103.4, 58.9. The HMBC for 2,6-dichloro-3,5-dihydroxybenzyl alcohol is presented in FIG. 57.

Analysis of 3,5-dichloro-2,4,6-trihydroxyacetophenone (3,5-diCl-15): ¹H NMR (500 MHz, CD₃OD, FIG. 58) δ 2.69 (s, 3H).

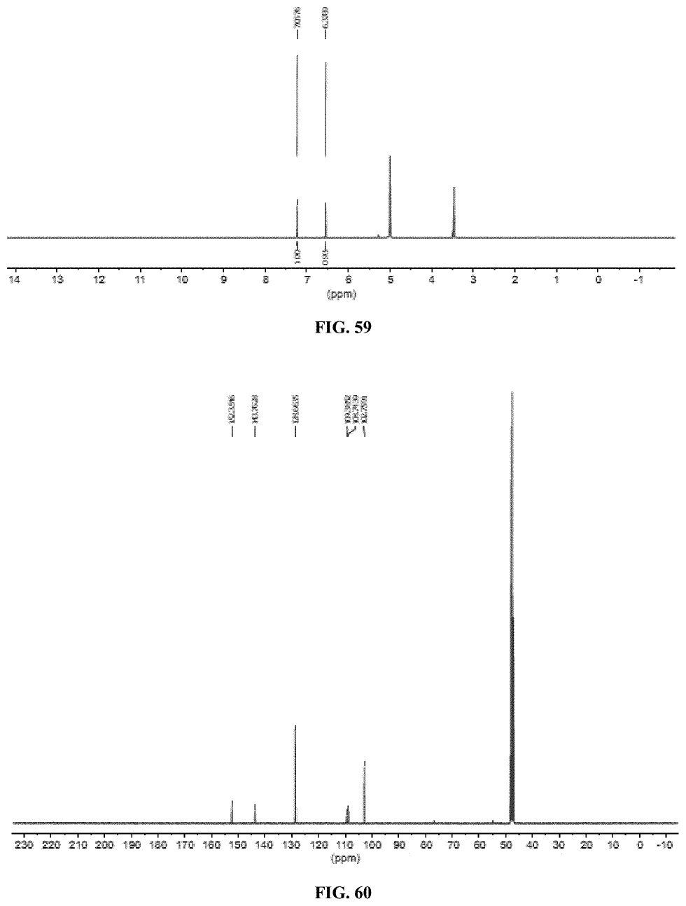

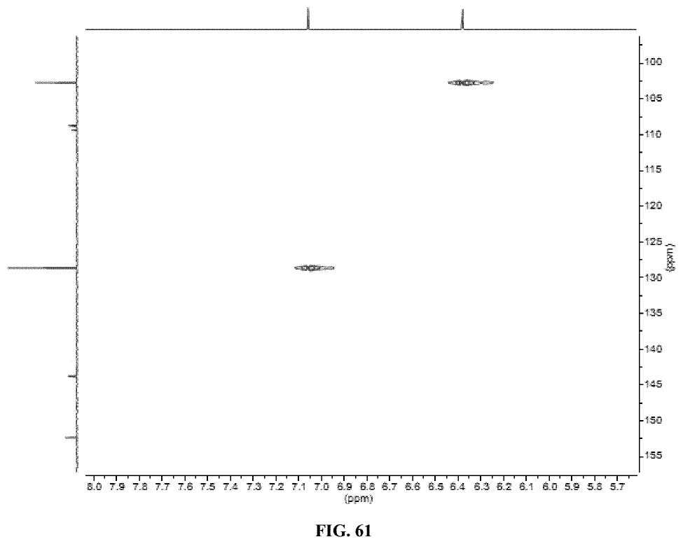

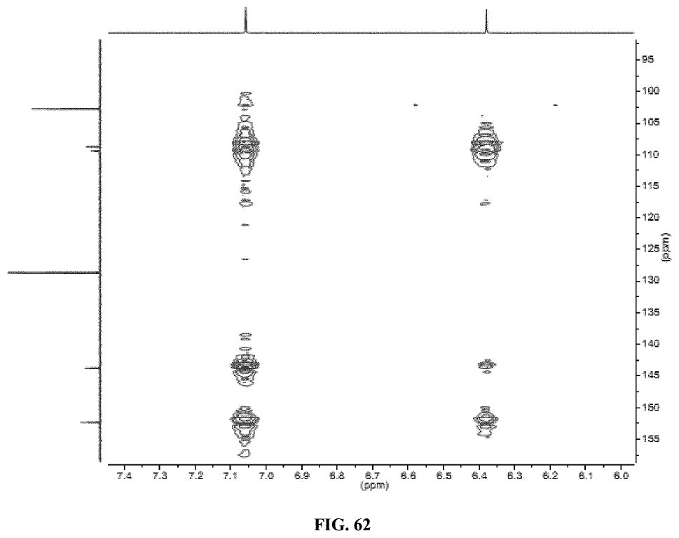

Analysis of 5-amino-2,4-dichlorophenol (2,4-diCl-16): ¹H NMR (400 MHz, CD₃OD, FIG. 59) δ 7.06 (s, 1H), 6.38 (s, 1H); ¹³C NMR (100 MHz, CD₃OD, FIG. 60) δ 152.4, 143.8, 128.7, 109.4, 108.7, 102.8. The HSQC and HMBC for 5-amino-2,4-dichlorophenol are presented in FIGS. 61 and 62, respectively.

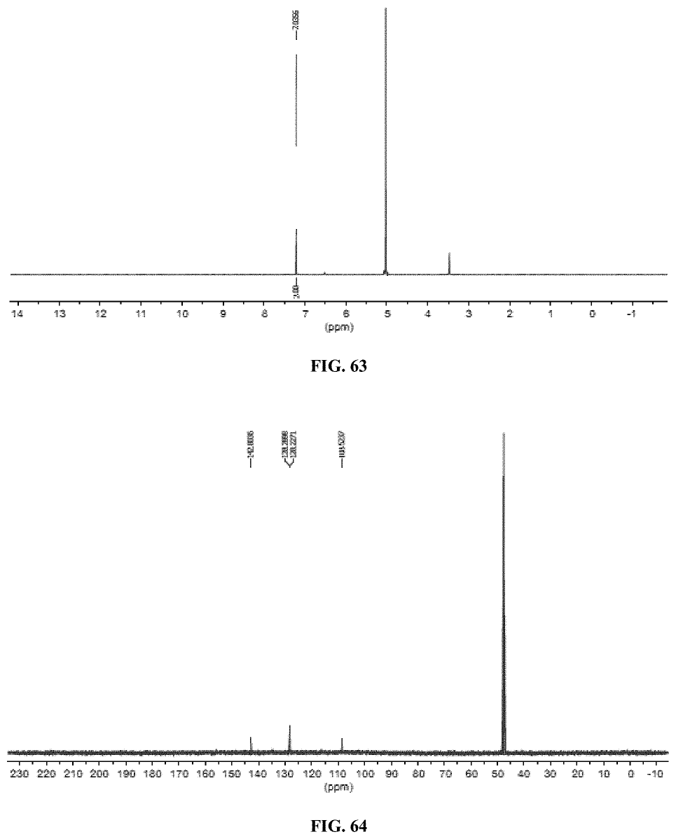

Analysis of 2,4-dichloro-1,5-diaminobenzene (2,4-diCl-18): ¹H NMR (500 MHz, CD₃OD, FIG. 63) δ 7.04 (s, 2H); ¹³C NMR (100 MHz, CD₃OD, FIG. 64) δ 142.8, 128.3, 128.2, 108.5.

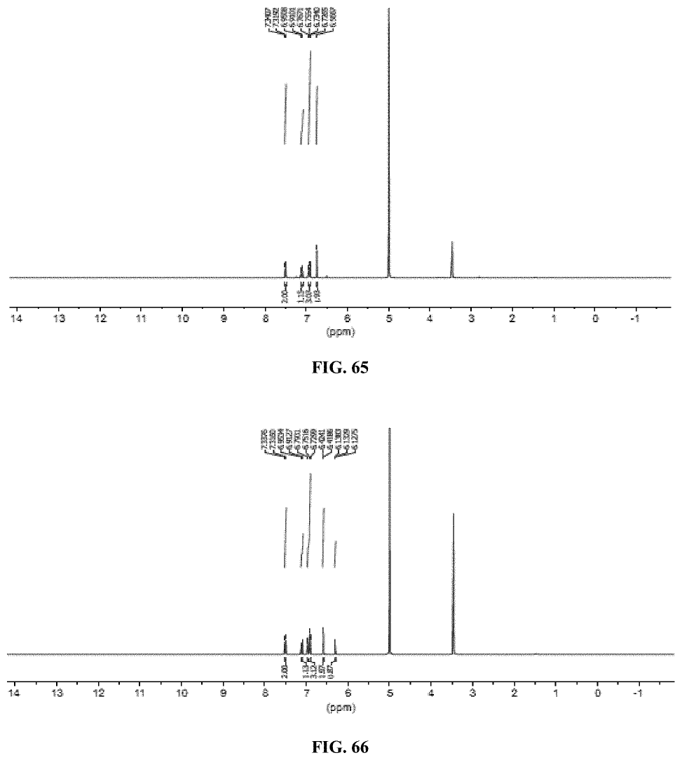

Analysis of 4-chloro-resveratrol (4-Cl-23): ¹H NMR (400 MHz, CD₃OD, FIG. 65) δ 7.33 (d, J=8.6 Hz, 2H), 6.93 (d, J=16.2 Hz, 1H), 6.75 (d, J=16.2 Hz, 1H), 6.74 (d, J=8.6 Hz, 2H), 6.57 (br s, 2H).

Analysis of resveratrol (23): ¹H NMR (400 MHz, CD₃OD, FIG. 66) δ 7.33 (d, J=8.6 Hz, 2H), 6.93 (d, J=16.2 Hz, 1H), 6.77 (d, J=16.6 Hz, 1H), 6.74 (d, J=8.6 Hz, 2H), 6.42 (d, J=2.2 Hz, 2H), 6.13 (t, J=2.2 Hz, 1H).

Crystallization of PltM

PltM crystals were obtained by the hanging drop method with drops containing 0.5 μL of PltM (40 mg/mL) and 0.5 μL of the reservoir solution (0.1 M Tris pH 8, 0.2 M NaCl, 0.1 M CaCl₂) and 12-17% PEG 8000). The drops were equilibrated against 0.5 mL of reservoir solution at 21° C. Long rod-shaped crystals appeared after 1-3 days. The crystals were cryoprotected by a gradual transfer to the solution with the same composition as the reservoir solution, additionally containing 20% glycerol. The crystals were then frozen by a rapid immersion into liquid nitrogen.

Determination of the Crystal Structure of PltM

PltM does not contain a sufficient number of Met residues for structure determination by using anomalous signal from selenium atoms in Se-Met PltM. However, PltM contains eight Cys residues, which, if accessible, would react with Hg salts. Hg derivative crystals of PltM were prepared by transferring native crystals from its mother liquor to the reservoir solution containing 1 mM ethyl mercury phosphate (EMP) and incubated overnight. These crystals were cryoprotected similarly to the native crystals. X-ray diffraction data for this and other crystals of PltM were collected at 100 K at the wavelength of 1 Å at synchrotron beamline 22-ID at the Advanced Photon Source at the Argonne National Laboratory (Argonne, Ill.). All datasets were indexed, integrated and scaled using HKL2000. The structure was determined by the single anomalous dispersion (SAD) method from the EMP derivative data set (using the wavelength of 1.0 Å), as follows. A heavy atom search by using direct method-based SHELXD program initially yielded a substructure of 22 Hg atoms in the asymmetric unit. This Hg substructure was used as an input in Autosolve in PHENIX suite to obtain initial phases, which were bootstrapped by difference Fourier analysis to yield the total of 33 Hg atoms and a readily interpretable electron density map, with the figure of merit of 0.71 after density modification. The structure of the Hg-derivatized PltM was then iteratively built by using COOT and refined by using REFMAC5 (Table 5).

The refined structure contained four monomers of PltM and 33 Hg atoms coordinated to Cys residues per asymmetric unit. A monomer of PltM from this structure was then used as a search model to determine the structure of native PltM by molecular replacement with Phaser in CCP4i suite. The native crystal structure of PltM was then iteratively adjusted and refined by using COOT and REFMAC5, respectively. Table 5 contains data collection and structure refinement statistics for this and other crystal structures in this study. The crystal structure coordinates and structure factor amplitudes for all crystal structures were deposited in the Protein Data Bank under accession codes specified in Tables 5 and 6.

TABLE 6
X-ray diffraction data collection and structure refinement statistics for PltM-FAD-
phloroglucinol, PltM-FAD, PltM L111Y-FAD and PltM-FAD intermediate complexes.

| | PltM-FAD- | | | | PltM | | PltM-FAD | |
|---|---|---|---|---|---|---|---|---|
| | phloroglucinol | | PltM-FAD | | L111Y-FAD | | partially bound | |
| PDB ID | 6BZA | | 6BZQ | | 6BZT | | 6BZZ | |
| Data collection | | | | | | | | |
| Space group | P2₁2₁2₁ | | P2₁2₁2₁ | | P2₁2₁2₁ | | P2₁2₁2₁ | |
| Number of monomers per | 4 | | 4 | | 4 | | 4 | |
| asymmetric unit | | | | | | | | |
| Unit cell dimensions | | | | | | | | |
| a, b, c (Å) | 64.2, 157.0, 213.7 | | 63.3, 157.7, 213.5 | | 64.0, 157.5, 213.0 | | 63.82, 157.2, 214.0 | |
| α, β, γ (°) | 90, 90, 90 | | 90, 90, 90 | | 90, 90, 90 | | 90, 90, 90 | |
| Resolution (Å) | 49.71-2.60 | (2.64-2.60) | 35.00-2.75 | (2.81-2.75) [symbol] | 50.00-2.10 | (2.14-2.10) [symbol] | 49.55-2.05 | (2.09-2.05) [symbol] |
| R_{merge} | 0.172 | (0.663) | 0.15 | (0.82) | 0.197 | (0.989) | 0.175 | (0.805) |
| I/σI | 14.0 | (2.2) [symbol] | 12.0 | (2.0) [symbol] | 10.7 | (1.9) [symbol] | 18.1 | (3.0) [symbol] |
| Completeness (%) | 98.9 | (99.5) | 96.0 | (97.5) | 94.7 | (92.6) | 98.2 | (93.9) |
| Redundancy | 6.0 | (6.0) | 4.6 | (4.6) | 5.4 | (4.3) | 7.5 | (7.1) |

Structure refinement statistics

| | | | | |
|---|---|---|---|---|
| Resolution (Å) | 40.00-2.60 | 35.00-2.75 | 35.0-2.10 | 40.00-2.05 |
| Number of unique reflections | 62405 | 54483 | 113768 | 125848 |
| [symbol] / [symbol] | 0.203/0.253 | 0.230/0.261 | 0.207/0.244 | 0.219/0.245 |
| No. of atoms | | | | |
| Protein | 15796 | 15796 | 15899 | 15801 |
| Ligand/Ion | 79 | 218 | 228 | 76 |
| Water | 104 | 164 | 943 | 455 |
| B-factors | | | | |
| Protein | 47.1 | 35.4 | 24.9 | 21.7 |
| Ligand/Ion | 70.7 | 67.3 | 36.0 | 33.6 |
| Water | 35.5 | 25.6 | 27.7 | 20.5 |
| R.m.s. deviations | | | | |
| Bond lengths (Å) | 0.007 | 0.007 | 0.007 | 0.007 |
| Bond angles (°) | 1.17 | 1.095 | 1.219 | 1.207 |
| Ramachandran plot statistics [symbol] | | | | |
| % of residues in favored region | 97.8 | 98.0 | 98.0 | 98.4 |
| % of residues in allowed region | 2.2 | 2.0 | 2.0 | 1.6 |
| % of residues in outlier region | 0 | 0 | 0 | 0 |
| Ligand/Ions | phloroglucinol (3) | FAD (4) | FAD (4) | FAD (4) |
| | FAD (2) | Chloride (4) | Chloride (5) | Calcium (4) |
| | Chloride (2) | Bromide (2) | Bromide (9) | |
| | | | Calcium (2) | |

^{a}Numbers in parentheses indicate the values in the highest-resolution shell.
^{b}Indicates Rampage statistics. [symbol]
^{c}Number of ligands in the asymmetric unit. [symbol]
indicates data missing or illegible when filed

Structure Determination for the PltM-FAD Intermediate

PltM crystals were soaked in the reservoir solution used to obtained native PltM crystals, with additional 0.5 mM of FAD. The crystals were then gradually transferred to the reservoir solution with 20% v/v PEG 400 and 0.5 mM FAD, prior to quick immersion in liquid nitrogen. The diffraction data were collected and processed as described above. Rigid body refinement followed by restrained refinement were performed starting from the structure of apo PltM. FAD was readily discernable in the omit Fₒ-F_{c} map. Refinement and model building was carried out as described above.

Structure Determination for the Holo PltM-FAD Complex

Wild-type PltM and the L111Y mutant (each at 40 mg/mL) were crystallized by using the reservoir solution composed of 0.1 M Tris pH 8, 0.2 M NaBr, 0.1 M CaCl₂) and 14% PEG 8000 (10% PEG 8000 in case of the PltM L111Y mutant). The crystals were gradually transferred to the cryoprotectant solution (0.1 M Tris pH 8, 0.2 M NaBr, 1 mM FAD, 16% PEG 8000 (14% PEG 8000 for the PltM L111Y mutant), 20% PEG 400 and 1 mM FAD) and incubated overnight. Prior to rapid freezing via liquid nitrogen, crystals were briefly transferred to the cryoprotectant solution containing additionally 0.2 M sodium dithionite. The crystal structures were determined by a procedure analogous to that described above.

Structure Determination for PltM-FAD-Phloroglucinol Complex

Native crystals of PltM were transferred to reservoir solution with 0.5 mM FAD either without or with 1 mM of phloroglucinol for 10 min, then to the cryoprotectant with the same composition, additionally containing 20% v/v PEG 400. After an overnight incubation, the crystals were rapidly frozen in liquid nitrogen. Compounds 1, 2, 3, 8, 21, 23 and 24 were tested. Data collection, processing, and the structure determination were carried out as described above. FAD was clearly discernable in the omit Fₒ-F_{c} electron density map. Out of all substrates tested, only compound 1 (phloroglucinol) yielded omit Fₒ-F_{c} electron density. Phloroglucinol was built into a very strong and featureful polder omit mFₒ-DF_{c} electron density in three out of four substrate binding sites in the asymmetric unit (FIG. 4B).

Data Availability

The crystal structure coordinates and structure factor amplitudes for all crystal structures were deposited in the Protein Data Bank under accession codes 6BZN, 6BZI, 6BZA, 6BZQ, 6BZT and 6BZZ, as described in Tables 5 and 6. NMR spectra, LC-MS, and other chromatographic data are included in the raw format herein.

All publications, patents, and patent applications mentioned in this specification are herein incorporated by reference to the same extent as if each individual publication, patent, or patent application was specifically and individually indicated to be incorporated by reference, including the references set forth in the following list:

### REFERENCES

1. Harris, C. M., Kannan, R., Kopecka, H. & Harris, T. M. The role of the chlorine substituents in the antibiotic vancomycin: preparation and characterization of mono- and didechlorovancomycin. J. Am. Chem. Soc. 107, 6652-6658 (1985).
2. Gerebtzoff, G., Li-Blatter, X., Fischer, H., Frentzel, A. & Seelig, A. Halogenation of drugs enhances membrane binding and permeation. ChemBioChem 5, 676-684 (2004).
3. Ruiz-Castillo, P. & Buchwald, S. L. Applications of palladium-catalyzed C-N cross-coupling reactions. Chem. Rev. 116, 12564-12649 (2016).
4. Tasker, S. Z., Standley, E. A. & Jamison, T. F. Recent advances in homogeneous nickel catalysis. Nature 509, 299-309 (2014).
5. Kuranaga, T., Sesoko, Y. & Inoue, M. Cu-mediated enamide formation in the total synthesis of complex peptide natural products. Nat. Prod. Rep. 31, 514-532 (2014).
6. Latham, J., Brandenburger, E., Shepherd, S. A., Menon, B. R. K. & Micklefield, J. Development of halogenase enzymes for use in synthesis. Chem. Rev. (2017).
7. Poor, C. B., Andorfer, M. C. & Lewis, J. C. Improving the stability and catalyst lifetime of the halogenase RebH by directed evolution. ChemBioChem 15, 1286-1289 (2014).
8. Payne, J. T., Poor, C. B. & Lewis, J. C. Directed evolution of RebH for site-selective halogenation of large biologically active molecules. Angew. Chem. 54, 4226-4230 (2015).
9. Frese, M. & Sewald, N. Enzymatic halogenation of tryptophan on a gram scale. Angew. Chem. 54, 298-301 (2015).
10. Andorfer, M. C. et al. Understanding flavin-dependent halogenase reactivity via substrate activity profiling. ACS Catal. 7, 1897-1904 (2017).
11. Zeng, J. & Zhan, J. A novel fungal flavin-dependent halogenase for natural product biosynthesis. ChemBioChem 11, 2119-2123 (2010).
12. Zeng, J., Lytle, A. K., Gage, D., Johnson, S. J. & Zhan, J. Specific chlorination of isoquinolines by a fungal flavin-dependent halogenase. Bioorg. Med. Chem. Lett. 23, 10011003 (2013).
13. Agarwal, V. et al. Biosynthesis of polybrominated aromatic organic compounds by marine bacteria. Nat. Chem. Biol. 10, 640-647 (2014).
14. Yeh, E., Blasiak, L. C., Koglin, A., Drennan, C. L. & Walsh, C. T. Chlorination by a long-lived intermediate in the mechanism of flavin-dependent halogenases. Biochemistry 46, 1284-1292 (2007).
15. Zhu, X. et al. Structural insights into regioselectivity in the enzymatic chlorination of tryptophan. J. Mol. Biol. 391, 74-85 (2009).
16. Dong, C. et al. Tryptophan 7-halogenase (PrnA) structure suggests a mechanism for regioselective chlorination. Science 309, 2216-2219 (2005).
17. Flecks, S. et al. New insights into the mechanism of enzymatic chlorination of tryptophan. Angew. Chem. 47, 9533-9536 (2008).
18. Podzelinska, K. et al. Chloramphenicol biosynthesis: the structure of CmlS, a flavin-dependent halogenase showing a covalent flavin-aspartate bond. J. Mol. Biol. 397, 316-331 (2010).
19. Menon, B. R. et al. Structure and biocatalytic scope of thermophilic flavin-dependent halogenase and flavin reductase enzymes Org. Biomol. Chem. 14, 9354-9361 (2016).
20. Bitto, E. et al. The structure of flavin-dependent tryptophan 7-halogenase RebH. Proteins 70, 289-293 (2008).
21. Nowak-Thompson, B., Chaney, N., Wing, J. S., Gould, S. J. & Loper, J. E. Characterization of the pyoluteorin biosynthetic gene cluster of Pseudomonas fluorescens Pf-5. J Bacteriol. 181, 2166-2174 (1999).
22. Yan, Q., Philmus, B., Chang, J. H. & Loper, J. E. Novel mechanism of metabolic co-regulation coordinates the biosynthesis of secondary metabolites in Pseudomonas protegens. Elife 6, e22835 (2017).
23. Dorrestein, P. C., Yeh, E., Garneau-Tsodikova, S., Kelleher, N. L. & Walsh, C. T. Dichlorination of a pyrrolyl-S-carrier protein by FADH2-dependent halogenase PltA during pyoluteorin biosynthesis. Proc. Natl. Acad. Sci., U.S.A 102, 13843-13848 (2005).
24. Pang, A. H., Garneau-Tsodikova, S. & Tsodikov, O. V. Crystal structure of halogenase PltA from the pyoluteorin biosynthetic pathway. J. Struct. Biol. 192, 349-357 (2015).
25. Liebschner, D. et al. Polder maps: improving OMIT maps by excluding bulk solvent. Acta Crystallogr. D Struct. Biol. 73, 148-157 (2017).
26. Yeh, E. et al. Flavin redox chemistry precedes substrate chlorination during the reaction of the flavin-dependent halogenase RebH. Biochemistry 45, 7904-7912 (2006).
27. Ortiz-Maldonado, M., Ballou, D. P. & Massey, V. A rate-limiting conformational change of the flavin in p-hydroxybenzoate hydroxylase is necessary for ligand exchange and catalysis: studies with 8-mercapto- and 8-hydroxy-flavins. Biochemistry 40, 1091-1101 (2001).
28. Payne, J. T., Andorfer, M. C. & Lewis, J. C. Regioselective arene halogenation using the FAD-dependent halogenase RebH. Angew. Chem. 52, 5271-5274 (2013).
29. Kuzmic, P. Program DYNAFIT for the analysis of enzyme kinetic data: application to HIV proteinase. Anal. Biochem. 237, 260-273 (1996).
30. Tan, H. et al. Structure-activity relationships and optimization of acyclic acylphloroglucinol analogues as novel antimicrobial agents. Eur. J. Med. Chem. 125, 492-499 (2017).
31. Ho, S. N., Hunt, H. D., Horton, R. M., Pullen, J. K. & Pease, L. R. Site-directed mutagenesis by overlap extension using the polymerase chain reaction. Gene 77, 51-59 (1989).
32. Pongtharangkul, T. et al. Kinetic properties and stability of glucose dehydrogenase from Bacillus amyloliquefaciens SB5 and its potential for cofactor regeneration. AMB Express 5, 68 (2015).
33. Otwinowski, Z. & Minor, W. Processing of X-ray diffraction data collected in oscillation mode. Methods Enzymol. 276, 307-326 (1997).
34. Sheldrick, G. M. Experimental phasing with SHELXC/D/E: combining chain tracing with density modification. Acta Crystallogr. D 66, 479-485 (2010).
35. Adams, P. D. et al. PHENIX: a comprehensive Python-based system for macromolecular structure solution. Acta Crystallogr. D 66, 213-221 (2010).
36. Emsley, P. & Cowtan, K. Coot: model-building tools for molecular graphics. Acta Crystallogr. D 60, 2126-2132 (2004).
37. Murshudov, G. N. et al. REFMAC5 for the refinement of macromolecular crystal structures. Acta Crystallogr. D 67, 355-367 (2011).
38. McCoy, A. J. et al. Phaser crystallographic software. J. Appl. Crystallogr 40, 658-674 (2007).
39. Winn, M. D. et al. Overview of the CCP4 suite and current developments. Acta Crystallogr D 67, 235-242 (2011).
40. Zhu, X.; De Laurentis, W.; Leang, K.; Herrmann, J.; Ihlefeld, K.; van Pee, K. H.; Naismith, J. H., Structural insights into regioselectivity in the enzymatic chlorination of tryptophan. J. Mol. Biol. 2009, 391 (1), 74-85.
41. Ortega, M. A.; Cogan, D. P.; Mukherjee, S.; Garg, N.; Li, B.; Thibodeaux, G. N.; Maffioli, S. I.; Donadio, S.; Sosio, M.; Escano, J.; Smith, L.; Nair, S. K.; van der Donk, W. A., Two flavoenzymes catalyze the post-translational generation of 5-chlorotryptophan and 2-aminovinyl-cysteine during NAI-107 biosynthesis. ACS Chem. Biol. 2017, 12 (2), 548-557.
42. Shepherd, S. A.; Menon, B. R.; Fisk, H.; Struck, A. W.; Levy, C.; Leys, D.; Micklefield, J., A structure-guided switch in the regioselectivity of a tryptophan halogenase. ChemBioChem 2016, 17 (9), 821-824.
43. Dong, C.; Flecks, S.; Unversucht, S.; Haupt, C.; van Pee, K. H.; Naismith, J. H., Tryptophan 7-halogenase (PrnA) structure suggests a mechanism for regioselective chlorination. Science 2005, 309 (5744), 2216-2219.
44. Flecks, S.; Patallo, E. P.; Zhu, X.; Ernyei, A. J.; Seifert, G.; Schneider, A.; Dong, C.; Naismith, J. H.; van Pee, K. H., New insights into the mechanism of enzymatic chlorination of tryptophan. Angew. Chem. 2008, 47 (49), 9533-9536.
45. Shepherd, S. A.; Karthikeyan, C.; Latham, J.; Struck, A. W.; Thompson, M. L.; Menon, B. R.; Styles, M. Q.; Levy, C.; Leys, D.; Micklefield, J., Extending the biocatalytic scope of regiocomplementary flavin-dependent halogenase enzymes. Chem. Sci. 2015, 6, 3454-3460.
46. Bitto, E.; Huang, Y.; Bingman, C. A.; Singh, S.; Thorson, J. S.; Phillips, G. N., Jr., The structure of flavin-dependent tryptophan 7-halogenase RebH. Proteins 2008, 70 (1), 289-293.
47. Yeh, E.; Blasiak, L. C.; Koglin, A.; Drennan, C. L.; Walsh, C. T., Chlorination by a long-lived intermediate in the mechanism of flavin-dependent halogenases. Biochemistry 2007, 46 (5), 1284-1292.
48. Menon, B. R.; Latham, J.; Dunstan, M. S.; Brandenburger, E.; Klemstein, U.; Leys, D.; Karthikeyan, C.; Greaney, M. F.; Shepherd, S. A.; Micklefield, J., Structure and biocatalytic scope of thermophilic flavin-dependent halogenase and flavin reductase enzymes. Org. Biomol. Chem. 2016, 14 (39), 9354-9361.
49. Fraley, A. E.; Garcia-Borras, M.; Tripathi, A.; Khare, D.; Mercado-Marin, E. V.; Tran, H.; Dan, Q.; Webb, G. P.; Watts, K. R.; Crews, P.; Sarpong, R.; Williams, R. M.; Smith, J. L.; Houk, K. N.; Sherman, D. H., Function and structure of MalA/MalA′, iterative halogenases for late-stage C-H functionalization of indole alkaloids. J. Am. Chem. Soc. 2017, 139 (34), 12060-12068.
50. Pang, A. H.; Garneau-Tsodikova, S.; Tsodikov, O. V., Crystal structure of halogenase PltA from the pyoluteorin biosynthetic pathway. J. Struct. Biol. 2015, 192 (3), 349-357.
51. El Gamal, A.; Agarwal, V.; Diethelm, S.; Rahman, I.; Schorn, M. A.; Sneed, J. M.; Louie, G. V.; Whalen, K. E.; Mincer, T. J.; Noel, J. P.; Paul, V. J.; Moore, B. S., Biosynthesis of coral settlement cue tetrabromopyrrole in marine bacteria by a uniquely adapted brominasethioesterase enzyme pair. Proc. Natl. Acad. Sci., U.S.A 2016, 113 (14), 3797-3802.
52. Buedenbender, S.; Rachid, S.; Muller, R.; Schulz, G. E., Structure and action of the myxobacterial chondrochloren halogenase CndH: a new variant of FAD-dependent halogenases. J. Mol. Biol. 2009, 385 (2), 520-530.
53. Podzelinska, K.; Latimer, R.; Bhattacharya, A.; Vining, L. C.; Zechel, D. L.; Jia, Z., Chloramphenicol biosynthesis: the structure of CmlS, a flavin-dependent halogenase showing a covalent flavin-aspartate bond. J. Mol. Biol. 2010, 397 (1), 316-331.
54. Lovell, S. C.; Davis, I. W.; Arendall, W. B., 3rd; de Bakker, P. I.; Word, J. M.; Prisant, M. G.; Richardson, J. S.; Richardson, D. C., Structure validation by Calpha geometry: phi,psi and Cbeta deviation. Proteins 2003, 50 (3), 437-450.
55. Krissinel, E.; Henrick, K., Secondary-structure matching (SSM), a new tool for fast protein structure alignment in three dimensions. Acta Crystallogr. D Biol Crystallogr. 2004, 60 (Pt 12 Pt 1), 2256-2268.
56. Robert, X.; Gouet, P., Deciphering key features in protein structures with the new ENDscript server. Nucl. Acids Res. 2014, 42 (Web Server issue), W320-324.
57. Kuzmic, P., Program DYNAFIT for the analysis of enzyme kinetic data: application to HIV proteinase. Anal. Biochem. 1996, 237 (2), 260-273.

While the disclosure is susceptible to various modifications and alternative forms, specific embodiments thereof have been shown by way of example in the drawings and are herein described below in detail. It should be understood, however, that the description of specific embodiments is not intended to limit the disclosure to cover all modifications, equivalents and alternatives falling within the spirit and scope of the disclosure as defined by the appended claims.

What is claimed is:

1. A halogenation system comprising:
PltM; and
a solid support;
wherein the PltM is immobilized on the solid support.

2. The system of claim 1, wherein the solid support is a resin.

3. The system of claim 2, wherein the resin is an agarose resin.

4. The system of claim 2, wherein the resin is packed into a spin column.

5. The system of claim 1, further comprising one or more enzymes immobilized on the solid support.

6. The system of claim 5, wherein the one or more enzymes include a flavin adenine dinucleotide (FAD) reductase.

7. The system of claim 6, wherein the FAD reductase includes SsuE.

8. The system of claim 5, wherein the one or more enzymes include a NADPH regenerator.

9. The system of claim 8, wherein the NADPH regenerator includes glucose dehydrogenase (GDH).

10. The system of claim 5, wherein the one or more enzymes include a flavin adenine dinucleotide (FAD) reductase and a NADPH regenerator; and wherein the FAD reductase and the NADPH regenerator are immobilized on the solid support.

11. The system of claim 10, wherein the FAD reductase is SsuE.

12. The system of claim 11, wherein the NADPH regenerator is glucose dehydrogenase (GDH).

13. The system of claim 12, wherein the PltM, SsuE, and GDH are packed into a spin column.

14. A method of halogenating a substrate, the method comprising running a substrate and reaction solution through the halogenation system of claim 1.

15. The method of claim 14, wherein halogenation system further comprises SsuE and glucose dehydrogenase (GDH).

16. The method of claim 14, wherein the substrate is a phenyl compound with one or more electron donating groups.

17. The method of claim 16, wherein the phenyl compound is selected from the group consisting of phenolic derivatives, aniline derivatives, short-acting b2 adrenoreceptor agonists, natural products, and a combination thereof.

18. The method of claim 14, wherein the substrate is mono-halogenated.

19. The method of claim 14, wherein the substrate is di-halogenated.

20. A halogenated compound selected from the group consisting of 4,6-diCl-3, 4,6-diCl-8, 2,4-diCl-9, 2,6-diCl-11, 3,5-diCl-15, 4,6-diCl-16, 4,6-diCl-18, 4-Cl-23, and 4,6-diBr-3.


SEQUENCE LISTING (13 sequences)

SEQ ID NO: 1 - DNA, 32 bp, Artificial
  PltM Mutant Primer
cgcctgcgggatcgcgctgggcttcagttttg

SEQ ID NO: 2 - DNA, 35 bp, Artificial
  PltM Mutant Primer
catactcgagctagactttgaggatgaaacgattg

SEQ ID NO: 3 - DNA, 32 bp, Artificial
  PltM Mutant Primer
caaaactgaagcccagcgcgatcccgcaggcg

SEQ ID NO: 4 - DNA, 35 bp, Artificial
  PltM Mutant Primer
gcagctctcatatgaatcagtacgacgtcattatc

SEQ ID NO: 5 - DNA, 33 bp, Artificial
  PltM Mutant Primer
cttgtggccccgccgtataaggtgccggaagcc

SEQ ID NO: 6 - DNA, 33 bp, Artificial
  PltM Mutant Primer
ggcttccggcaccttatacggcggggccacaag

SEQ ID NO: 7 - DNA, 30 bp, Artificial
  PltM Mutant Primer
ctggctcagcggctataacctgggcagtgc

SEQ ID NO: 8 - DNA, 30 bp, Artificial
  PltM Mutant Primer
gcactgcccaggttatagccgctgagccag

SEQ ID NO: 9 - DNA, 34 bp, Artificial
  Glucose Dehydrogenase Forward Primer
aggatgcatatgtatccggatttaaaaggaaaag

SEQ ID NO: 10 - DNA, 34 bp, Artificial
  Glucose Dehydrogenase Reverse Primer
cgctttctcgagttaaccgcggcctgcctggaat

SEQ ID NO: 11 - protein, 502 aa, Pseudomonas fluorescens
MNQYDVIIIGSGIAGALTGAVLAKSGLNVLILDSAQHPRFSVGEAATPESGFLLRLLSKRFDIPEIAYLSHPDKIIQHVGSSACGIKLGFSFAWHQENAPSSPDHLVAPPLKVPEAHLFRQDIDYFALMIALKHGAESRQNIKIESISLNDDGVEVALSNAAPVKAAFIIDAAAQGSPLSRQLGLRTTEGLATDTCSFFTHMLNVKSYEDALAPLSRTRSPIELFKSTLHHIFEEGWLWVIPFNNHPQGTNQLCSIGFQFNNAKYRPTEAPEIEFRKLLKKYPAIGEHFKDAVNAREWIYAPRINYRSVQNVGDRFCLLPQATGFIDPLFSRGLITTFESILRLAPKVLDAARSNRWQREQFIEVERHCLNAVATNDQLVSCSYEAFSDFHLWNVWHRVWLSGSNLGSAFLQKLLHDLEHSGDARQFDAALEAVRFPGCLSLDSPAYESLFRQSCQVMQQAREQARPVAETANALHELIKEHEAELLPLGYSRISNRFILKV

SEQ ID NO: 12 - protein, 449 aa, Pseudomonas fluorescens
MSDHDYDVVIIGGGPAGSTMASYLAKAGVKCAVFEKELFEREHVGESLVPATTPVLLEIGVMEKIEKANFPKKFGAAWTSADSGPEDKMGFQGLDHDFRSAEILFNERKQEGVDRDFTFHVDRGKFDRILLEHAGSLGAKVFQGVEIADVEFLSPGNVIVNAKLGKRSVEIKAKMVVDASGRNVLLGRRLGLREKDPVFNQFAIHSWFDNFDRKSATQSPDKVDYIFIHFLPMTNTWVWQIPITETITSVGVVTQKQNYTNSDLTYEEFFWEAVKTRENLHDALKASEQVRPFKKEADYSYGMKEVCGDSFVLIGDAARFVDPIFSSGVSVALNSARIASGDIIEAVKNNDFSKSSFTHYEGMIRNGIKNWYEFITLYYRLNILFTAFVQDPRYRLDILQLLQGDVYSGKRLEVLDKMREIIAAVESDPEHLWHKYLGDMQVPTAKPAF

SEQ ID NO: 13 - protein, 530 aa, Lentzea aerocolonigenes
MSGKIDKILIVGGGTAGWMAASYLGKALQGTADITLLQAPDIPTLGVGEATIPNLQTAFFDFLGIPEDEWMRECNASYKVAIKFINWRTAGEGTSEARELDGGPDHFYHSFGLLKYHEQIPLSHYWFDRSYRGKTVEPFDYACYKEPVILDANRSPRRLDGSKVTNYAWHFDAHLVADFLRRFATEKLGVRHVEDRVEHVQRDANGNIESVRTATGRVFDADLFVDCSGFRGLLINKAMEEPFLDMSDHLLNDSAVATQVPHDDDANGVEPFTSAIAMKSGWTWKIPMLGRFGTGYVYSSRFATEDEAVREFCEMWHLDPETQPLNRIRFRVGRNRRAWVGNCVSIGTSSCFVEPLESTGIYFVYAALYQLVKHFPDKSLNPVLTARFNREIETMFDDTRDFIQAHFYFSPRTDTPFWRANKELRLADGMQEKIDMYRAGMAINAPASDDAQLYYGNFEEEFRNFWNNSNYYCVLAGLGLVPDAPSPRLAHMPQATESVDEVFGAVKDRQRNLLETLPSLHEFLRQQHGR